(19) United States
(12) Patent Application Publication
(10) Pub. No.: US 2022/0049251 A1
(43) Pub. Date: Feb. 17, 2022

(54) dsRNA Directed to Coronavirus Proteins

(71) Applicant: Nitto Denko Corporation, Osaka (JP)

(72) Inventors: Jens Harborth, San Diego, CA (US); Cima Cina, San Diego, CA (US); Bharat Majeti, San Diego, CA (US); Roger Adami, Carlsbad, CA (US); Wenbin Ying, San Diego, CA (US)

(21) Appl. No.: 17/303,051

(22) Filed: May 19, 2021

Related U.S. Application Data
(60) Provisional application No. 63/027,901, filed on May 20, 2020.

(51) Int. Cl. C12N 15/113; C12N 15/86

(52) U.S. Cl. CPC C12N 15/113; C12N 15/86; C12N 2310/11; C12N 2310/321; C12N 2310/313

(57) ABSTRACT

Disclosed are compositions that include double-stranded ribonucleic acid (dsRNA) constructs that inhibit expression or translation of a Coronavirus protein, and methods of using them to treat MERS, SARS, Covid-19 and/or symptoms thereof. Some embodiments relate to a method for preventing, treating, inhibiting, or ameliorating a MERS, SARS, or Covid-19 symptom using a composition herein disclosed. In some embodiments the composition targets the lung.

### INCORPORATION BY REFERENCE TO PRIORITY APPLICATION

This application claims priority to U.S. Provisional Application No. 63/027,901, filed May 20, 2020, which is hereby incorporated by reference in its entirety.

### REFERENCE TO SEQUENCE LISTING

The present application is being filed along with a Sequence Listing in electronic format. The Sequence Listing is provided as a file entitled HRAK001016AUS_SQLIST, created Oct. 18, 2021, which is approximately 87 Kb in size. The information in the electronic format of the Sequence Listing is incorporated herein by reference in its entirety.

### FIELD

Described herein are nucleic acids such as double-stranded ribonucleic acid (dsRNA) constructs that inhibit expression or translation of a Coronavirus protein, and methods of using them to treat Middle East Respiratory Syndrome (MERS), Severe Acute Respiratory Syndrome (SARS), Covid-19 and symptoms thereof.

### BACKGROUND

There is an urgent need for compositions and methods for modulating the expression of Coronavirus proteins, and particularly the proteins associated with the diseases or respiratory conditions known as MERS, SARS and Covid-19. Although many infected people have mild symptoms, in other cases they suffer severe breathing problems and death, particularly among vulnerable populations. Although several vaccines have been developed, there is currently no cure for MERS, SARS or Covid-19. The need to identify effective therapies is paramount as the lives and livelihoods of millions of people worldwide are threatened.

### SUMMARY

Aspects of the present disclosure relate to biopharmaceuticals and therapeutics composed of nucleic acid-based molecules. More particularly, some embodiments concern compounds and compositions comprising RNA interference (e.g., siRNA) molecules for modulating the expression of Coronavirus proteins and methods of therapy, which utilize these molecules. Some embodiments, concern, for example, siRNA molecules, which are configured to or capable of gene silencing of Coronaviruses and by this gene silencing are useful for methods of preventing, treating, inhibiting or ameliorating coronavirus diseases and symptoms thereof, such as MERS, SARS and Covid-19.

Some embodiments relate to double-stranded ribonucleic acid (dsRNA) molecules, which are configured to or capable of inhibiting expression of Coronavirus proteins. For example, various embodiments relate to a dsRNA for inhibiting expression of a MERS-CoV protein, a SARS-CoV protein and/or a SARS-CoV-2 protein. In some embodiments, the dsRNA includes a sense strand and an antisense strand comprising a region of complementarity complementary to an mRNA encoding a Coronavirus protein, wherein each strand is at least 15 nucleotides in length.

In some embodiments, the strands form a duplex region, and wherein the antisense strand is at least 15 contiguous nucleotides from any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, or 60. Some embodiments include one or more single-stranded overhang(s) of one or more nucleotides. In some embodiments, at least one end of the dsRNA has a single-stranded nucleotide overhang of 1 to 4, or 1, 2, 3, 4 or 5 nucleotides. In some embodiments, the antisense strand of the dsRNA has a 1-5 nucleotide overhang at each of the 3′ end and the 5′ end. In some embodiments, each strand comprises a 3′ overhang consisting of 2 nucleotides. In some embodiments, each strand comprises a 3′ overhang consisting of UU. Some embodiments include a nucleotide modification that causes the dsRNA to have increased stability in a biological sample. Some embodiments include at least one modified nucleotide, wherein said modified nucleotide is selected from the group of: a 2′-O-methyl modified nucleotide, a nucleotide comprising a 5′-phosphorothioate group, a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural base comprising nucleotide. In some embodiments, each strand comprises a 3′ overhang consisting of mUmU, wherein “m” refers to a 2′-OMe-substitution in the next nucleotide.

In some embodiments, the dsRNA molecules comprise a sense strand; and an antisense strand; wherein the strands form a duplex region and, wherein the sense strand or antisense strand comprises at least 15 contiguous nucleotides from any one of SEQ ID NOs: 1-168.

In one aspect, each strand of the dsRNA is no more than 30 nucleotides in length. At least one strand can include a 3′ overhang of at least 1 nucleotide, e.g., 2 nucleotides.

In some embodiments, the dsRNA includes one or more modified nucleotides. For example, the dsRNA can include a nucleotide modification that causes the dsRNA to have increased stability in a biological sample. In one embodiment, the dsRNA includes at least one modified nucleotide, e.g., a 2′-O-methyl modified nucleotide, a nucleotide comprising a 5′-phosphorothioate group, or a terminal nucleotide linked to a cholesteryl derivative or dodecanoic acid bisdecylamide group. In other embodiments the modified nucleotide is a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide. The dsRNA of the disclosure can include at least one 2′-O-methyl modified nucleotide and at least one 2′-deoxythymidine-3′-phosphate nucleotide comprising a 5′-phosphorothioate group.

In some of these embodiments, the overhangs include a modified nucleotide. In some of these embodiments, 1, 2, 3, 4 or 5, or a number of residues that is within a range defined by any of the two aforementioned numbers, of the last 5 nucleotides on the 3′ or 5′ end or both of the antisense strand of the dsRNA inhibitors of Coronavirus proteins (e.g., wherein the sense strand or antisense strand comprises at least 15 contiguous nucleotides from any one of SEQ ID NOs: 1-168 include a modified nucleotide. Some of these embodiments include a 2′-deoxy-2′-fluoro substituted nucleotide in the duplex region.

In some embodiments of the contemplated dsRNA Coronavirus inhibitors, the dsRNA inhibits expression of Coronavirus mRNA in lung and/or nasal cells with an EC50 of less than 1000, 500, 250, 100, 75, 50, or 25 pM, or a pM range encompassing any two of the aforementioned concentrations. In some embodiments of the contemplated dsRNA Coronavirus inhibitors, the dsRNA Coronavirus inhibitor decreases or prevents expression of one or more Coronavirus proteins in vivo, such as one or more targets listed in Tables 1-5.

Additional embodiments relate to pharmaceutical compositions comprising one or more of the contemplated dsRNA Coronavirus inhibitors described herein and a pharmaceutically acceptable carrier. In some embodiments, the carrier comprises a lipid or a liposome. Some embodiments also concern a vector comprising one or more of the contemplated dsRNA Coronavirus inhibitors described herein. Some embodiments also relate to a cell comprising one or more of the contemplated dsRNA Coronavirus inhibitors or vectors described herein.

More embodiments relate to methods for inhibiting, ameliorating, preventing, or treating Coronavirus protein-mediated diseases or symptoms associated therewith such as MERS, SARS and/or Covid-19, wherein the methods comprise administering to a subject in need, such as a human, one or more of the contemplated dsRNA Coronavirus inhibitors, pharmaceutical compositions, or vectors, described herein and, optionally identifying said subject as one being amenable to such therapies (e.g., by clinical or diagnostic evaluation) and, optionally measuring the inhibition of Coronavirus or inhibition, amelioration, or treatment of the Coronavirus protein-mediated disease or symptom associated therewith (e.g., by clinical or diagnostic evaluation).

Accordingly, some embodiments relate to a method for preventing, treating, inhibiting, or ameliorating one or more symptoms of MERS, SARS and/or Covid-19 in a mammal in need thereof, such as a human. In some embodiments, the method includes administering to the mammal a therapeutically effective amount of a composition comprising a specific inhibitor or antagonist of a Coronavirus protein, such as any one or more of the contemplated dsRNA Coronavirus inhibitors described herein (e.g., a dsRNA Coronavirus inhibitor described herein, wherein the sense strand or antisense strand comprises at least 15 contiguous nucleotides from any one of SEQ ID NOs: 1-168.

In more embodiments, the specific inhibitor or antagonist of a Coronavirus protein comprises a Coronavirus protein expression inhibitor. In some embodiments, the Coronavirus protein expression inhibitor comprises a Coronavirus siRNA. In some embodiments, the Coronavirus siRNA comprises a dsRNA, as described herein. In some embodiments, the Coronavirus siRNA comprises a nanoparticle. In some embodiments, the symptoms of MERS, SARS and/or Covid-19 (which are prevented, treated, ameliorated, or inhibited by providing one or more of the therapies or compositions described herein) include a positive test for presence of a Coronavirus (e.g., in a sample obtained by nasal pharyngeal swab) and/or one or more clinical symptoms such as cough, shortness of breath or difficulty breathing, fever, chills, muscle pain, sore throat, and/or new loss of taste or smell; and, after receiving such therapy, these symptoms are reduced as compared to a mammal, such as a human, which has MERS, SARS and/or Covid-19, or are reduced to levels comparable to a mammal, such as a human, which does not have MERS, SARS and/or Covid-19. In some embodiments, the one or more symptoms of MERS, SARS and/or Covid-19 are prevented, treated, inhibited or ameliorated within 1 days, 3 days, 5 days, or 7 days, or within a time period existing within a range defined by any two of the aforementioned numbers of days, upon administration of the specific inhibitor or antagonist of a Coronavirus, as described herein. In some embodiments, one or more symptoms of MERS, SARS and/or Covid-19 are ameliorated or inhibited or reduced by at least 25%, 50%, 75%, or 100%, or by an amount that is within a range defined by any two of the aforementioned percentages, upon administration of the specific inhibitor or antagonist of a Coronavirus, as described herein.

Some embodiments also relate to a pharmaceutical composition comprising nanoparticles encapsulating an siRNA comprising a specific inhibitor or antagonist of Coronavirus, as set forth herein. For instance, any one or more of the contemplated dsRNA Coronavirus inhibitors described herein (e.g., a dsRNA Coronavirus inhibitor described herein, wherein the sense strand or antisense strand comprises at least 15 contiguous nucleotides from any one of SEQ ID NOs: 1-168 can be provided in a nanoparticle encapsulating said dsRNA).

Accordingly, several embodiments described herein relate to dsRNA molecules suitable for or configured to inhibit expression of Coronavirus and thereby prevent, inhibit, ameliorate, or treat a Coronavirus protein-mediated disease or condition or symptom associated therewith, such as MERS, SARS and/or Covid-19. In some embodiments, the dsRNA includes a sense strand and an antisense strand, the antisense strand comprising a region of complementarity, which comprises at least 15 contiguous nucleotides from the nucleotide sequence of any one of the even-numbered SEQ ID NOs: in the range of 2 to 168 (e.g., SEQ ID NOs: 2, 4, 6, . . . 164, 166, 168). Said dsRNAs can be incorporated into pharmaceutical compositions, such as compositions comprising a liposome, nanoparticle, and can be administered to subjects, such as humans, which have a Coronavirus protein-mediated disease or symptom or condition thereof so as to treat, inhibit, ameliorate, or reduce said disease, symptom, or condition, such as MERS, SARS and/or Covid-19.

More embodiments of the Coronavirus inhibitors contemplated herein relate to sense or antisense polynucleotide agents for inhibiting expression of Coronavirus. In some embodiments, the agent includes 4 to 50, or about 4 to about 50, contiguous nucleotides, wherein at least one of the contiguous nucleotides is a modified nucleotide, and wherein the nucleotide sequence of the agent has at least 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity or has a sequence identity that is within a range defined by any two of the aforementioned percentages, over its entire length to the equivalent region of the nucleotide sequence of any one of SEQ ID NOs: 1-168 or the complement thereof.

Additional embodiments relate to a nucleic acid molecule for inhibiting expression of a Coronavirus, wherein the nucleic acid molecule includes a sense strand and an antisense strand; wherein the strands form a duplex region; and wherein the sense strand and the antisense strand each have a sequence in accordance with any one of SEQ ID NOs: 61-120, wherein nucleotides A, G, C and U refer to ribo-A (adenosine), ribo-G (guanosine), ribo-C (cytidine) and ribo-U (uridine), respectively. Additional embodiments relate to a nucleic acid molecule for inhibiting expression of a Coronavirus, wherein the nucleic acid molecule includes a sense strand and an antisense strand; wherein the strands form a duplex region; and wherein the sense strand and the antisense strand each have a sequence in accordance with any one of SEQ ID NOs: 121-168.

More embodiments relate to nucleic acid molecules for inhibiting expression of a Coronavirus, wherein the nucleic acid molecules include a sense strand and an antisense strand; wherein the strands form a duplex region; and wherein the sense strand and the antisense strand each have a sequence in accordance with any one of SEQ ID NOs: 121-168 wherein the nucleotide upper case letters A, G, C and U refer to ribo-A (adenosine), ribo-G (guanosine), ribo-C (cytidine) and ribo-U (uridine), respectively; wherein the lower case letters a, g, c and u refer to 2-deoxy-A, 2-deoxy-G, 2-deoxy-C and 2-deoxy-U, respectively; and wherein mA, mG, mC and mU refer to 2′OMe-modified A, 2′OMe-modified G, 2′OMe-modified C and 2′OMe-modified U, respectively.

The following provides greater detail on some of the preferred alternatives.

An embodiment provides a double-stranded ribonucleic acid (dsRNA) for inhibiting expression of a Coronavirus protein, comprising a sense strand and an antisense strand comprising a region of complementarity complementary to an mRNA encoding the protein, wherein the strands form a duplex region, and wherein the antisense strand is at least 15 contiguous nucleotides from any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, or has at least 90%, 91%, 92%, 93%, 94%, 95%, 96%, 97%, 98%, or 99% sequence identity to any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, or a sequence identity to any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, which is within a range defined by any two of the aforementioned percentages. In various embodiments, the Coronavirus protein is a MERS-CoV protein, a SARS-CoV protein or a SARS-CoV-2 protein.

Another embodiment provides a pharmaceutical composition comprising a dsRNA as described herein and a pharmaceutically acceptable carrier. In various embodiments, the pharmaceutical composition comprises:
(a) an effective amount of a dsRNA as described herein;
(b) a compound having the following Formula II:

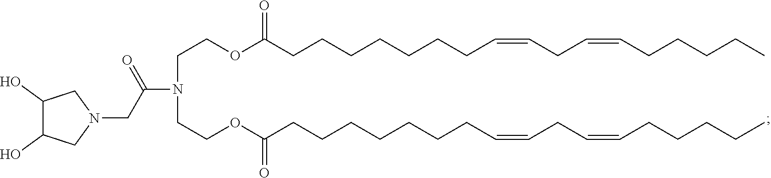 [SMILES: CCCCC/C=C\C/C=C\CCCCCCCC(=O)OCCN(CCOC(=O)CCCCCCC/C=C\C/C=C\CCCCC)C(=O)CN1CC(O)C(O)C1]

(c) a DSPE lipid comprising a polyethyleneglycol (PEG) region, a multi-branched PEG region, a methoxypolyethyleneglycol (mPEG) region, a carbonyl-methoxypolyethyleneglycol region, or a polyglycerine region;
(d) a sterol lipid; and
(e) one or more neutral lipids.

Another embodiment provides a method for inhibiting, preventing, or treating a Coronavirus infection, the method comprising administering to a subject in need thereof a therapeutically effective amount of a dsRNA as described herein. In various embodiments, the dsRNA is administered in the form of a pharmaceutical composition or a pharmaceutical solution. In various embodiments, the subject is need has or is at risk of having MERS, SARS or Covid-19.

These and other embodiments are described in greater detail below.

### BRIEF DESCRIPTION OF THE DRAWINGS

FIG. 1A shows enhanced distribution to lung in vivo mouse using an embodiment of a pharmaceutical formulation that provides delivery of an API to the lung as described in International Application No. PCT/US2019/061702 and U.S. Patent Publication No. 2020/0157540. The lipid nanoparticle (LNP) pharmaceutical formulation contained an API (siRNA targeted to Coronavirus, (SEQ ID NO: 61/62 (based on SEQ ID NO: 1/2) or SEQ ID NO: 71/72 (based on SEQ ID NO: 11/12)), an ionizable lipid Compound A 25 mol %, cholesterol 30 mol %, DOPE 20 mol %, DOPC 20 mol %, and DSPE-mPEG-2000 5 mol %. Organs were harvested 4 hours after injection of a dose at 4 mg/kg in naïve animals, with 5 animals per group. The accumulation of siRNA in the organ was measured by fluorescence.

FIG. 1B shows the ratio of distribution of lung to liver in vivo mouse for embodiments of formulations of this invention. The administered LNP composition of the formulation contains API (siRNA targeted to Coronavirus, (SEQ ID NOS: 61/62 or 71/72)), Compound A 25 mol %, cholesterol 30 mol %, DOPE 20 mol %, DOPC 20 mol %, and DSPE-mPEG-2000 5 mol %. The results showed that the ratio of the distribution of active agent to lung over liver in vivo mouse was 2-fold.

FIG. 2 shows a bar graph of R/L ratio as a function of concentration for various dsRNA having the indicated sequence numbers. The data shows that expression is reduced at higher dosages, indicating a dose/response effect.

FIG. 3 shows a bar graph of R/L ratio as a function of concentration for various dsRNA having the indicated sequence numbers. The data shows that expression is reduced at higher dosages, indicating a dose/response effect.

FIG. 4 shows a bar graph of R/L ratio as a function of concentration for various dsRNA having the indicated sequence numbers. The data shows that expression is reduced at higher dosages, indicating a dose/response effect.

FIG. 5 shows a bar graph of R/L ratio as a function of concentration for various dsRNA having the indicated sequence numbers. The data shows that expression is reduced at higher dosages, indicating a dose/response effect.

FIG. 6 shows a bar graph of R/L ratio as a function of concentration for various dsRNA having the indicated sequence numbers. The data shows that expression is reduced at higher dosages, indicating a dose/response effect.

FIG. 7 shows a bar graph of R/L ratio as a function of concentration for various dsRNA having the indicated sequence numbers. The data shows that expression is reduced at higher dosages, indicating a dose/response effect.

FIG. 8 shows a bar graph of R/L ratio as a function of concentration for various dsRNA having the indicated sequence numbers. The data shows that expression is reduced at higher dosages, indicating a dose/response effect.

FIG. 9 shows a bar graph of R/L ratio as a function of concentration for various dsRNA having the indicated sequence numbers. The data shows that expression is reduced at higher dosages, indicating a dose/response effect.

FIG. 10 shows a bar graph of R/L ratio as a function of concentration for various dsRNA having the indicated sequence numbers. The data shows that expression is reduced at higher dosages, indicating a dose/response effect.

FIG. 11 shows a bar graph of R/L ratio as a function of concentration for various dsRNA having the indicated sequence numbers. The data shows that expression is reduced at higher dosages, indicating a dose/response effect.

FIG. 12 shows a nucleic acid sequence (SEQ ID. NO. 169) of the complete genome of an example of SARS-CoV-2.

FIG. 13 shows a sequence (SEQ. ID. NO. 170) of the multiple cloning site insert used in the Example Protocol below.

FIG. 14 shows a bar graph of pfu/mL ratio from a plaque assay as a function of either 5 nM or 25 nM treatment of siRNA (SEQ ID NOS: 61/62, 71/72, 141/142 or 157/158) in VeroE6 cells contacted with coronavirus.

FIG. 15 shows the relationship between pfu/mL ratio from a plaque assay in VeroE6 cells contacted with coronavirus, and the increasing concentration of siRNA (SEQ ID NO: 141/142).

FIG. 16 shows an example set of plates from a plaque assay, wherein the first panel depicts the dilution of coronavirus administered, the second panel depicts plaques in VeroE6 cells treated with 0.008 nM siRNA (SEQ ID NO: 141/142), and the third panel depicts plaques in VeroE6 cells treated with 25 nM siRNA (SEQ ID NO: 141/142).

FIG. 17 shows the estimated IC50 values of siRNA (SEQ ID NO: 141/142) in reducing coronavirus-induced plaques, as quantified using GraphPad Prism. Error bars represent standard deviations.

FIG. 18 shows a bar graph of percent SARS-CoV-2 Spike/RPL0 expression as a function of treatment with either 25 nM or 5 nM siRNA (SEQ ID NOS: 61/62, 71/72, 141/142 or 157/158). Total RNA was isolated and real-time PCR analysis performed using probes against the spike protein gene.

FIG. 19 shows the estimated IC50 values of siRNA (SEQ ID NOS: 141/142 and 157/158) in reducing SARS-CoV-2 spike protein expression, as quantified using GraphPad Prism. Vero-E6 cells were transfected with different concentrations of the siRNA (5-fold serial dilution) for 24 hours and then infected with SARS-CoV-2 isolate USA-WA1/2020 at a MOI of 0.5 for 24 hours. Total RNA was isolated and real-time PCR analysis performed using probes against the spike protein gene. Results represent mean±SD (n=3).

FIG. 20 shows PFU/mL from a plaque assay as a function of nM lipid nanoparticle formulation for either 6 or 24 hour treatment on VeroE6 cells with the LNP formulation containing siRNA (SEQ ID NO: 141/142). VeroE6 cells were transfected by 0.1 nM to 300 nM of the LNP formulation containing the siRNA for 6 hours and 24 hours and then infected by SARS-CoV-2 at a MOI of 0.5.

FIG. 21 shows percent SARS-CoV-2 protein expression as a function of the concentration of siRNA (SEQ ID NO: 141/142) administered through a lipid nanoparticle. Vero-E6 cells were transfected with 0.1 nM to 300 nM concentration range of the LNP formulation containing the siRNA for 6 and 24 hours and then infected with SARS-CoV-2 isolate USA-WA1/2020 at a MOI of 0.5 for 24 hours. Total RNA was isolated and real-time PCR analysis performed using probes against the spike protein gene.

### DETAILED DESCRIPTION

Disclosed herein are siRNA compositions that specifically target Coronavirus proteins, and methods of using the compositions to treat, ameliorate, inhibit or prevent a Coronavirus protein-mediated disease or a symptom or condition related thereto, such as MERS, SARS or Covid-19.

Those skilled in the art recognize that MERS-CoV is the beta coronavirus that causes MERS, SARS-CoV is the beta coronavirus that causes SARS, and SARS-CoV-2 is the coronavirus that causes Covid-19. Reference herein to a Coronavirus will be understood to include MERS-CoV, SARS-CoV and SARS-CoV-2. Likewise, reference herein to a Coronavirus protein will be understood to include MERS-CoV proteins, SARS-CoV proteins and SARS-CoV-2 proteins.

A substantial amount of information is known about Coronaviruses. For example, the genome of SARS-CoV-2 contains more than 29,000 bases and encodes about 29 proteins. Four of the proteins have been identified as structural. The E and M proteins form a viral envelope. The N protein binds to the RNA genome SARS-CoV-2 and the S protein is capable of binding to receptors in the human lung. Other proteins are believed to be nonstructural, including the primary protease (Nsp5) and RNA polymerase (Nsp12). It is further believed that SARS-CoV-2 utilizes the ACE2 receptor to enter cells. In human subjects these receptors are present on nasal, lung, kidney, heart, and gut cells.

This disclosure includes compounds, compositions and methods for nucleic acid-based therapeutics for modulating expression of a Coronavirus. In some embodiments, this disclosure provides molecules active in RNA interference, as well as, structures and compositions that can silence expression of a Coronavirus gene. The structures and compositions of this disclosure can be used in preventing or treating or inhibiting various Coronavirus protein-mediated diseases such as MERS, SARS and/or Covid-19 or symptoms or conditions related thereto.

In further embodiments, this disclosure provides compositions for delivery and uptake of one or more therapeutic RNAi molecules or dsRNAs of this disclosure, as well as, methods of use thereof. The RNA-based compositions of this disclosure can be used in methods for preventing or treating respiratory diseases, such as MERS, SARS and/or Covid-19 or symptoms or conditions related thereto. As used herein, “RNAi molecules” refers to inhibitory dsRNAs and siRNAs, and in some contexts, “dsRNA” encompasses some RNAi molecules and siRNAs, and in some contexts, “siRNA” encompasses some dsRNAs and RNAi molecules. In some instances, these terms are used interchangeably.

Therapeutic compositions of this disclosure include nucleic acid molecules that are active in RNA interference. The therapeutic nucleic acid molecules can be targeted to a Coronavirus gene and are capable of or configured for gene silencing. In various embodiments, this disclosure provides a range of molecules that can be active as a small interfering RNA (siRNA) and can regulate, inhibit, reduce, or silence Coronavirus gene expression. The siRNAs of this disclosure are preferably used for preventing, inhibiting, ameliorating, or treating MERS, SARS and/or Covid-19 or a symptom or condition related thereto.

Embodiments of this disclosure further provide a vehicle, formulation, or lipid nanoparticle formulation for delivery of the inventive siRNAs described herein to subjects in need of preventing, inhibiting, or treating MERS, SARS and/or Covid-19 or a symptom or condition related thereto. This disclosure further contemplates methods for administering these siRNAs and compositions as therapeutics to mammals, such as humans.

The therapeutic molecules and compositions of this disclosure can be used for RNA interference directed to preventing, inhibiting, reducing, or treating an Coronavirus protein-associated disease, by administering a compound or composition described herein to a subject in need. The methods of this disclosure can utilize the inventive compounds for preventing or treating or inhibiting a lung disease such as MERS, SARS and/or Covid-19, for example.

In certain embodiments, a combination of therapeutic molecules of this disclosure can be used for silencing or inhibiting Coronavirus protein expression. This disclosure provides a range of siRNAs or RNAi molecules, each having a polynucleotide sense strand and a polynucleotide antisense strand; each strand of the molecule is from 15 to 30 nucleotides in length; a contiguous region of from 15 to 30 nucleotides of the antisense strand is complementary to a sequence of an mRNA encoding a Coronavirus protein; and at least a portion of the sense strand is complementary to at least a portion of the antisense strand, and the molecule has a duplex region of from 15 to 30 nucleotides in length.

An siRNA or RNAi molecule of this disclosure can have a contiguous region of from 15 to 30 nucleotides of the antisense strand that is complementary to a sequence of an mRNA encoding a Coronavirus protein, which is located in the duplex region of the molecule. In some embodiments the contiguous region of the antisense strand that is complementary to the sequence of the Coronavirus protein mRNA comprises or consists of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, nucleotides, or an amount of contiguous nucleotides of the mRNA of the Coronavirus protein that is within a range of nucleotides defined by any two of the aforementioned numbers of nucleotides. In some embodiments, an RNAi molecule or siRNA can have a contiguous region of from 15 to 30 nucleotides of the antisense strand that is complementary to a sequence of an mRNA encoding a Coronavirus protein.

Embodiments of this disclosure may further provide methods for preventing, treating, inhibiting or ameliorating one or more symptoms of a lung disease such as MERS, SARS and/or Covid-19, or reducing the risk of developing a lung disease such as MERS, SARS and/or Covid-19, or delaying the onset of a lung disease such as MERS, SARS and/or Covid-19, in a mammal in need thereof.

### Structures of Lipid Tails

Embodiments of a lipid-like compound of this disclosure may have one or more lipophilic tails that contain one or more alkyl or alkenyl groups. Examples of lipophilic tails include C(14:1(5))alkenyl, C(14:1(9))alkenyl, C(16:1(7))alkenyl, C(16:1(9))alkenyl, C(18:1(3))alkenyl, C(18:1(5))alkenyl, C(18:1(7))alkenyl, C(18:1(9))alkenyl, C(18:1(11))alkenyl, C(18:1(12))alkenyl, C(18:2(9,12))alkenyl, C(18:2(9,11))alkenyl, C(18:3(9,12,15))alkenyl, C(18:3(6,9,12))alkenyl, C(18:3(9,11,13))alkenyl, C(18:4(6,9,12,15))alkenyl, C(18:4(9,11,13,15))alkenyl, C(20:1(9))alkenyl, C(20:1(11))alkenyl, C(20:2(8,11))alkenyl, C(20:2(5,8))alkenyl, C(20:2(11,14))alkenyl, C(20:3(5,8,11))alkenyl, C(20:4(5,8,11,14))alkenyl, C(20:4(7,10,13,16))alkenyl, C(20:5(5,8,11,14,17))alkenyl, C(20:6(4,7,10,13,16,19))alkenyl, C(22:1(9))alkenyl, C(22:1(13))alkenyl, and C(24:1(9))alkenyl.

### Chemical Definitions

The term “alkyl” as used herein refers to a hydrocarbyl radical of a saturated aliphatic group, which can be of any length. An alkyl group can be a branched or unbranched, substituted or unsubstituted aliphatic group containing from 1 to 22 carbon atoms. This definition also applies to the alkyl portion of other groups such as, for example, cycloalkyl, alkoxy, alkanoyl, and aralkyl, for example.

As used herein, a term such as “C(1-5)alkyl” includes C(1)alkyl, C(2)alkyl, C(3)alkyl, C(4)alkyl, and C(5)alkyl. Likewise, for example, the term “C(3-22)alkyl” includes C(1)alkyl, C(2)alkyl, C(3)alkyl, C(4)alkyl, C(5)alkyl, C(6)alkyl, C(7)alkyl, C(8)alkyl, C(9)alkyl, C(10)alkyl, C(11)alkyl, C(12)alkyl, C(13)alkyl, C(14)alkyl, C(15)alkyl, C(16)alkyl, C(17)alkyl, C(18)alkyl, C(19)alkyl, C(20)alkyl, C(21)alkyl, and C(22)alkyl.

As used herein, an alkyl group may be designated by a term such as Me (methyl), Et (ethyl), Pr (any propyl group), ⁿPr (n-Pr, n-propyl), ⁱPr (i-Pr, isopropyl), Bu (any butyl group), ⁿBu (n-Bu, n-butyl), ⁱBu (i-Bu, isobutyl), ^{s}Bu (s-Bu, sec-butyl), and ^{t}Bu (t-Bu, tert-butyl).

The term “alkenyl” as used herein refers to hydrocarbyl radical having at least one carbon-carbon double bond. An alkenyl group can be branched or unbranched, substituted or unsubstituted hydrocarbyl radical having 2 to 22 carbon atoms and at least one carbon-carbon double bond.

The term “substituted” as used herein refers to an atom having one or more substitutions or substituents which can be the same or different and may include a hydrogen substituent. Thus, the terms alkyl, cycloalkyl, alkenyl, alkoxy, alkanoyl, and aryl, for example, refer to groups which can include substituted variations. Substituted variations include linear, branched, and cyclic variations, and groups having a substituent or substituents replacing one or more hydrogens attached to any carbon atom of the group.

In general, a compound may contain one or more chiral centers. Compounds containing one or more chiral centers may include those described as an “isomer,” a “stereoisomer,” a “diastereomer,” an “enantiomer,” an “optical isomer,” or as a “racemic mixture.” Conventions for stereochemical nomenclature, for example the stereoisomer naming rules of Cahn, Ingold and Prelog, as well as methods for the determination of stereochemistry and the separation of stereoisomers are known in the art. See, for example, Michael B. Smith and Jerry March, March's Advanced Organic Chemistry, 5th edition, 2001. The compounds and structures of this disclosure are meant to encompass all possible isomers, stereoisomers, diastereomers, enantiomers, and/or optical isomers that would be understood to exist for the specified compound or structure, including any mixture, racemic or otherwise, thereof.

This disclosure encompasses any and all tautomeric, solvated or unsolvated, hydrated or unhydrated forms, as well as any atom isotope forms of the compounds and compositions disclosed herein.

This disclosure encompasses any and all crystalline polymorphs or different crystalline forms of the compounds and compositions disclosed herein.

Coronaviruses and siRNAs

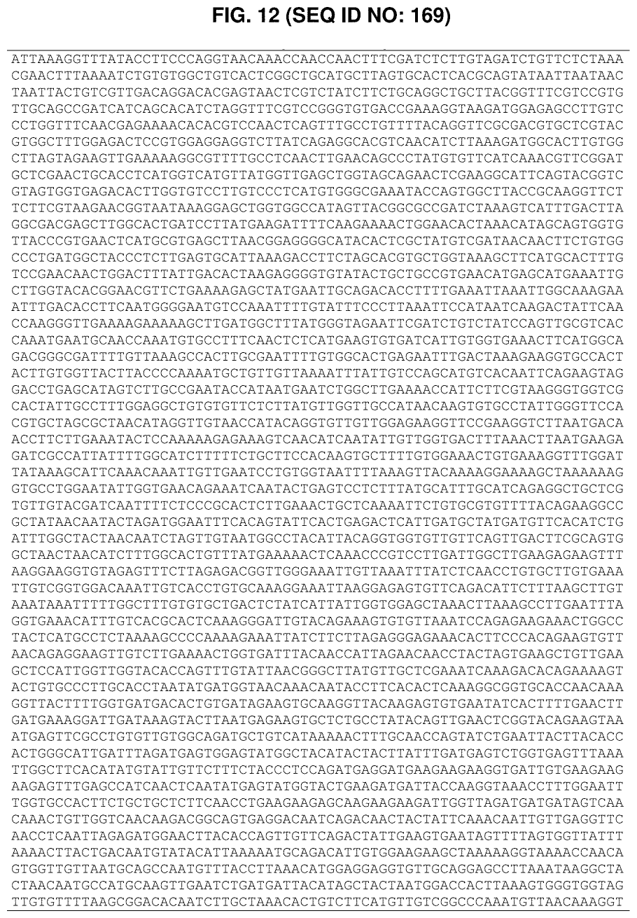

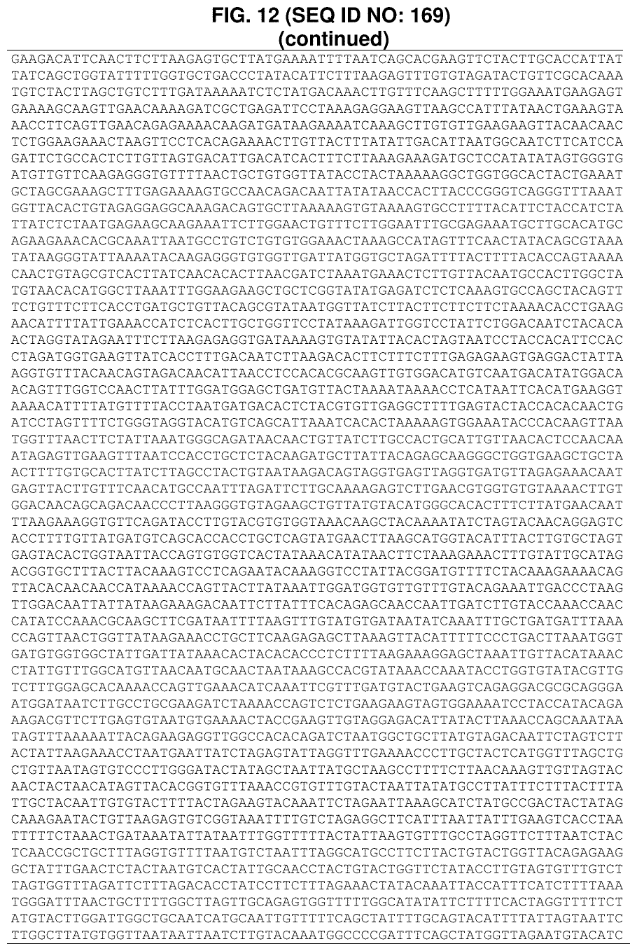

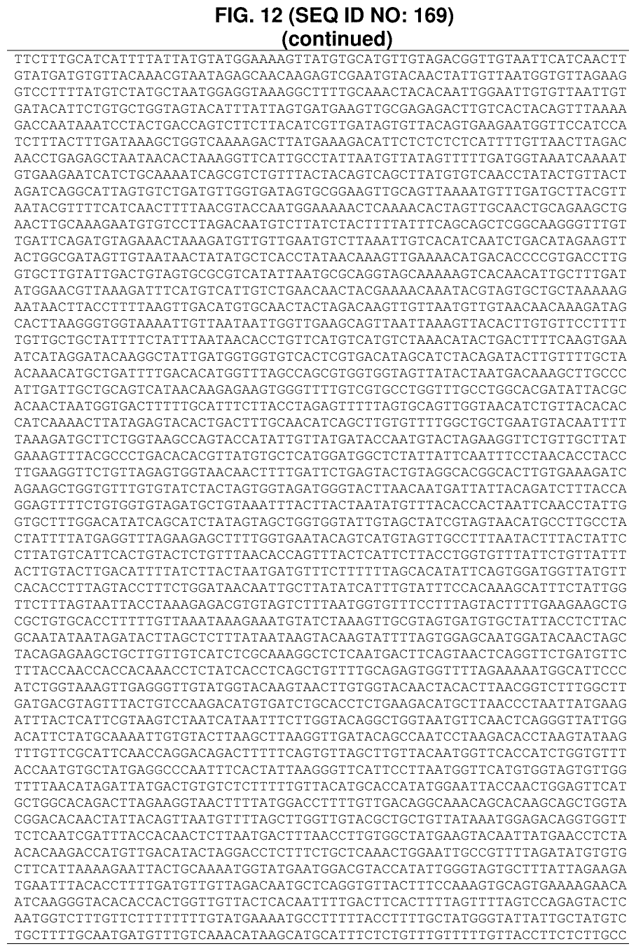

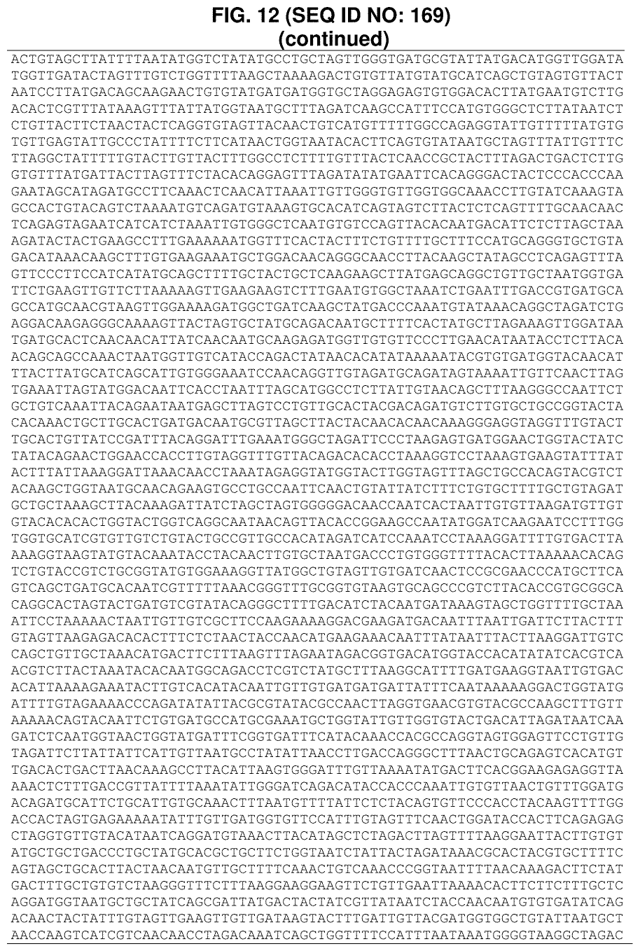

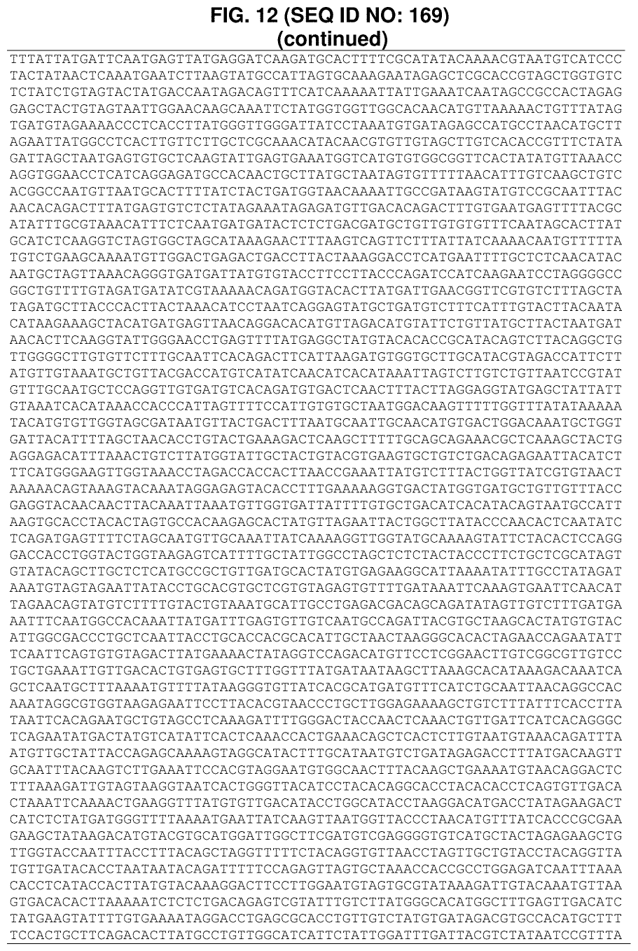

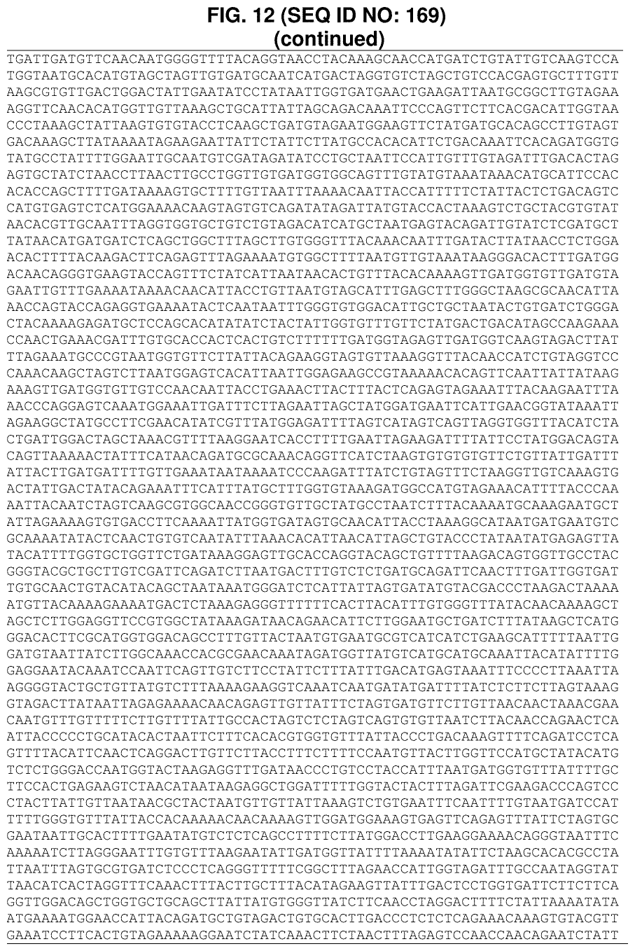

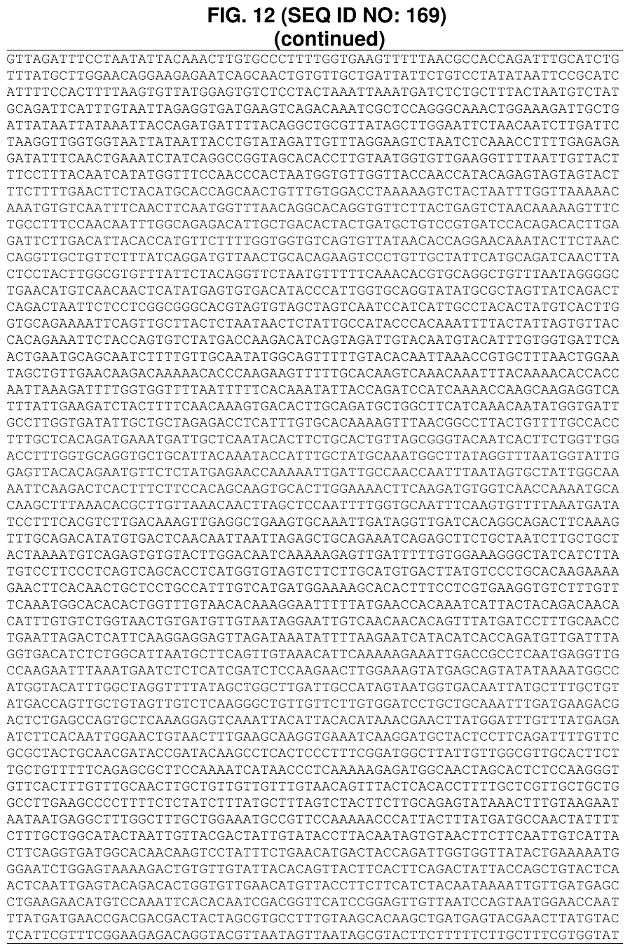

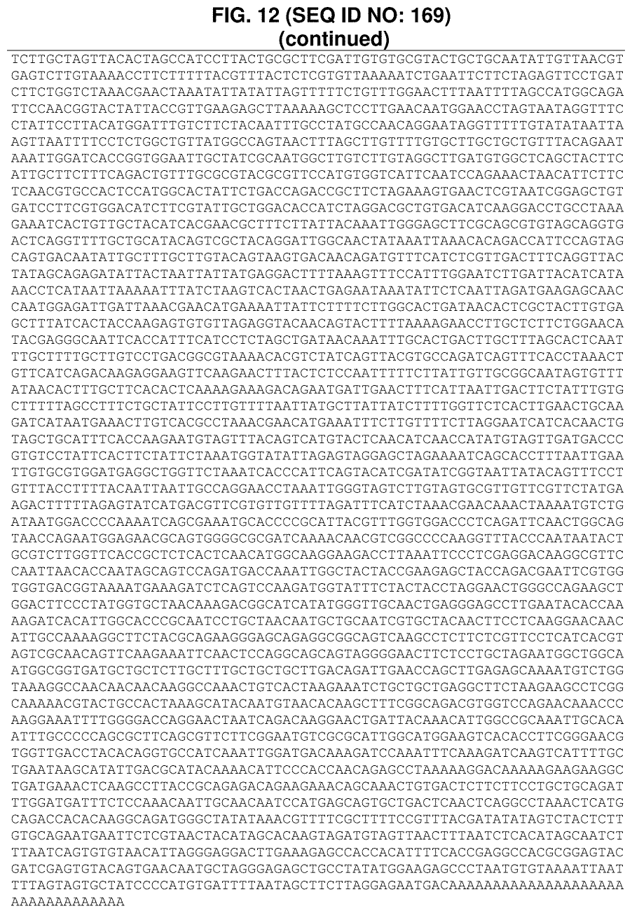

A nucleic acid sequence of the complete genome of an example of SARS-CoV-2 is disclosed in GenBank accession number NC_045512.2, and has the sequence shown in FIG. 12. This example of SARS-CoV-2 has a number of gene features, including those summarized in the following Table 1:

TABLE 1

| Version | Features | Start | End | Length | Gene Name | Product |
|---|---|---|---|---|---|---|
| NC_045512.2 | 5′UTR | 1 | 265 | 265 | 5UTR | |
| NC_045512.2 | mat_peptide | 266 | 805 | 540 | orflab | leader protein |
| NC_045512.2 | mat_peptide | 806 | 2719 | 1914 | orflab | nsp2 |
| NC_045512.2 | mat_peptide | 2720 | 8554 | 5835 | orflab | nsp3 |
| NC_045512.2 | mat_peptide | 8555 | 10054 | 1500 | orflab | nsp4 |
| NC_045512.2 | mat_peptide | 10055 | 10972 | 918 | orflab | 3C-like proteinase |
| NC_045512.2 | mat_peptide | 10973 | 11842 | 870 | orflab | nsp6 |
| NC_045512.2 | mat_peptide | 11843 | 12091 | 249 | orflab | nsp7 |
| NC_045512.2 | mat_peptide | 12092 | 12685 | 594 | orflab | nsp8 |
| NC_045512.2 | mat_peptide | 12686 | 13024 | 339 | orflab | nsp9 |
| NC_045512.2 | mat_peptide | 13025 | 13441 | 417 | orflab | nsp10 |
| NC_045512.2 | mat_peptide | 13442 | 13480 | 39 | orflab | nsp11 |
| NC_045512.2 | mat_peptide | 13442 | 16236 | 2795 | orflab | RNA-dependent |
| | | | | | | RNA polymerase |
| NC_045512.2 | mat_peptide | 16237 | 18039 | 1803 | orflab | helicase |
| NC_045512.2 | mat_peptide | 18040 | 19620 | 1581 | orflab | 3′-to-5′ |
| | | | | | | exonuclease |
| NC_045512.2 | mat_peptide | 19621 | 20658 | 1038 | orflab | endoRNAse |
| NC_045512.2 | mat_peptide | 20659 | 21552 | 894 | orflab | 2′-O-ribose |
| | | | | | | methyltransferase |
| NC_045512.2 | gene | 266 | 21555 | 21290 | orflab | |
| NC_045512.2 | gap | 21553 | 21562 | 10 | gap | orflab/S |
| NC_045512.2 | gene | 21563 | 25384 | 3822 | S | spike glycoprotein |
| NC_045512.2 | gap | 25385 | 25392 | 8 | gap | S/ORF3a |
| NC_045512.2 | gene | 25393 | 26220 | 828 | ORF3a | |
| NC_045512.2 | gap | 26221 | 26244 | 24 | gap | ORF3a/E |
| NC_045512.2 | gene | 26245 | 26472 | 228 | E | |
| NC_045512.2 | gap | 26473 | 26522 | 50 | gap | E/M |
| NC_045512.2 | gene | 26523 | 27191 | 669 | M | |
| NC_045512.2 | gap | 27192 | 27201 | 10 | gap | M/ORF6 |
| NC_045512.2 | gene | 27202 | 27387 | 186 | ORF6 | |
| NC_045512.2 | gap | 27388 | 27393 | 6 | gap | ORF6/ORF7a |
| NC_045512.2 | gene | 27394 | 27759 | 366 | ORF7a | |
| NC_045512.2 | gene | 27756 | 27887 | 132 | ORF7b | |
| NC_045512.2 | gap | 27888 | 27893 | 6 | gap | ORF7b/ORF8 |
| NC_045512.2 | gene | 27894 | 28259 | 366 | ORF8 | |
| NC_045512.2 | gap | 28260 | 28273 | 14 | gap | ORF8/N |
| NC_045512.2 | gene | 28274 | 29533 | 1260 | N | |
| NC_045512.2 | gap | 29534 | 29557 | 24 | gap | N/ORF10 |
| NC_045512.2 | gene | 29558 | 29674 | 117 | ORF10 | |
| NC_045512.2 | 3′UTR | 29675 | 29903 | 229 | 3UTR | |

One of ordinary skill in the art would readily understand that in some embodiments, the thymines in SEQ ID NO: 169 or in another sequence disclosed herein, would be uracils, or vice versa. Other examples of the complete genomes of various examples of SARS-CoV-2 are disclosed in GenBank, including those having the accession numbers in the following Table 2:

TABLE 2

| | | | |
|---|---|---|---|
| MT121215.1 | MT263421.1 | MT263429.1 | MT039888.1 |
| MT123292.2 | MT019529.1 | MT246452.1 | MT044257.1 |
| MT246460.1 | MT019531.1 | MT258377.1 | MT106052.1 |
| MT263459.1 | MT226610.1 | MT258383.1 | MT106053.1 |
| MT263391.1 | MT246467.1 | MT259254.1 | MT106054.1 |
| MT263381.1 | MT251976.1 | MT263406.1 | MT118835.1 |
| MT246459.1 | MT258381.1 | MT093571.1 | MT159705.1 |
| MT251978.1 | MT163717.1 | MT246477.1 | MT159706.1 |
| MT246480.1 | MT246466.1 | MT263399.1 | MT159707.1 |
| MT263395.1 | MT259278.1 | MT258382.1 | MT159708.1 |
| MN908947.3 | MT259269.1 | MT246474.1 | MT159709.1 |
| MT039890.1 | MT007544.1 | MT259251.1 | MT159710.1 |
| MT049951.1 | MT246454.1 | MT263398.1 | MT159711.1 |
| MT135041.1 | MT258378.1 | MN994468.1 | MT159712.1 |
| MT135042.1 | MT258379.1 | MT019533.1 | MT159713.1 |
| MT135043.1 | MT259273.1 | MT246487.1 | MT159714.1 |
| MT135044.1 | MT263468.1 | MT259236.1 | MT159715.1 |
| MT163716.1 | MN975262.1 | MN985325.1 | MT159717.1 |
| MT163718.1 | MN996528.1 | MN988713.1 | MT159718.1 |
| MT163719.1 | MT123290.1 | MN994467.1 | MT159719.1 |
| LC529905.1 | MT192772.1 | MN997409.1 | MT159720.1 |
| MT246462.1 | MT019532.1 | MT020880.1 | MT159721.1 |
| MT263396.1 | MT192773.1 | MT020881.1 | MT159722.1 |
| LC528232.1 | MT258380.1 | MT027062.1 | MT184907.1 |
| LC528233.1 | MT019530.1 | MT027063.1 | MT184909.1 |
| MT263382.1 | MT246478.1 | MT027064.1 | MT184910.1 |
| MT184911.1 | MT152824.1 | MT259231.1 | MT263402.1 |
| MT184912.1 | LC534418.1 | MT259244.1 | MT246457.1 |
| MT184913.1 | MT259263.1 | MT246489.1 | MT246468.1 |
| MT123291.2 | MT263403.1 | MT259229.1 | MT263392.1 |
| MT262896.1 | MT246461.1 | MT259237.1 | MT246488.1 |
| MT262897.1 | MT246475.1 | MT259271.1 | MT259246.1 |
| MT262898.1 | MT126808.1 | MT246476.1 | MT259275.1 |
| MT262899.1 | MT263418.1 | MT259227.1 | MT263400.1 |
| MT262900.1 | MT251975.1 | MT263431.1 | MT263425.1 |
| MT262901.1 | LC534419.1 | MT263439.1 | MT263450.1 |
| MT262902.1 | MT246481.1 | MT263440.1 | MT263415.1 |
| MT262903.1 | MT246450.1 | MT192759.1 | MT251979.1 |
| MT262904.1 | MT263438.1 | MT263413.1 | MT259281.1 |
| MT262905.1 | MT263446.1 | MT251972.1 | MT263420.1 |
| MT262906.1 | MT123293.2 | MT259228.1 | MN938384.1 |
| MT262907.1 | MT246471.1 | MT093631.2 | MT263430.1 |
| MT262908.1 | MT251973.1 | MT246470.1 | MT259261.1 |
| MT262909.1 | MT259260.1 | MT044258.1 | MT263422.1 |
| MT262910.1 | MT263410.1 | MN996531.1 | MT263454.1 |
| MT262911.1 | MT263417.1 | MT246455.1 | MT240479.1 |
| MT262912.1 | MT263437.1 | MT259286.1 | MT259264.1 |
| MT262913.1 | MT066175.1 | MT263444.1 | MT262993.1 |
| MT262914.1 | MT066176.1 | MT263469.1 | MT188341.1 |
| MT262915.1 | MT246484.1 | MT263074.1 | MT263405.1 |
| MT262916.1 | MT276598.1 | MN996530.1 | MT039873.1 |
| MT263435.1 | MT246469.1 | MT012098.1 | MT259256.1 |
| MT276323.1 | MT263443.1 | MT263419.1 | MT263452.1 |
| MT276324.1 | MT246451.1 | MT263433.1 | MT263464.1 |
| MT276325.1 | MT259226.1 | MT263467.1 | MT259285.1 |
| MT276326.1 | MT259257.1 | MN996529.1 | MT263414.1 |
| MT276327.1 | MT263445.1 | MT251974.1 | MT263424.1 |
| MT276328.1 | MT263447.1 | MT259267.1 | MT192765.1 |
| MT276329.1 | MT263463.1 | MT263387.1 | MT251977.1 |
| MT276330.1 | MT066156.1 | MT050493.1 | MT263412.1 |
| MT276331.1 | MT159716.1 | MT276597.1 | MT263423.1 |
| MN988668.1 | MT246667.1 | MT246490.1 | MT246449.1 |
| MN988669.1 | MT246486.1 | MT233526.1 | MT246479.1 |
| MT259277.1 | MT263465.1 | MT246456.1 | MT263448.1 |
| MT263458.1 | MT259230.1 | MT263436.1 | MT263449.1 |
| MT184908.1 | MT263404.1 | MT188340.1 | MT259245.1 |
| MT039887.1 | MT263411.1 | MT251980.1 | MN996527.1 |
| MT259248.1 | MT263432.1 | MT246482.1 | MT246473.1 |
| MT246453.1 | MT263434.1 | MT253707.1 | MT263455.1 |
| MT259249.1 | MT188339.1 | MT253708.1 | MT259250.1 |
| MT263442.1 | MT233519.1 | MT253709.1 | MT263394.1 |
| MT246464.1 | MT233522.1 | MT253710.1 | MT263451.1 |
| MT259282.1 | MT233523.1 | MT259274.1 | MT259287.1 |
| MT263416.1 | MT198652.2 | MT263428.1 | MT259241.1 |
| MT259253.1 | MT253696.1 | MT263453.1 | MT259258.1 |
| MT072688.1 | MT253697.1 | MT259266.1 | MT259247.1 |
| MT246472.1 | MT253698.1 | MT246485.1 | MT259280.1 |
| MT263408.1 | MT253699.1 | MT263456.1 | MT259239.1 |
| MT263386.1 | MT253700.1 | MT246458.1 | MT259284.1 |
| MT263457.1 | MT253701.1 | MT263462.1 | |
| MT263383.1 | MT253702.1 | MT263390.1 | |
| MT263384.1 | MT253703.1 | MT259268.1 | |
| MT263441.1 | MT253704.1 | MT259243.1 | |
| MT263426.1 | MT253705.1 | MT259235.1 | |
| MT259252.1 | MT253706.1 | MT263388.1 | |

Embodiments of this disclosure can provide compositions and methods for gene silencing of Coronavirus expression using small nucleic acid molecules. Examples of nucleic acid molecules include molecules active in RNA interference (RNAi molecules), short interfering RNA (siRNA), double-stranded RNA (dsRNA), micro-RNA (miRNA), or short hairpin RNA (shRNA) molecules, as well as, DNA-directed RNAs (ddRNA), Piwi-interacting RNAs (piRNA), or repeat associated siRNAs (rasiRNA). Such molecules are capable of mediating RNA interference against Coronavirus gene expression.

Some embodiments relate to a double-stranded ribonucleic acid (dsRNA) suitable for inhibiting expression of Coronavirus. In some embodiments, the dsRNA includes a sense strand; and an antisense strand; wherein the strands form a duplex region, and wherein the sense strand or antisense strand comprises 15 contiguous nucleotides from any one of SEQ ID NOs: 1-168. In some embodiments of the dsRNA, the sense strand comprises a nucleic acid sequence in accordance with SEQ ID NO: 1, and wherein the antisense strand comprises a nucleic acid sequence in accordance with SEQ ID NO: 2. In some embodiments, the sense strand comprises a nucleic acid sequence in accordance with SEQ ID NO: 11, and wherein the antisense strand comprises a nucleic acid sequence in accordance with SEQ ID NO: 12. In some embodiments, the sense strand and/or the antisense strand dsRNA has one or more modified nucleotides, for example as described herein under the heading, “Modified Nucleotides.”

The nucleic acid molecules and methods of this disclosure may be used to down regulate the expression of genes that encode a Coronavirus protein. The compositions and methods of this disclosure can include one or more nucleic acid molecules, which, independently or in combination, can modulate or regulate the expression of a Coronavirus protein and/or genes encoding Coronavirus proteins, proteins and/or genes encoding Coronavirus associated with the maintenance and/or development of diseases, conditions or disorders associated with Coronavirus, such as MERS, SARS and/or Covid-19.

The compositions and methods of this disclosure are described with reference to exemplary sequences of Coronavirus proteins. A person of ordinary skill in the art would understand that various aspects and embodiments of the disclosure are directed to any related Coronavirus genes, sequences, or variants, such as homolog genes and transcript variants, and polymorphisms, including single nucleotide polymorphism (SNP) associated with any Coronavirus genes.

In some embodiments, the compositions and methods of this disclosure can provide a double-stranded short interfering nucleic acid (siRNA) molecule that downregulates the expression of a Coronavirus gene.

An RNAi molecule or siRNA of this disclosure can be targeted to Coronavirus and any homologous sequences, for example, using complementary sequences or by incorporating non-canonical base pairs, mismatches and/or wobble base pairs, that can provide additional target sequences.

In instances where mismatches are identified, non-canonical base pairs, for example, mismatches and/or wobble bases can be used to generate nucleic acid molecules that target more than one gene sequence.

For example, non-canonical base pairs such as UU and CC base pairs can be used to generate nucleic acid molecules that are capable of or are configured for targeting sequences for differing Coronavirus targets that share sequence homology. Thus, an RNAi molecule or siRNA can be targeted to a nucleotide sequence that is conserved between homologous genes, and a single RNAi molecule or siRNA can be used to inhibit expression of more than one gene.

In some aspects, the compositions and methods of this disclosure include RNAi molecules or siRNAs that are active against Coronavirus protein mRNA, wherein the RNAi molecule or siRNAs include a sequence complementary to any mRNA encoding a Coronavirus sequence.

In some embodiments, an RNAi molecule or siRNA of this disclosure can have activity against a Coronavirus protein RNA, wherein the RNAi molecule or siRNA includes a sequence complementary to an RNA having a variant Coronavirus encoding sequence, for example, a mutant Coronavirus gene known in the art to be associated with a lung disease such as MERS, SARS and/or Covid-19.

In further embodiments, an RNAi molecule or siRNA of this disclosure can include a nucleotide sequence that can interact with a nucleotide sequence of a Coronavirus gene and mediate silencing of Coronavirus gene expression. The nucleic acid molecules for inhibiting expression of Coronavirus may have a sense strand and an antisense strand, wherein the strands form a duplex region. The nucleic acid molecules may have one or more of the nucleotides in the duplex region being modified, including such nucleotide modifications as are known in the art. Any nucleotide in an overhang of the siRNA may also be modified.

In some embodiments, the preferred modified nucleotides are 2′-deoxy nucleotides. In additional embodiments, the modified nucleotides can include 2′-O-alkyl substituted nucleotides, 2 ‘-deoxy-2’-fluoro substituted nucleotides, phosphorothioate nucleotides, or locked nucleotides, or any combination thereof.

In some embodiments, the nucleic acid molecules of this disclosure can inhibit expression of a Coronavirus protein mRNA with an advantageous IC50 of less than about 300 pM, or less than about 200 pM, or less than about 100 pM, or less than about 50 pM. In some embodiments, the nucleic acid molecules can inhibit expression of a Coronavirus mRNA or protein levels by at least 10%, 25%, 50%, 75%, 85%, 90%, 95%, or 99% in vitro or in vivo, upon a single administration. In some embodiments, the nucleic acid molecules can inhibit expression of a Coronavirus mRNA or protein levels, in vitro or in vivo, by 10%, 25%, 50%, 75%, 85%, 90%, 95%, 99%, or 100%, or by a range of percentages defined by any two of the aforementioned percentages, upon administration. In some embodiments, the inhibition of a Coronavirus mRNA or protein expression occurs upon administration of 1, 2, 3, 4, 5, 6, 7, 8, 9, or 10 doses, or by a range of doses defined by any two of the aforementioned numbers. In some embodiments, the inhibition of a Coronavirus mRNA or protein expression occurs within 1, 2, 3, 4, 5, 6, 7, 8, 9, or 10 days, or a range of days defined by any two of the aforementioned numbers, following administration of one or more doses of the nucleic acid molecules. In some embodiments, the inhibition of a Coronavirus mRNA or protein expression is maintained for 1 day, 2 days, 3 days, 4 days, 5 days, 6 days, 7 days, 8 days, 9 days, 10 days, 2 weeks, 3 weeks, 4 weeks, 1 month, 2 months, 3 months, 4 months, 5 months, or 6 months, or for a range of time periods defined by any two of the aforementioned time periods, following administration of one or more doses of the nucleic acid molecules.

Pharmaceutical compositions are contemplated in this disclosure, which can contain one or more siRNAs as described herein, in combination with a pharmaceutically acceptable carrier. Any suitable carrier may be used, including those known in the art, as well as lipid molecules, nanoparticles, or liposomes, any of which may encapsulate the siRNA molecules.

This disclosure includes methods for treating or inhibiting a disease associated with Coronavirus expression, which methods include administering to a subject in need a composition containing one or more of the siRNAs described herein. Some embodiments relate to a method for inhibiting, preventing, or treating MERS, SARS and/or Covid-19, the method comprising administering to a subject in need, such as a human, a dsRNA, siRNA, pharmaceutical composition, vector, or cell as described herein. Diseases to be treated may include lung diseases such as MERS, SARS and/or Covid-19, as well as symptoms and associated conditions such as a cytokine storm triggered by MERS, SARS and/or Covid-19.

Some embodiments relate to a pharmaceutical composition comprising nanoparticles encapsulating an siRNA comprising a specific inhibitor or antagonist of a Coronavirus protein, as described herein. Some embodiments relate to use of the pharmaceutical composition for treating, preventing, inhibiting, or ameliorating MERS, SARS and/or Covid-19 or a symptom of MERS, SARS and/or Covid-19.

Examples of nucleic acids or RNAi molecules of this disclosure targeted to a Coronavirus mRNA are shown in Tables 3-5 below. The general structure of siRNA is that of two RNA strands forming a 19 bp long duplex, with 3′ dinucleotide overhangs (which may be abbreviated herein as “OH”) on each strand. The antisense strand is reverse complement of the target mRNA. Those skilled in the art will recognize that SEQ ID NOS: 1-60 correspond to SEQ ID NOS: 61-120, except that the siRNA of SEQ ID NOS: 61-120 include the indicated overhangs. Thus, it is apparent to those skilled in the art that the siRNA of SEQ ID NOS: 61-120 are modified versions with 3′ dinucleotide overhangs of the base siRNA of SEQ ID NOS: 1-60, respectively.

TABLE 3
SARS-CoV-2: Target sequences

| SEQ ID NO | Target | Strand | Nucleotide Sequence (5′ −> 3′) |
|---|---|---|---|
| 1 | RNA-dependent RNA polymerase | S | UCGUCAACAACCUAGACAA |
| 2 | | A | UUGUCUAGGUUGUUGACGA |
| 3 | RNA-dependent RNA polymerase | S | AAGAAUAGAGCUCGCACCG |
| 4 | | A | CGGUGCGAGCUCUAUUCUU |
| 5 | RNA-dependent RNA polymerase | S | AGAAUAGAGCUCGCACCGU |
| 6 | | A | ACGGUGCGAGCUCUAUUCU |
| 7 | RNA-dependent RNA polymerase | S | AGAGCCAUGCCUAACAUGC |
| 8 | | A | GCAUGUUAGGCAUGGCUCU |
| 9 | 3′-to-5′ exonuclease | S | AUCACCCGCGAAGAAGCUA |
| 10 | | A | UAGCUUCUUCGCGGGUGAU |
| 11 | 3′-to-5′ exonuclease | S | UCACCCGCGAAGAAGCUAU |
| 12 | | A | AUAGCUUCUUCGCGGGUGA |
| 13 | nsp3 | S | UGCUCACCUAUAACAAAGU |
| 14 | | A | ACUUUGUUAUAGGUGAGCA |
| 15 | RNA-dependent RNA polymerase | S | UAGCUGGUGUCUCUAUCUG |
| 16 | | A | CAGAUAGAGACACCAGCUA |
| 17 | RNA-dependent RNA polymerase | S | UCUCUAUCUGUAGUACUAU |
| 18 | | A | AUAGUACUACAGAUAGAGA |
| 19 | RNA-dependent RNA polymerase | S | CUCUAUCUGUAGUACUAUG |
| 20 | | A | CAUAGUACUACAGAUAGAG |
| 21 | RNA-dependent RNA polymerase | S | GAUGCCACAACUGCUUAUG |
| 22 | | A | CAUAAGCAGUUGUGGCAUC |
| 23 | helicase | S | GGUACUGGUAAGAGUCAUU |
| 24 | | A | AAUGACUCUUACCAGUACC |
| 25 | helicase | S | AUAGGUCCAGACAUGUUCC |
| 26 | | A | GGAACAUGUCUGGACCUAU |
| 27 | endoRNAse | S | AUAACAGAUGCGCAAACAG |
| 28 | | A | CUGUUUGCGCAUCUGUUAU |
| 29 | endoRNAse | S | UAACAGAUGCGCAAACAGG |
| 30 | | A | CCUGUUUGCGCAUCUGUUA |
| 31 | endoRANse | S | AGAUGCGCAAACAGGUUCA |
| 32 | | A | UGAACCUGUUUGCGCAUCU |
| 33 | E | S | ACACUAGCCAUCCUUACUG |
| 34 | | A | CAGUAAGGAUGGCUAGUGU |
| 35 | E | S | ACUAGCCAUCCUUACUGCG |
| 36 | | A | CGCAGUAAGGAUGGCUAGU |
| 37 | N | S | CAAUAAUACUGCGUCUUGG |
| 38 | | A | CCAAGACGCAGUAUUAUUG |
| 39 | N | S | AAUAGCAGUCCAGAUGACC |
| 40 | | A | GGUCAUCUGGACUGCUAUU |
| 41 | N | S | UUCUACUACCUAGGAACUG |
| 42 | | A | CAGUUCCUAGGUAGUAGAA |
| 43 | N | S | UGCCAAAAGGCUUCUACGC |
| 44 | | A | GCGUAGAAGCCUUUUGGCA |
| 45 | N | S | CAGAUUGAACCAGCUUGAG |
| 46 | | A | CUCAAGCUGGUUCAAUCUG |
| 47 | N | S | AGAUUGAACCAGCUUGAGA |
| 48 | | A | UCUCAAGCUGGUUCAAUCU |
| 49 | N | S | CUGGUAAAGGCCAACAACA |
| 50 | | A | UGUUGUUGGCCUUUACCAG |
| 51 | N | S | GGUAAAGGCCAACAACAAC |
| 52 | | A | GUUGUUGUUGGCCUUUACC |
| 53 | N | S | GGCCAAACUGUCACUAAGA |
| 54 | | A | UCUUAGUGACAGUUUGGUU |
| 55 | N | S | GCCAAACUGUCACUAAGAA |
| 56 | | A | UUCUUAGUGACAGUUUGGC |
| 57 | ORF10 | S | UUAAUCUCACAUAGCAAUC |
| 58 | | A | GAUUGCUAUGUGAGAUUAA |
| 59 | 3′UTR | S | GACUUGAAAGAGCCACCAC |
| 60 | | A | GUGGUGGCUCUUUCAAGUC |

Strand S = sense; strand A = antisense; nucleotides A, G, C and U refer to ribo-A,
ribo-G, ribo-C and ribo-U, respectively.

TABLE 4
Sense and antisense strands with mUmU overhangs

| SEQ ID NO | Target | Strand | Nucleotide Sequence (5′ −> 3′) |
|---|---|---|---|
| 61 | RNA-dependent | S | UCGUCAACAACCUAGACAAmUmU |
| 62 | RNA polymerase | A | UUGUCUAGGUUGUUGACGAmUmU |
| 63 | RNA-dependent | S | AAGAAUAGAGCUCGCACCGmUmU |
| 64 | RNA polymerase | A | CGGUGCGAGCUCUAUUCUUmUmU |
| 65 | RNA-dependent | S | AGAAUAGAGCUCGCACCGUmUmU |
| 66 | RNA polymerase | A | ACGGUGCGAGCUCUAUUCUmUmU |
| 67 | RNA-dependent | S | AGAGCCAUGCCUAACAUGCmUmU |
| 68 | RNA polymerase | A | GCAUGUUAGGCAUGGCUCUmUmU |
| 69 | 3′-to-5′ | S | AUCACCCGCGAAGAAGCUAmUmU |
| 70 | exonuclease | A | UAGCUUCUUCGCGGGUGAUmUmU |
| 71 | 3′-to-5′ | S | UCACCCGCGAAGAAGCUAUmUmU |
| 72 | exonuclease | A | AUAGCUUCUUCGCGGGUGAmUmU |
| 73 | nsp3 | S | UGCUCACCUAUAACAAAGUmUmU |
| 74 | | A | ACUUUGUUAUAGGUGAGCAmUmU |
| 75 | RNA-dependent | S | UAGCUGGUGUCUCUAUCUGmUmU |
| 76 | RNA polymerase | A | CAGAUAGAGACACCAGCUAmUmU |
| 77 | RNA-dependent | S | UCUCUAUCUGUAGUACUAUmUmU |
| 78 | RNA polymerase | A | AUAGUACUACAGAUAGAGAmUmU |
| 79 | RNA-dependent | S | CUCUAUCUGUAGUACUAUGmUmU |
| 80 | RNA polymerase | A | CAUAGUACUACAGAUAGAGmUmU |
| 81 | RNA-dependent | S | GAUGCCACAACUGCUUAUGmUmU |
| 82 | RNA polymerase | A | CAUAAGCAGUUGUGGCAUCmUmU |
| 83 | helicase | S | GGUACUGGUAAGAGUCAUUmUmU |
| 84 | | A | AAUGACUCUUACCAGUACCmUmU |
| 85 | helicase | S | AUAGGUCCAGACAUGUUCCmUmU |
| 86 | | A | GGAACAUGUCUGGACCUAUmUmU |
| 87 | endoRNAse | S | AUAACAGAUGCGCAAACAGmUmU |
| 88 | | A | CUGUUUGCGCAUCUGUUAUmUmU |
| 89 | endoRNAse | S | UAACAGAUGCGCAAACAGGmUmU |
| 90 | | A | CCUGUUUGCGCAUCUGUUAmUmU |
| 91 | endoRNAse | S | AGAUGCGCAAACAGGUUCAmUmU |
| 92 | | A | UGAACCUGUUUGCGCAUCUmUmU |
| 93 | E | S | ACACUAGCCAUCCUUACUGmUmU |
| 94 | | A | CAGUAAGGAUGGCUAGUGUmUmU |
| 95 | E | S | ACUAGCCAUCCUUACUGCGmUmU |
| 96 | | A | CGCAGUAAGGAUGGCUAGUmUmU |
| 97 | N | S | CAAUAAUACUGCGUCUUGGmUmU |
| 98 | | A | CCAAGACGCAGUAUUAUUGmUmU |
| 99 | N | S | AAUAGCAGUCCAGAUGACCmUmU |
| 100 | | A | GGUCAUCUGGACUGCUAUUmUmU |
| 101 | N | S | UUCUACUACCUAGGAACUGmUmU |
| 102 | | A | CAGUUCCUAGGUAGUAGAAmUmU |
| 103 | N | S | UGCCAAAAGGCUUCUACGCmUmU |
| 104 | | A | GCGUAGAAGCCUUUUGGCAmUmU |
| 105 | N | S | CAGAUUGAACCAGCUUGAGmUmU |
| 106 | | A | CUCAAGCUGGUUCAAUCUGmUmU |
| 107 | N | S | AGAUUGAACCAGCUUGAGAmUmU |
| 108 | | A | UCUCAAGCUGGUUCAAUCUmUmU |
| 109 | N | S | CUGGUAAAGGCCAACAACAmUmU |
| 110 | | A | UGUUGUUGGCCUUUACCAGmUmU |
| 111 | N | S | GGUAAAGGCCAACAACAACmUmU |
| 112 | | A | GUUGUUGUUGGCCUUUACCmUmU |
| 113 | N | S | GGCCAAACUGUCACUAAGAmUmU |
| 114 | | A | UCUUAGUGACAGUUUGGCCmUmU |
| 115 | N | S | GCCAAACUGUCACUAAGAAmUmU |
| 116 | | A | UUCUUAGUGACAGUUUGGCmUmU |
| 117 | ORF10 | S | UUAAUCUCACAUAGCAAUCmUmU |
| 118 | | A | GAUUGCUAUGUGAGAUUAAmUmU |
| 119 | 3′UTR | S | GACUUGAAAGAGCCACCACmUmU |
| 120 | | A | GUGGUGGCUCUUUCAAGUCmUmU |

Strand S = sense; strand A = antisense; nucleotides A, G, C and U refer to
ribo-A, ribo-G, ribo-C and ribo-U, respectively; mU refers to 2′-OMe-modified U.

TABLE 5
Modified Sense and antisense strands with mUmU overhangs

| SEQ ID NO | Target | Strand | Nucleotide Sequence (5′ −> 3′) |
|---|---|---|---|
| 121 | RNA-dependent | S | UCGUCAACAACCmUAGAmCAAmUmU |
| 122 | RNA polymerase | A | UUGUCmUAGGUUGUUGACGAmUmU |
| 123 | RNA-dependent | S | UmCGUCAACAACmCmUAGAmCAAmUmU |
| 124 | RNA polymerase | A | mUmUGmUCmUAGGUUGUUGACGAmUmU |
| 125 | RNA-dependent | S | UCGUmCAAmCAACCmUAGAmCAAmUmU |
| 126 | RNA polymerase | A | UUGUCmUAGGUUGUUGACGAmUmU |
| 127 | RNA-dependent | S | UCGUmCAAmCAACCmUAGAmCAAmUmU |
| 128 | RNA polymerase | A | mUmUGmUCmUAGGUUGUUGACGAmUmU |
| 129 | RNA-dependent | S | mUmCGmUmCAAmCAAmCmCmUAGAmCAAmUmU |
| 130 | RNA polymerase | A | UUGUCmUAGGUUGUUGACGAmUmU |
| 131 | RNA-dependent | S | mUmCGmUmCAAmCAAmCmCmUAGAmCAAmUmU |
| 132 | RNA polymerase | A | mUmUGmUCmUAGGUUGUUGACGAmUmU |
| 133 | RNA-dependent | S | UCGUmCAAmCAACCmUAGAmCAAmUmU |
| 134 | RNA polymerase | A | UUgUcmUaGGUUGUUGACGAmUmU |
| 135 | RNA-dependent | S | UCGUmCAAmCAACCmUAGAmCAAmUmU |
| 136 | RNA polymerase | A | UUGuCmUAgGUUGUUGACGAmUmU |
| 137 | RNA-dependent | S | UCGUmCAAmCAACCmUAGAmCAAmUmU |
| 138 | RNA polymerase | A | mUmUgmUcmUaGGUUGUUGACGAmUmU |
| 139 | RNA-dependent | S | UmCGUCAACAACmCmUAGAmCAAmUmU |
| 140 | RNA polymerase | A | UUgUcmUaGGUUGUUGACGAmUmU |
| 141 | RNA-dependent | S | UmCGUCAACAACmCmUAGAmCAAmUmU |
| 142 | RNA polymerase | A | UUGuCmUAgGUUGUUGACGAmUmU |
| 143 | RNA-dependent | S | UmCGUCAACAACmCmUAGAmCAAmUmU |
| 144 | RNA polymerase | A | mUmUgmUcmUaGGUUGUUGACGAmUmU |
| 145 | 3′-to-5′ | S | UCACCCGCGAAGAAGCmUAUmUmU |
| 146 | exonuclease | A | AmUAGCUUCUUCGCGGGUGAmUmU |
| 147 | 3′-to-5′ | S | UCACCCGCGAAGAmAmGmCmUAUmUmU |
| 148 | exonuclease | A | AmUAGCUUCUmUCGCGmGGUGAmUmU |
| 149 | 3′-to-5′ | S | UmCACCCGCGAAGAAGCmUAUmUmU |
| 150 | exonuclease | A | AmUAGCUUCUUCGCGGGUGAmUmU |
| 151 | 3′-to-5′ | S | UmCACCCGCGAAGAAGCmUAUmUmU |
| 152 | exonuclease | A | AmUAGCUUCUmUCGCGmGGUGAmUmU |
| 153 | 3′-to-5′ | S | mUmCAmCmCmCGmCGAAGAAGmCmUAmUmUmU |
| 154 | exonuclease | A | AmUAGCUUCUUCGCGGGUGAmUmU |
| 155 | 3′-to-5′ | S | mUmCAmCmCmCGmCGAAGAAGmCmUAmUmUmU |
| 156 | exonuclease | A | AmUAGCUUCUmUCGCGmGGUGAmUmU |
| 157 | 3′-to-5′ | S | UmCACCCGCGAAGAAGCmUAUmUmU |
| 158 | exonuclease | A | AmUaGcUuCUUCGCGGGUGAmUmU |
| 159 | 3′-to-5′ | S | UmCACCCGCGAAGAAGCmUAUmUmU |
| 160 | exonuclease | A | AmUAgCuUcUUCGCGGGUGAmUmU |
| 161 | 3′-to-5′ | S | UmCACCCGCGAAGAAGCmUAUmUmU |
| 162 | exonuclease | A | AmUaGcUuCUmUCGCGmGGUGAmUmU |
| 163 | 3′-to-5′ | S | UCACCCGCGAAGAmAmGmCmUAUmUmU |
| 164 | exonuclease | A | AmUaGcUuCUUCGCGGGUGAmUmU |
| 165 | 3′-to-5′ | S | UCACCCGCGAAGAmAmGmCmUAUmUmU |
| 166 | exonuclease | A | AmUAgCuUcUUCGCGGGUGAmUmU |
| 167 | 3′-to-5′ | S | UCACCCGCGAAGAmAmGmCmUAUmUmU |
| 168 | exonuclease | A | AmUaGcUuCUmUCGCGmGGUGAmUmU |

Strand S = sense; strand A = antisense; nucleotides A, G, C and U refer to
ribo-A, ribo-G, ribo-C and ribo-U, respectively; a, g, c and u refer to
2-deoxy-A, 2-deoxy-G, 2-deoxy-C and 2-deoxy-U, respectively; and mA, mG, mC
and mU refer to 2′OMe-modified A, 2′OMe-modified G, 2′OMe-modified C and
2′OMe-modified U, respectively.

This disclosure includes a range of nucleic acid molecules, wherein: a) the molecule has a polynucleotide sense strand and a polynucleotide antisense strand; b) each strand of the molecule is from 15 to 30 nucleotides in length; c) a contiguous region of from 15 to 30 nucleotides of the antisense strand is complementary to a sequence of an mRNA encoding a Coronavirus protein; d) at least a portion of the sense strand is complementary to at least a portion of the antisense strand, or the molecule has a duplex region of from 15 to 30 nucleotides in length or, preferably, the nucleic acid molecules have all of the features of (a)-(d).

In some embodiments, the nucleic acid molecule can have a contiguous region of from 15 to 30 nucleotides of the antisense strand that is complementary to a sequence of an mRNA encoding a Coronavirus protein and which is located in the duplex region of the molecule. In additional embodiments, the nucleic acid molecule can have a contiguous region of from 15 to 30 nucleotides of the antisense strand that is complementary to a sequence of an mRNA encoding a Coronavirus protein.

In certain embodiments, each strand of the nucleic acid molecule is from 18 to 22 nucleotides in length. The duplex region of the nucleic acid molecule can be 19 nucleotides in length. In some embodiments, each strand or the duplex region comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or more, nucleotides, or an amount of nucleotides that is within a range of nucleotides defined by any two of the aforementioned numbers of nucleotides.

In alternative forms, the nucleic acid molecule can have a polynucleotide sense strand and a polynucleotide antisense strand that are connected as a single strand, and form a duplex region connected at one end by a loop.

Some embodiments of a nucleic acid molecule of this disclosure can have a blunt end. In certain embodiments, a nucleic acid molecule can have one or more 3′ overhangs.

This disclosure provides a range of nucleic acid molecules that are RNAi molecules, or siRNAs, active for gene silencing or for reducing gene expression. The nucleic acid molecules can be a dsRNA, an siRNA, a micro-RNA, or a shRNA active for gene silencing, as well as, a DNA-directed RNA (ddRNA), Piwi-interacting RNA (piRNA), or a repeat associated siRNA (rasiRNA). The nucleic acid molecules can be active for inhibiting expression of a Coronavirus protein.

Embodiments of this disclosure further provide nucleic acid molecules having an IC50 for knockdown of a Coronavirus protein of less than 100 pM. Additional embodiments of this disclosure provide nucleic acid molecules having an IC50 for knockdown of a Coronavirus protein of less than 50 pM. In some embodiments of the dsRNA or siRNA, the dsRNA or siRNA inhibits expression of a Coronavirus protein mRNA in lung cells, or other cells such as nasal cells, with an EC50 of less than 1000, 500, 250, 100, 75, 50, or 25 pM, or an EC50 that is within a range defined by any two of the aforementioned numbers. In some embodiments, the dsRNA or siRNA decreases or prevents expression of a Coronavirus protein in a lung cell in vivo.

This disclosure further contemplates compositions containing one or more nucleic acid molecules, along with a pharmaceutically acceptable carrier. In certain embodiments, the carrier can be a lipid molecule or liposome or nanoparticle. Some embodiments of the compounds and compositions of this disclosure are useful in methods for preventing, inhibiting or treating a Coronavirus associated disease, or symptom or condition related thereto by administering a compound or composition to a subject in need such as a human.

Some embodiments relate to a dsRNA agent or siRNA agent suitable for inhibiting expression of a Coronavirus protein. In some embodiments, the dsRNA or siRNA includes a sense strand and an antisense strand, the antisense strand comprising a region of complementarity, which comprises at least 15 contiguous nucleotides from the nucleotide sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168.

Some embodiments relate to a sense or antisense polynucleotide agent for inhibiting expression of a Coronavirus protein. In some embodiments, the agent includes 4 to 50, or about 4 to about 50, contiguous nucleotides, wherein at least one of the contiguous nucleotides is a modified nucleotide, and wherein the nucleotide sequence of the agent is at least 80%, 81%, 82%, 83%, 84%, 85%, 86%, 87%, 88%, 89%, 90%, 91%, 92%, 93%, 94%, 95%, 96%, 97%, 98%, 99%, or 100%, or within a range defined by any two of the aforementioned percentages, complementary over its entire length to the equivalent region of the nucleotide sequence of any one of SEQ ID NOs: 1-168.

Some embodiments relate to a nucleic acid molecule or siRNA for inhibiting expression of a Coronavirus protein. In some embodiments, the nucleic acid molecule or siRNA includes a sense strand and an antisense strand; wherein the strands form a duplex region; and wherein the sense strand and the antisense strand each have a sequence in accordance with any one of SEQ ID NOs: 1-60. In some embodiments, the nucleic acid molecule or siRNA includes a sense strand and an antisense strand; wherein the strands form a duplex region; and wherein the sense strand and the antisense strand each have a sequence in accordance with any one of SEQ ID NOs: 61-120.

Some embodiments relate to a nucleic acid molecule or siRNA for inhibiting expression of a Coronavirus protein. In some embodiments, the nucleic acid molecule or siRNA includes a sense strand and an antisense strand; wherein the strands form a duplex region; and wherein the sense strand and the antisense strand each have a sequence in accordance with any one of SEQ ID NOs: 121-168.

### Modified Nucleotides

Embodiments of this disclosure encompass siRNA molecules include a modified nucleotide to provide enhanced properties for therapeutic use, such as increased activity and potency for Coronavirus gene silencing. This disclosure provides siRNA molecules with one or more modified nucleotides that can have increased serum stability, as well as reduced off target effects, without loss of activity and potency of the siRNA molecules for gene modulation and gene silencing. In some aspects, this disclosure provides siRNAs having nucleotide modifications in various combinations, which enhance the stability and efficacy of the siRNA.

In some embodiments of the dsRNA, siRNA, or RNAi molecules, one or more nucleotides in the duplex region is modified. In some embodiments, the last 2 nucleotides on the 3′ end of the sense strand are modified nucleotides. In some embodiments, 1, 2, 3, 4 or 5, or a range defined by any of the two aforementioned numbers, of the last 5 nucleotides on the 3′ or 5′ end or both of the antisense strand are modified nucleotides. In some embodiments, the modified nucleotides are selected from 2′-deoxy nucleotides, 2′-O-alkyl substituted nucleotides, 2′-deoxy-2′-fluoro substituted nucleotides, phosphorothioate nucleotides, or locked nucleotides, or any combination thereof. Some embodiments include a 2′-deoxy-2′-fluoro substituted nucleotide in the duplex region.

Some embodiments relate to a dsRNA for inhibiting expression of a Coronavirus protein, comprising a sense strand and an antisense strand comprising a region of complementarity complementary to an mRNA encoding a Coronavirus protein, wherein each strand is at least 15 nucleotides in length, wherein the strands form a duplex region, wherein the dsRNA comprises at least one modified nucleotide, wherein said modified nucleotide is selected from the group of: a 2′-O-methyl modified nucleotide, a nucleotide comprising a 5′-phosphorothioate group, a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural base comprising nucleotide, and wherein the region of complementarity of the antisense strand has a 2′-deoxy-modified nucleotide in a plurality of positions.

In some embodiments, the siRNA molecules of this disclosure can have passenger strand off target activity reduced by at least 10-fold, or at least 20-fold, or at least 30-fold, or at least 50-fold, or at least 100-fold.

As used herein, the term “modified” refers to one or more changes made in the structure of a naturally-occurring nucleotide or nucleic acid structure of an siRNA, which encompasses siRNAs having one or more nucleotide analogs, altered nucleotides, non-standard nucleotides, non-naturally occurring nucleotides, and combinations thereof.

In some embodiments, the number of modified structures in an siRNA can include all of the structural components, and/or all of the nucleotides of the siRNA molecule.

Examples of siRNAs with modified nucleotides include siRNAs having modification of the sugar group of a nucleotide, modification of a nucleobase of a nucleotide, modification of a nucleic acid backbone or linkage, modification of the structure of a nucleotide or nucleotides at the terminus of an siRNA strand, and combinations thereof.

Examples of siRNAs with modified nucleotides include siRNAs having modification of the substituent at the 2′ carbon of the sugar. Examples of siRNAs with modified nucleotides include siRNAs having modification at the 5′ end, the 3′ end, or at both ends of a strand. Examples of modified siRNAs include siRNAs having modifications that produce complementarity mismatches between the strands.

Examples of siRNAs with modified nucleotides include siRNAs having a 5′-propylamine end, a 5′-phosphorylated end, a 3′-puromycin end, or a 3′-biotin end group.

Examples of siRNAs with modified nucleotides include siRNAs having a 2′-fluoro substituted ribonucleotide, a 2′-OMe substituted ribonucleotide, a 2′-deoxy ribonucleotide, a 2′-amino substituted ribonucleotide, or a 2′-thio substituted ribonucleotide.

Examples of siRNAs with modified nucleotides include siRNAs having one or more 5-halouridines, 5-halocytidines, 5-methylcytidines, ribothymidines, 2-aminopurines, 2,6-diaminopurines, 4-thiouridines, or 5-aminoallyluridines.

Examples of siRNAs with modified nucleotides include siRNAs having one or more phosphorothioate groups.

Examples of siRNAs with modified nucleotides include siRNAs having one or more 2′-fluoro substituted ribonucleotides, 2′-fluorouridines, 2′-fluorocytidines, 2′-deoxyribonucleotides, 2′-deoxyadenosines, or 2′-deoxyguanosines.

Examples of siRNAs with modified nucleotides include siRNAs having one or more phosphorothioate linkages.

Examples of siRNAs with modified nucleotides include siRNAs having one or more alkylene diol linkages, oxy-alkylthio linkages, or oxycarbonyloxy linkages.

Examples of siRNAs with modified nucleotides include siRNAs having one or more deoxyabasic groups, inosines, N3-methyl-uridines, N6,N6-dimethyl-adenosines, pseudouridines, purine ribonucleosides, or ribavirins.

Examples of siRNAs with modified nucleotides include siRNAs having one or more 3′ or 5′ inverted terminal groups.

Examples of siRNAs with modified nucleotides include siRNAs having one or more 5-(2-amino)propyluridines, 5-bromouridines, adenosines, 8-bromo guanosines, 7-deaza-adenosines, or N6-methyl adenosine.

In some embodiments, the number of modified nucleotides of the nucleic acid, siRNA molecule, sense strand, or antisense strand as described herein comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, nucleotides, or an amount of nucleotides within a range defined by any two of the aforementioned numbers of nucleotides (depending on the length of the nucleic acid, siRNA molecule, sense strand, or antisense strand).

In some embodiments, the nucleic acid, siRNA molecule, sense strand, or antisense strand as described herein comprises or consists of modified nucleotides at any one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 of the nucleic acid, siRNA molecule, sense strand, or antisense strand (depending on the length of the nucleic acid, siRNA molecule, sense strand, or antisense strand). In some embodiments, the nucleic acid, siRNA molecule, sense strand, or antisense strand as described herein comprises or consists of modified nucleotides at each of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 of the nucleic acid, siRNA molecule, sense strand, or antisense strand (depending on the length of the nucleic acid, siRNA molecule, sense strand, or antisense strand).

### RNA Interference

In some embodiments, RNA interference (RNAi) refers to sequence-specific post-transcriptional gene silencing in animals mediated by short interfering RNAs (siRNAs). An RNAi response in cells can be triggered by a double stranded RNA (dsRNA). Certain dsRNAs in cells can undergo the action of Dicer enzyme, or a ribonuclease III enzyme. Dicer can process the dsRNA into shorter pieces of dsRNA, which are siRNAs. In general, siRNAs can be from about 21 to about 23 nucleotides in length and include a base pair duplex region about 19 nucleotides in length.

RNAi molecules or siRNAs can down regulate or knock down gene expression by mediating RNA interference in a sequence-specific manner. RNAi molecules or siRNAs can also be used to knock down viral gene expression, and therefore affect viral replication. RNAi typically involves an endonuclease complex known as the RNA induced silencing complex (RISC). An siRNA may have an antisense or guide strand which enters the RISC complex and mediates cleavage of a single stranded RNA target having a sequence complementary to the antisense strand of the siRNA duplex. In some embodiments, the siRNA has another strand of the siRNA, that is a passenger strand. Cleavage of the target RNA may take place in the middle of the region complementary to the antisense strand of the siRNA duplex.

As used herein, the term “sense strand” refers to a nucleotide sequence of an siRNA molecule that is partially or fully complementary to at least a portion of a corresponding antisense strand of the siRNA molecule. The sense strand of an siRNA molecule can include a nucleic acid sequence having homology with a target nucleic acid sequence.

As used herein, the term “antisense strand” refers to a nucleotide sequence of an siRNA molecule that is partially or fully complementary to at least a portion of a target nucleic acid sequence. The antisense strand of an siRNA molecule can include a nucleic acid sequence that is complementary to at least a portion of a corresponding sense strand of the siRNA molecule.

As used herein, the terms “inhibit,” “down-regulate,” or “reduce” with respect to gene expression means that the expression of the gene, or the level of mRNA molecules encoding one or more proteins, or the activity of one or more of the encoded proteins is reduced below that observed in the absence of an RNAi molecule or siRNA of this disclosure. For example, the level of expression, level of mRNA, or level of encoded protein activity may be reduced by at least 1%, or at least 10%, or at least 20%, or at least 50%, or at least 90%, or more from that observed in the absence of an RNAi molecule or siRNA of this disclosure.

RNAi molecules or siRNAs can be made from separate polynucleotide strands: a sense strand or passenger strand, and an antisense strand or guide strand. The guide and passenger strands are at least partially complementary. The guide strand and passenger strand can form a duplex region having from or about from 15 to 49 base pairs or about 49 base pairs.

In some embodiments, the duplex region of an siRNA can have 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs, or a range of base pairs defined by any two of the aforementioned numbers of base pairs.

In certain embodiments, an RNAi molecule or siRNA can be active in a RISC complex, with a length of duplex region active for RISC. In additional embodiments, an RNAi molecule can be active as a Dicer substrate, to be converted to an RNAi molecule that can be active in a RISC complex.

In some aspects, an RNAi molecule or siRNA can have complementary guide and passenger sequence portions at opposing ends of a long molecule, so that the molecule can form a duplex region with the complementary sequence portions, and the strands are linked at one end of the duplex region by either nucleotide or non-nucleotide linkers. For example, a hairpin arrangement, or a stem and loop arrangement. The linker interactions with the strands can be covalent bonds or non-covalent interactions.

A RNAi molecule or siRNA of this disclosure may include a nucleotide, non-nucleotide, or mixed nucleotide/non-nucleotide linker that joins the sense region of the nucleic acid to the antisense region of the nucleic acid. A nucleotide linker can be a linker of 2 or more nucleotides in length, for example about 3, 4, 5, 6, 7, 8, 9, or 10 nucleotides in length. The nucleotide linker can be a nucleic acid aptamer. By “aptamer” or “nucleic acid aptamer” as used herein refers to a nucleic acid molecule that binds specifically to a target molecule wherein the nucleic acid molecule has sequence that includes a sequence recognized by the target molecule in its natural setting. Alternately, an aptamer can be a nucleic acid molecule that binds to a target molecule, where the target molecule does not naturally bind to a nucleic acid. For example, the aptamer can be used to bind to a ligand-binding domain of a protein, thereby preventing interaction of the naturally occurring ligand with the protein.

Examples of non-nucleotide linkers include an abasic nucleotide, polyether, polyamine, polyamide, peptide, carbohydrate, lipid, polyhydrocarbon, or other polymeric compounds, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc., 1991, Vol. 113, pp. 6324-6326; Jaeschke et al., Tetrahedron Lett., 1993, Vol. 34, pp. 301; Arnold et al., WO1989/002439; Usman et al., WO1995/006731; Dudycz et al., WO1995/011910, and Ferentz et al., J. Am. Chem. Soc., 1991, Vol. 113, pp. 4000-4002, all of which are hereby expressly incorporated by reference in their entireties.

A RNAi molecule or siRNA can have one or more overhangs from the duplex region. The overhangs, which are non-base-paired, single strand regions, can be from one to five nucleotides in length, or longer. An overhang can be a 3′-end overhang, wherein the 3′-end of a strand has a single strand region of from one to eight nucleotides. An overhang can be a 5′-end overhang, wherein the 5′-end of a strand has a single strand region of from one to eight nucleotides. The overhangs of an RNAi molecule or siRNA can have the same length, or different lengths.

A RNAi molecule or siRNA can have one or more blunt ends, in which the duplex region ends with no overhang, and the strands are base paired to the end of the duplex region. A RNAi molecule or siRNA of this disclosure can have one or more blunt ends, or can have one or more overhangs, or can have a combination of a blunt end and an overhang end. A 5′-end of a strand of an RNAi molecule or siRNA may be in a blunt end or can be in an overhang. A 3′-end of a strand of an RNAi molecule or siRNA may be in a blunt end or can be in an overhang. A 5′-end of a strand of an RNAi molecule or siRNA may be in a blunt end, while the 3′-end is in an overhang. A 3′-end of a strand of an RNAi molecule or siRNA may be in a blunt end, while the 5′-end is in an overhang. In some embodiments, both ends of an RNAi molecule or siRNA are blunt ends. In additional embodiments, both ends of an RNAi molecule have an overhang. The overhangs at the 5′- and 3′-ends may be of different lengths.

In certain embodiments, an RNAi molecule or siRNA may have a blunt end where the 5′-end of the antisense strand and the 3′-end of the sense strand do not have any overhanging nucleotides. In further embodiments, an RNAi molecule or siRNA may have a blunt end where the 3′-end of the antisense strand and the 5′-end of the sense strand do not have any overhanging nucleotides. A RNAi molecule or siRNA may have mismatches in base pairing in the duplex region. Any nucleotide in an overhang of an RNAi molecule or siRNA can be a deoxyribonucleotide, or a ribonucleotide. One or more deoxyribonucleotides may be at the 5′-end, where the 3′-end of the other strand of the RNAi molecule or siRNA may not have an overhang, or may not have a deoxyribonucleotide overhang. One or more deoxyribonucleotides may be at the 3′-end, where the 5′-end of the other strand of the RNAi molecule or siRNA may not have an overhang, or may not have a deoxyribonucleotide overhang. In some embodiments, one or more, or all of the overhang nucleotides of an RNAi molecule or siRNA may be 2′-deoxyribonucleotides.

### Dicer Substrates

In some aspects, an siRNA or RNAi molecule can be of a length suitable as a Dicer substrate, which can be processed to produce a RISC active RNAi molecule (see, e.g., Rossi et al., US2005/0244858, which is hereby expressly incorporated by reference in its entirety).

A double stranded RNA (dsRNA) that is a Dicer substrate can be of a length sufficient such that it is processed by Dicer to produce an active RNAi molecule, and may further include one or more of the following properties: (i) the Dicer substrate dsRNA can be asymmetric, for example, having a 3′ overhang on the antisense strand, or (ii) the Dicer substrate dsRNA can have a modified 3′ end on the sense strand to direct orientation of Dicer binding and processing of the dsRNA to an active RNAi molecule.

In certain embodiments, the longest strand in a Dicer substrate dsRNA may be 24-30 nucleotides in length. A Dicer substrate dsRNA can be symmetric or asymmetric. In some embodiments, a Dicer substrate dsRNA can have a sense strand of 22-28 nucleotides and an antisense strand of 24-30 nucleotides. In certain embodiments, a Dicer substrate dsRNA may have an overhang on the 3′ end of the antisense strand. In further embodiments, a Dicer substrate dsRNA may have a sense strand 25 nucleotides in length, and an antisense strand 27 nucleotides in length, with a 2 base 3′-overhang. The overhang may be 1, 2 or 3 nucleotides in length. The sense strand may also have a 5′ phosphate. An asymmetric Dicer substrate dsRNA may have two deoxyribonucleotides at the 3′-end of the sense strand in place of two of the ribonucleotides.

The sense strand of a Dicer substrate dsRNA may be from or from about 22 to 30 or about 30, or from or from about 22 to 28 or about 28; or from or from about 24 to 30 or about 30; or from or from about 25 to 30 or about 30; or from or from about 26 to 30 or about 30; or from or from about 26 and 29 or about 29; or from or from about 27 to 28 or about 28 nucleotides in length. The sense strand of a Dicer substrate dsRNA may be 22, 23, 24, 25, 26, 27, 28, 29 or 30 nucleotides in length. In certain embodiments, a Dicer substrate dsRNA may have sense and antisense strands that are at least or at least about 25 nucleotides in length, and no longer than 30 or about 30 nucleotides in length. In certain embodiments, a Dicer substrate dsRNA may have sense and antisense strands that are 26 to 29 nucleotides in length. In certain embodiments, a Dicer substrate dsRNA may have sense and antisense strands that are 27 nucleotides in length. The sense and antisense strands of a Dicer substrate dsRNA may be the same length as in being blunt ended, or different lengths as in having overhangs, or may have a blunt end and an overhang. A Dicer substrate dsRNA may have a duplex region of 19, 20, 21, 22, 23, 24, 25, 26 or 27 nucleotides in length.

The antisense strand of a Dicer substrate dsRNA may have any sequence that anneals to at least a portion of the sequence of the sense strand under biological conditions, such as within the cytoplasm of a eukaryotic cell. A Dicer substrate with a sense and an antisense strand can be linked by a third structure, such as a linker group or a linker oligonucleotide. The linker connects the two strands of the dsRNA, for example, so that a hairpin is formed upon annealing. The sense and antisense strands of a Dicer substrate are in general complementary but may have mismatches in base pairing.

In some embodiments, a Dicer substrate dsRNA can be asymmetric such that the sense strand has 22-28 nucleotides and the antisense strand has 24-30 nucleotides. A region of one of the strands, particularly the antisense strand, of the Dicer substrate dsRNA may have a sequence length of at least 19 nucleotides, wherein these nucleotides are in the 21-nucleotide region adjacent to the 3′ end of the antisense strand and are sufficiently complementary to a nucleotide sequence of the RNA produced from the target gene. An antisense strand of a Dicer substrate dsRNA can have from 1 to 9 ribonucleotides on the 5′-end, to give a length of 22-28 nucleotides. When the antisense strand has a length of 21 nucleotides, then 1-7 ribonucleotides, or 2-5 ribonucleotides, or 4 ribonucleotides may be added on the 3′-end. The added ribonucleotides may have any sequence. A sense strand of a Dicer substrate dsRNA may have 24-30 nucleotides. The sense strand may be substantially complementary with the antisense strand to anneal to the antisense strand under biological conditions.

Methods for Using siRNAs

The nucleic acid molecules, siRNAs and RNAi molecules of this disclosure may be delivered to a cell or tissue by direct application of the molecules, or with the molecules combined with a carrier or a diluent. The nucleic acid molecules, siRNAs and RNAi molecules of this disclosure can be delivered or administered to a cell, tissue, organ, or subject by direct application of the molecules with a carrier or diluent, or any other delivery vehicle that acts to assist, promote or facilitate entry into a cell, for example, viral sequences, viral material, or lipid or liposome formulations.

The nucleic acid molecules, siRNAs and RNAi molecules of this disclosure can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection.

Delivery systems may include, for example, aqueous and nonaqueous gels, creams, emulsions, microemulsions, liposomes, ointments, aqueous and nonaqueous solutions, lotions, aerosols, hydrocarbon bases or powders, and can contain excipients such as solubilizers or permeation enhancers.

Compositions and methods of this disclosure can include an expression vector that includes a nucleic acid sequence encoding at least one siRNA RNAi molecule of this disclosure in a manner that allows expression of the nucleic acid molecule.

The nucleic acid molecules, siRNAs and RNAi molecules of this disclosure can be expressed from transcription units inserted into DNA or RNA vectors. Recombinant vectors can be DNA plasmids or viral vectors. Viral vectors can be used that provide for transient expression of nucleic acid molecules. For example, the vector may contain sequences encoding both strands of an RNAi molecule or siRNA of a duplex, or a single nucleic acid molecule that is self-complementary and thus forms an RNAi molecule or siRNA. An expression vector may include a nucleic acid sequence encoding two or more nucleic acid molecules.

A nucleic acid molecule may be expressed within cells from eukaryotic promoters. Those skilled in the art realize that any nucleic acid can be expressed in eukaryotic cells from the appropriate DNA/RNA vector. In some aspects, a viral construct can be used to introduce an expression construct into a cell, for transcription of a dsRNA construct encoded by the expression construct. Lipid formulations can be administered to animals by intravenous, intramuscular, or intraperitoneal injection, or orally or by inhalation or other methods as are known in the art. Pharmaceutically acceptable formulations for administering oligonucleotides are known and can be used.

### Formulations for Delivery of Active Agent to Lung

Various embodiments of this disclosure provide compounds and compositions for use in therapeutic formulations for delivery to the lung. In some aspects, this disclosure relates to compounds, compositions and methods for providing nanoparticles to deliver and distribute an active pharmaceutical ingredient (API) to the lung in amounts effective for inhibiting expression of a Coronavirus protein. Examples of suitable formulations that provide delivery of APIs to the lung include those described in International Application No. PCT/US2019/061702 and U.S. Patent Publication No. 2020/0157540 (U.S. application Ser. No. 16/685,283), both of which were filed on Nov. 15, 2019. Both of the aforementioned applications are hereby incorporated herein by reference in their entireties for all purposes, and particularly for describing suitable formulations capable of providing delivery of APIs to the lung, including such delivery of the active nucleic acid APIs (such as an active dsRNA) as described herein.

Various embodiments of this disclosure provide a formulation designed for clinical use containing an active nucleic acid API (such as an active dsRNA) as described herein that can decrease expression of a Coronavirus protein.

Embodiments of a pharmaceutical composition of this disclosure may contain an ionizable lipid of the Formula I or II (such as Compound A) for delivering the active nucleic acid API (e.g., dsRNA) to cells in the lung. The ionizable lipid of the Formula I or II (e.g., Compound A), along with additional lipid components in a formulation of this disclosure can be used to form nanoparticles to deliver and distribute the active nucleic acid API (e.g., siRNA) to the lung for treating or ameliorating MERS, SARS and/or Covid-19.

Embodiments of a pharmaceutical composition of this disclosure can contain a 1,2-distearoyl-sn-glycero-3-phosphoethanolamine (DSPE) lipid compound for delivering the active nucleic acid API (e.g., siRNA) to one or more types of lung cells. The DSPE lipid compound, which can be DSPE-mPEG-2000, along with additional lipid components in a formulation of this disclosure can be used to form nanoparticles to deliver and distribute the active nucleic acid API (e.g. siRNA) for treating or ameliorating MERS, SARS and/or Covid-19.

In some embodiments, a pharmaceutical composition of this disclosure containing an active agent that is a dsRNA targeted to Coronavirus, along with an ionizable lipid of the Formula I or II (e.g., Compound A) and a DSPE-mPEG-2000 lipid component, can exhibit enhanced distribution of the active agent to lung by parenteral administration.

Embodiments of this disclosure include a pharmaceutical solution of a pharmaceutical composition, which is suitable for lyophilization. A pharmaceutical solution of a pharmaceutical composition may contain solvents, for example, ethanol and water for injection, as well as protective agents, for example, sucrose and 2-hydroxypropyl-β-cyclodextrin, which protect the active agent during lyophilization. A pharmaceutical solution can also contain a buffer. A pharmaceutical solution allows a suspension of a pharmaceutical composition to be made, which can be maintained during lyophilization.

In further embodiments, this disclosure includes a solid lyophile composition made from a pharmaceutical solution. The pharmaceutical solution can be lyophilized to a solid cake or powder, which maintains the activity of the active agent of the pharmaceutical solution.

Various embodiments of a solid lyophile composition of this disclosure can be prepared in a vial or other container for use as a drug product. A kit utilizing the vial of the solid lyophile composition can contain instructions for using the drug product, and for preparing a reconstituted drug from the solid lyophile composition.

In further embodiments, an active agent of this disclosure can be delivered using a reconstituted form of a solid lyophile composition. The reconstituted solution form of a solid lyophile composition, which may be prepared with a sterile diluent, can be used as a drug for parenteral delivery.

In another aspect, embodiments of this disclosure provide methods for utilizing therapeutic compositions that decrease the expression of a Coronavirus gene or protein. The therapeutic compositions of embodiments of this disclosure can include an inhibitory nucleic acid molecule, such as a siRNA or shRNA.

Various embodiments provide formulations with four or more lipid components for delivery of active agents to the lung. This disclosure can provide a composition for use in distributing an active agent in cells, tissues or organs, organisms, and subjects, where the composition includes one or more ionizable lipid molecules of this disclosure. Compositions of embodiments of this disclosure may include one or more of the ionizable lipid molecules, along with a structural lipid, one or more stabilizer lipids, and one or more lipids for reducing immunogenicity of the nanoparticles.

An embodiment provides a pharmaceutical composition, comprising:
(a) an effective amount of a dsRNA as described herein;
(b) an ionizable lipid compound having the following Formula II:

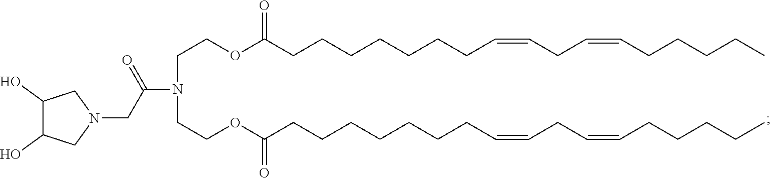 [SMILES: CCCCC/C=C\C/C=C\CCCCCCCC(=O)OCCN(CCOC(=O)CCCCCCC/C=C\C/C=C\CCCCC)C(=O)CN1CC(O)C(O)C1]

(c) a DSPE lipid comprising a polyethyleneglycol (PEG) region, a multi-branched PEG region, a methoxypolyethyleneglycol (mPEG) region, a carbonyl-methoxypolyethyleneglycol region, or a polyglycerine region;
(d) a sterol lipid; and
(e) one or more neutral lipids.

Various ionizable lipids are described elsewhere herein. An ionizable lipid molecule of embodiments of this disclosure can be any mol % of a composition of this disclosure. The ionizable lipid molecules of a composition of embodiments of this disclosure can be from 15 mol % to 35 mol % of the lipid components of the composition. In certain embodiments, the ionizable lipid molecules of a composition can be from 15 mol % to 25 mol %, or from 20 mol % to 30 mol % of the lipid components of the composition.

Various structural lipids are described elsewhere herein. The structural lipid of a composition of embodiments of this disclosure can be from 25 mol % to 40 mol % of the lipid components of the composition. In certain embodiments, the structural lipid of a composition can be from 25 mol % to 35 mol %, or from 30 mol % to 40 mol % of the lipid components of the composition.

Various stabilizer lipids are described elsewhere herein. The sum of the stabilizer lipids of a composition of embodiments of this disclosure can be from 25 mol % to 45% mol % of the lipid components of the composition. In certain embodiments, the sum of the stabilizer lipids of a composition can be from 30 mol % to 40 mol % of the lipid components of the composition.

In certain embodiments, the sum of the one or more stabilizer lipids can be from 25 mol % to 45 mol % of the lipids of the composition, wherein each of the stabilizer lipids individually can be from 5 mol % to 40% mol %.

In certain embodiments, the sum of the one or more stabilizer lipids can be from 30 mol % to 40 mol % of the lipids of the composition, wherein each of the stabilizer lipids individually can be from 10 mol % to 30% mol %. In various embodiments, the structural lipids (e.g., cholesterol) and the stabilizer lipids (e.g., DOPC and DOPE) combined comprise 50 mol % to 85 mol % of the total lipids of the composition.

The one or more lipids for reducing immunogenicity of the nanoparticles can be from a total of 1 mol % to 10 mol %, or 1 mol % to 8 mol % of the lipid components of the composition. In certain embodiments, the one or more lipids for reducing immunogenicity of the nanoparticles can be from a total of 1 mol % to 5 mol % of the lipid components of the composition.

In compositions of this disclosure, the entirety of the lipid components may include one or more of the ionizable lipid molecular components, one or more structural lipids, one or more stabilizer lipids, and one or more lipids for reducing immunogenicity of the nanoparticles.

Examples of formulations of embodiments of this disclosure are shown in Table 6.

TABLE 6
Examples of pharmaceutical compositions

| | | | | | | | | | Zeta at |
|---|---|---|---|---|---|---|---|---|---|
| | Cmpd A | Cholesterol | DOPC | DOPE | DSPE-mPEG | N/P | Z-Ave | | pH 5.5 |
| No. | % | % | % | % | % | Ratio | (nm) | PDI | (mV) |
| 1 | 23.5 | 30 | 20 | 20 | 6.5 | 2.5 | 37 | 0.08 | −1.5 |
| 2 | 10 | 40 | 10 | 30 | 10 | 4 | 31 | 0.18 | −8.3 |
| 3 | 25 | 30 | 20 | 20 | 5 | 1.5 | 46 | 0.07 | −0.1 |
| 4 | 10 | 40 | 10 | 30 | 10 | 1 | 35 | 0.28 | −5.6 |
| 5 | 10 | 40 | 30 | 10 | 10 | 1 | 35 | 0.17 | −6 |

Examples of an ionizable lipid include compounds having the structure shown in Formula I:

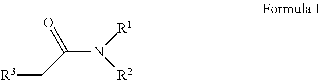

wherein R¹ and R² are

R¹═CH₂(CH₂)ₙOC(═O)R⁴

R²═CH₂(CH₂)ₘOC(═O)R⁵

wherein n and m are from 1 to 2; and R⁴ and R⁵ are independently for each occurrence a C(12-20) alkyl group, or a C(12-20) alkenyl group having from zero to two double bonds;

wherein R³ is selected from

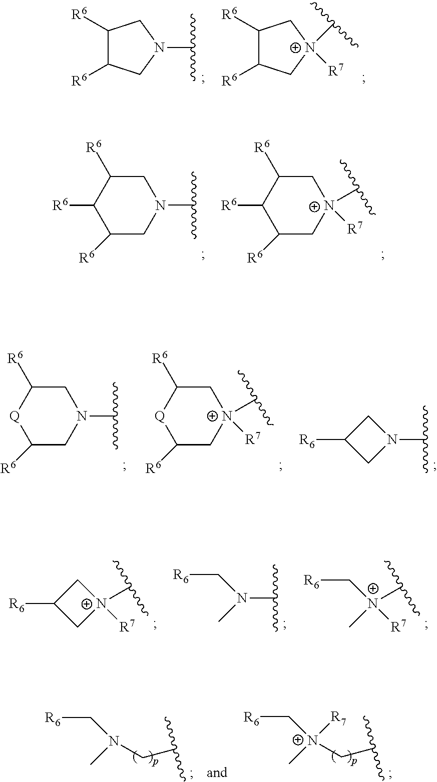

wherein:

R⁶ is selected from H, alkyl, hydroxy, hydroxyalkyl, alkoxy, alkoxyalkoxy, aminoalkyl;

R⁷ is selected from H, alkyl, hydroxyalkyl;

Q is O or NR⁷;

p is from 1 to 4.

Examples of ionizable lipids include compounds of the following Formula II:

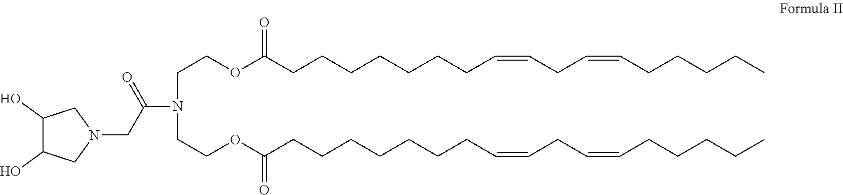 [SMILES: CCCCC/C=C\C/C=C\CCCCCCCC(=O)OCCN(CCOC(=O)CCCCCCC/C=C\C/C=C\CCCCC)C(=O)CN1CC(O)C(O)C1]

The following Compound A, which is ((2-((3S,4R)-3,4-dihydroxypyrrolidin-1-yl)acetyl)azanediyl)bis(ethane-2,1-diyl) (9Z,9′Z,12Z,12′Z)-bis(octadeca-9,12-dienoate), is an example of an ionizable lipid of the Formula II:

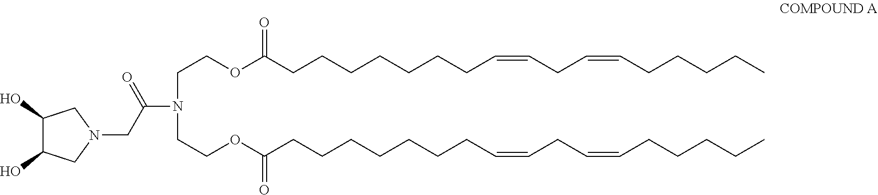 [SMILES: CCCCC/C=C\C/C=C\CCCCCCCC(=O)OCCN(CCOC(=O)CCCCCCC/C=C\C/C=C\CCCCC)C(=O)CN1C[C@@H](O)[C@@H](O)C1]

Examples of structural lipids include cholesterols, sterols, and steroids.

Examples of structural lipids include cholanes, cholestanes, ergostanes, campestanes, poriferastanes, stigmastanes, gorgostanes, lanostanes, gonanes, estranes, androstanes, pregnanes, and cycloartanes.

Examples of structural lipids include sterols and zoosterols such as cholesterol, lanosterol, zymosterol, zymostenol, desmosterol, stigmastanol, dihydrolanosterol, and 7-dehydrocholesterol.

Examples of structural lipids include pegylated cholesterols, and cholestane 3-oxo-(C1-22)acyl compounds, for example, cholesteryl acetate, cholesteryl arachidonate, cholesteryl butyrate, cholesteryl hexanoate, cholesteryl myristate, cholesteryl palmitate, cholesteryl behenate, cholesteryl stearate, cholesteryl caprylate, cholesteryl n-decanoate, cholesteryl dodecanoate, cholesteryl nervonate, cholesteryl pelargonate, cholesteryl n-valerate, cholesteryl oleate, cholesteryl elaidate, cholesteryl erucate, cholesteryl heptanoate, cholesteryl linolelaidate, and cholesteryl linoleate.

Examples of structural lipids include sterols such as phytosterols, beta-sitosterol, campesterol, ergosterol, brassicasterol, delta-7-stigmasterol, and delta-7-avenasterol.

Examples of stabilizer lipids include zwitterionic lipids. Examples of stabilizer lipids include compounds such as phospholipids. Examples of phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine and ordilinoleoylphosphatidylcholine.

Examples of stabilizer lipids include phosphatidyl ethanolamine compounds and phosphatidyl choline compounds. Examples of stabilizer lipids include 1,2-Dioleoyl-sn-Glycero-3-Phosphocholine (DOPC). Examples of stabilizer lipids include diphytanoyl phosphatidyl ethanolamine (DPhPE) and 1,2-Diphytanoyl-sn-Glycero-3-Phosphocholine (DPhPC). Examples of stabilizer lipids include 1,2-distearoyl-sn-glycero-3-phosphocholine (DSPC), 1,2-dipalmitoyl-sn-glycero-3-phosphocholine (DPPC), 1,2-dipalmitoyl-sn-glycero-3-phosphoethanolamine (DPPE), and 1,2-dioleoyl-sn-glycero-3-phosphoethanolamine (DOPE).

Examples of stabilizer lipids include 1,2-dilauroyl-sn-glycerol (DLG); 1,2-dimyristoyl-sn-glycerol (DMG); 1,2-dipalmitoyl-sn-glycerol (DPG); 1,2-distearoyl-sn-glycerol (DS G); 1,2-diarachidoyl-sn-glycero-3-phosphocholine (DAPC); 1,2-dilauroyl-sn-glycero-3-phosphocholine (DLPC); 1,2-dimyristoyl-sn-glycero-3-phosphocholine (DMPC); 1,2-dipalmitoyl-sn-glycero-O-ethyl-3-phosphocholine (DPePC); 1,2-dilauroyl-sn-glycero-3-phosphoethanolamine (DLPE); 1,2-dimyristoyl-sn-glycero-3-phosphoethanolamine (DMPE); 1,2-distearoyl-sn-glycero-3-phosphoethanolamine (DSPE); 1-palmitoyl-2-linoleoyl-sn-glycero-3-phosphocholine; 1-palmitoyl-2-oleoyl-sn-glycero-3-phosphocholine (POPC); 1-palmitoyl-2-lyso-sn-glycero-3-phosphocholine (P-Lyso-PC); and 1-Stearoyl-2-lyso-sn-glycero-3-phosphocholine (S-Lyso-PC).

As used herein, the term DMPE-mPEG-2000 refers to 1,2-distearoyl-sn-glycero-3-phosphoethanolamine-N-[methoxy(polyethylene glycol)-2000]. Examples of lipids for reducing immunogenicity include polymeric compounds and polymer-lipid conjugates.

Examples of lipids for reducing immunogenicity include pegylated lipids having polyethyleneglycol (PEG) regions. The PEG regions can be of any molecular mass. In some embodiments, a PEG region can have an average molecular mass of 200, 300, 350, 400, 500, 550, 750, 1000, 1500, 2000, 3000, 3500, 4000 or 5000 Da.

Examples of lipids for reducing immunogenicity include compounds having a methoxypolyethyleneglycol region. Examples of lipids for reducing immunogenicity include compounds having a carbonyl-methoxypolyethyleneglycol region.

Examples of lipids for reducing immunogenicity include compounds having a multi-branched PEG region. Examples of lipids for reducing immunogenicity include compounds having a polyglycerine region. Examples of lipids for reducing immunogenicity include polymeric lipids such as DSPE-mPEG, DMPE-mPEG, DPPE-mPEG, and DOPE-mPEG.

Examples of lipids for reducing immunogenicity include PEG-phospholipids and PEG-ceramides.

The lipids described herein can be combined in various ways to form lipid compositions. For example, in some embodiments, a composition can contain the ionizable lipid of the Formula II (e.g., Compound A), the structural lipid cholesterol, the stabilizer lipids DOPC and DOPE, and the lipid for reducing immunogenicity DSPE-mPEG. In certain embodiments, the ionizable lipid of the Formula II (e.g., Compound A) can comprise 15 mol % to 35 mol %, or 15 mol % to 25 mol %, or 20 mol % to 30 mol % of the composition; the cholesterol can comprise from 25 mol % to 35 mol % of the total lipids of the composition; the DOPC and DOPE combined can comprise from 30 mol % to 50 mol % of the total lipids of the composition; the cholesterol, DOPC, and DOPE combined can comprise 50 mol % to 85 mol %, or 60 mol % to 80 mol %, or 75 mol % to 85 mol % of the composition; and DSPE-mPEG can comprise from 1 mol % to 8 mol %, or from 4 mol % to 6 mol %, or 5 mol %, of the total lipids of the composition. In various embodiments, the compound of Formula II, cholesterol, DOPC, DOPE, and DSPE-mPEG-2000 combined comprise at least 97 mol % of the total lipids of the composition. For example, in an embodiment the compound of Formula II, cholesterol, DOPC, DOPE, and DSPE-mPEG-2000 combined comprise about 100 mol % of the total lipids of the composition.

In one embodiment, the ionizable lipid of the Formula II (e.g., Compound A) can be 25 mol % of the total lipids of the composition; cholesterol can be 30 mol % of the total lipids of the composition, DOPC can be 20 mol % of the total lipids of the composition, DOPE can be 20 mol % of the total lipids of the composition; and DSPE-mPEG(2000) can be 5 mol % of the total lipids of the composition.

Embodiments of this disclosure can provide liposome nanoparticle compositions. The ionizable molecules of embodiments of this disclosure can be used to form liposome compositions, which can have a bilayer of lipid-like molecules. A nanoparticle composition can have one or more of the ionizable molecules of this disclosure in a liposomal structure, a bilayer structure, a micelle, a lamellar structure, or a mixture thereof.

In some embodiments, a composition can include one or more liquid vehicle components. A liquid vehicle suitable for delivery of active agents of this disclosure can be a pharmaceutically acceptable liquid vehicle. A liquid vehicle can include an organic solvent, or a combination of water and an organic solvent.

Embodiments of this disclosure can provide lipid nanoparticles having a size of from 10 nm to 1000 nm. In some embodiments, the liposome nanoparticles can have a size of from 10 nm to 150 nm.

In certain embodiments, the liposome nanoparticles of this disclosure can encapsulate the RNAi molecule and retain at least 80% of the encapsulated RNAi molecules after 1 hour exposure to human serum. As used herein, the term “encapsulate” refers to the ability of a nanoparticle to carry an active agent within its structure, or on its surface, such that the active agent is not removed by solvent or mobile phase exterior to the particle.

### Lyophilized Nanoparticle Formulations

In some aspects, delivery of an active agent to the lung in vivo can surprisingly be accomplished with a nanoparticle formulation of this disclosure that can be lyophilized, reconstituted, and injected intravenously. Embodiments of this disclosure further provide lyophile forms of nanoparticles that can be reconstituted into effective therapeutic compositions, which can be used to deliver therapeutic nucleic acid agents for transfection.

In some aspects, this disclosure provides compositions and compounds for forming solutions or suspensions of therapeutic lipid nanoparticles that are stable in lyophilization processes. The therapeutic lipid nanoparticles can encapsulate nucleic acid agents, and can be transformed and stored in solid lyophile forms. The lyophile forms can be reconstituted to provide therapeutic lipid nanoparticles with encapsulated nucleic acid agents. The reconstituted lipid nanoparticles can have surprisingly advantageous transfection properties, including particle size and distribution. Embodiments of this disclosure include a range of compositions and compounds for forming solutions or suspensions of therapeutic lipid nanoparticles that can undergo a lyophilization process to provide stable, solid lyophile forms for long-term storage of a nucleic acid therapeutic.

In further aspects, this disclosure provides compounds and methods for forming solutions or suspensions of therapeutic lipid nanoparticles that are stable in lyophilization processes. The lyophilization processes of this disclosure can provide stable lyophile forms of therapeutic lipid nanoparticles, in which the nanoparticles can encapsulate nucleic acid agents. The lyophile forms can be stored for a period of time, and reconstituted to provide therapeutic lipid nanoparticles with encapsulated nucleic acid agents.

In some embodiments, this disclosure includes a range of compositions and compounds for solutions or suspensions of lipid nanoparticles that can undergo a lyophilization process to provide stable, solid lyophile forms for long-term storage of a nucleic acid therapeutic. Compositions and processes of this disclosure can provide lyophile forms that can be reconstituted and provide advantageous activity, particle size, storage time, and serum stability.

In further aspects, this disclosure relates to compounds, compositions and methods for providing nanoparticles to deliver and distribute active agents or drug compounds to the lung. In an embodiment, this disclosure provides a range of lipid compounds and ionizable compounds for delivering active agents to lung cells. The lipid compounds and ionizable compounds of this disclosure can be used to form nanoparticles to deliver and distribute active agents.

In an embodiment, this disclosure contemplates lipid nanoparticle drug formulations containing, for example, siRNA agents, which can be prepared by lyophilization of a suspension of the nanoparticles, and reconstitution of the nanoparticles into a suspension.

In some embodiments, lipid nanoparticles can be synthesized by high speed injection of lipid/ethanol solution into an siRNA buffer solution. A second buffer can be diafiltered and used as an external buffer through TFF cartridges to make a final product aqueous suspension.

In some embodiments, the nanoparticles can have an average diameter of from 45 nm to 110 nm. The concentration of the nucleic acid active agents can be from 1 mg/mL to 10 mg/mL. It was found that lipid nanoparticles can survive lyophilization of the suspension, when the suspension is made into a protected composition.

In some embodiments, a protected composition of this disclosure can be composed of an aqueous suspension of the lipid nanoparticles in a pharmaceutically acceptable solution, a dextrin compound, and a saccharide sugar compound. The lipid nanoparticles can encapsulate an active agent, such as one or more nucleic acid active agents. Lyophilization of the protected suspension can provide a solid lyophile product, which can be reconstituted into a suspension of lipid nanoparticles. The reconstituted suspension can contain lipid nanoparticles, which encapsulate the active agent and are comparable to the lipid nanoparticles before lyophilization. In certain embodiments, the reconstituted suspension can provide activity of the encapsulated agent, which is comparable to that of the suspension before lyophilization. In further aspects, the reconstituted suspension can provide stable nanoparticles comparable to that of the suspension before lyophilization. In certain aspects, the average particle size of the nanoparticles can be nearly equal to the size of the nanoparticles in the suspension before lyophilization.

In an embodiment, the compositions and processes of this disclosure can provide surprising activity and stability of a reconstituted suspension composed of nanoparticles having an encapsulated agent.

In further aspects, the protected suspension, which can be lyophilized and reconstituted, can contain a protectant composition for lyophilization. A protectant composition of this disclosure can be composed of a dextrin compound and a saccharide sugar compound. The total amount of the dextrin and sugar compounds may be from 2% to 20% (w/v) of the protected suspension.

In some embodiments, the dextrin compound can be from 40% to 70% (w/v) of the total amount of the dextrin and sugar compounds in the protectant composition. In certain embodiments, the dextrin compound can be from 40% to 55% (w/v) of the total amount of the dextrin and sugar compounds in the protectant composition. In further embodiments, the dextrin compound may be from 40% to 45% (w/v) of the total amount of the dextrin and sugar compounds in the protectant composition. These compositions can provide unexpectedly advantageous properties of a reconstituted nanoparticle suspension, for example, insignificant change of the nanoparticle size or activity.

In some aspects, upon lyophilization and reconstitution of a protected suspension of nanoparticles, the average size of the nanoparticles can be within 10% of their size in the original composition, before lyophilization. In certain aspects, upon lyophilization and reconstitution of a protected suspension of nanoparticles, the average size of the nanoparticles can be within 5% of their size in the original composition, before lyophilization.

This disclosure contemplates lipid nanoparticle drug formulations containing, for example, siRNA agents, which can be prepared by lyophilization of a suspension of the nanoparticles, and reconstitution of the nanoparticles into a suspension after a period of storage. The reconstituted suspension can provide activity of the encapsulated agent, which is comparable to that of the suspension before lyophilization.

The reconstituted suspension, prepared after a period of storage, can contain lipid nanoparticles, which encapsulate the active agent and are comparable to the lipid nanoparticles before lyophilization.

In certain embodiments, the reconstituted suspension, prepared after a period of storage, can provide activity of the encapsulated agent, which is comparable to that of the suspension before lyophilization.

In further aspects, the reconstituted suspension, prepared after a period of storage, can provide stable nanoparticles comparable to that of the suspension before lyophilization. In certain aspects, the average particle size of the nanoparticles can be nearly equal to the size of the nanoparticles in the suspension before lyophilization.

### Pre-Lyophilization Lipid Nanoparticle Formulations

Embodiments of this disclosure can provide compositions of lipid nanoparticles, which compositions contain a protectant compound for a lyophilization process. The lipid nanoparticles can have any composition known in the art. The lipid nanoparticles may be synthesized and loaded with encapsulated cargo by any process, including processes known in the art. In some embodiments, the lipid nanoparticles can be prepared by a submersion injection process. Some examples of processes for lipid nanoparticles are given in US 2013/0115274. Some examples for preparing liposomes are given in Szoka, Ann. Rev. Biophys. Bioeng. 9:467 (1980); Liposomes, Marc J. Ostro, ed., Marcel Dekker, Inc., New York, 1983, Chapter 1.

In general, lipid nanoparticles can be synthesized by mixing lipid components in an organic solvent with an aqueous buffer solution containing active nucleic acid agents. The liposomes can be sized by filtration or extrusion. The liposome suspension or solution may be further transformed by diafiltration.

A lipid nanoparticle composition embodiment of this disclosure, which is stabilized for a lyophilization process, may contain lipid nanoparticles that encapsulate one or more active agents, such as nucleic acid agents, in a suspension. The suspension can be aqueous, and may contain a water-miscible solvent, such as ethanol. The composition, which is stabilized for a lyophilization process, may further contain protectant compounds to stabilize the liposomes in the lyophilization process.

The average size of lipid nanoparticles as synthesized can be from 40 nm to 120 nm, from 45 nm to 110 nm, from 45 nm to 80 nm, or from 45 nm to 65 nm.

The concentration of the active agent in a lipid nanoparticle composition of this disclosure can range from about 0.1 mg/mL to about 10 mg/mL. In some embodiments, the concentration of the active agent in a lipid nanoparticle composition of this disclosure can be from 0.5 mg/mL to 8 mg/mL, or from 1 mg/mL to 6 mg/mL, or from 2 mg/mL to 5 mg/mL, or from 3 mg/mL to 4 mg/mL.

Examples of protectant compounds include dextrin compounds. Examples of dextrin compounds include maltodextrins, and beta- and gamma-cyclodextrins.

Examples of dextrin compounds include methylated beta- and gamma-cyclodextrin compounds, and sulfoalkyl ether beta- and gamma-cyclodextrin compounds.

Examples of dextrin compounds include cyclodextrin compounds having one or more of the 2, 3 and 6 hydroxyl positions substituted with sulfoalkyl, benzenesulfoalkyl, acetoalkyl, hydroxyalkyl, hydroxyalkyl succinate, hydroxyalkyl malonate, hydroxyalkyl glutarate, hydroxyalkyl adipate, hydroxyalkyl, hydroxyalkyl maleate, hydroxyalkyl oxalate, hydroxyalkyl fumarate, hydroxyalkyl citrate, hydroxyalkyl tartrate, hydroxyalkyl malate, or hydroxyalkyl citraconate groups.

Examples of dextrin compounds include (2-hydroxypropyl)-β-cyclodextrin, 2-hydroxypropyl-3-cyclodextrin succinate, (2-hydroxypropyl)-γ-cyclodextrin, and 2-hydroxypropyl-γ-cyclodextrin succinate.

Examples of dextrin compounds include hydroxyethyl 3-cyclodextrin. Examples of dextrin compounds include dimethyl 3-cyclodextrin and trimethyl β-cyclodextrin. Examples of dextrin compounds include sulfobutyl ether 3-cyclodextrin and sulfobutyl ether γ-cyclodextrin. Examples of dextrin compounds include methyl-3-cyclodextrin and methyl-γ-cyclodextrin. Examples of dextrin compounds include hydroxypropyl-sulfobutyl-β-cyclodextrin. Examples of dextrin compounds include H107 SIGMA cyclodextrin (Sigma-Aldrich Corp.). Examples of dextrin compounds include CAVAMAX, CAVASOL, and CAVATRON cyclodextrins (Ashland Inc.). Examples of dextrin compounds include KLEPTOSE and CRYSMEB cyclodextrins (Roquette America Inc.). Examples of dextrin compounds include CAPTISOL cyclodextrins (Ligand Pharmaceuticals, Inc.).

In some embodiments, examples of dextrin compounds include dextrin compounds attached to a polymer chain or network. For example, cyclodextrin molecules can be attached to polymers of polyacrylic acid. In further embodiments, cyclodextrin molecules can be linked together with cross linking compounds such as acryloyl groups. In certain embodiments, vinyl acrylate hydrogel forms with attached cyclodextrin compounds can be used.

In some aspects, a dextrin compound to be used in a lipid nanoparticle composition of this disclosure can be combined with an adsorbate compound before being introduced into the lipid nanoparticle composition. Without wishing to be bound by any one particular theory, the pre-adsorption of a sterol compound by the dextrin compound may form an inclusion complex that can prevent a loss of activity of the active agent in the reconstituted drug product.

Examples of adsorbate compounds include cholesterol, lanosterol, zymosterol, zymostenol, desmosterol, stigmastanol, dihydrolanosterol, 7-dehydrocholesterol. Examples of adsorbate compounds include pegylated cholesterols, and cholestane 3-oxo-(C1-22)acyl compounds, for example, cholesteryl acetate, cholesteryl arachidonate, cholesteryl butyrate, cholesteryl hexanoate, cholesteryl myristate, cholesteryl palmitate, cholesteryl behenate, cholesteryl stearate, cholesteryl caprylate, cholesteryl n-decanoate, cholesteryl dodecanoate, cholesteryl nervonate, cholesteryl pelargonate, cholesteryl n-valerate, cholesteryl oleate, cholesteryl elaidate, cholesteryl erucate, cholesteryl heptanoate, cholesteryl linolelaidate, and cholesteryl linoleate.

Examples of adsorbate compounds include phytosterols, beta-sitosterol, campesterol, ergosterol, brassicasterol, delta-7-stigmasterol, and delta-7-avenasterol. Additional examples of protectant compounds include saccharide compounds. Examples of saccharide compounds include sugar compounds. Examples of protectant sugar compounds include monosaccharides such as C(5-6) aldoses and ketoses, as well as disaccharides such as sucrose, lactose, lactulose, maltose, trehalose, cellobiose, kojibiose, sakebiose, isomaltose, sophorose, laminaribiose, gentiobiose, turanose, maltulose, isomaltulose, gentiobiulose, mannobiose, melibiose, melibiulose, and xylobiose. Examples of protectant saccharide compounds include polysaccharides such as ficoll. The concentration of protectant compounds in the pre-lyophilization formulation can be from about 1% (w/v) to about 25% (w/v). In some embodiments, the concentration of protectant compounds in the pre-lyophilization formulation can be from 2% (w/v) to 20% (w/v), or from 4% (w/v) to 16% (w/v), or from 5% (w/v) to 15% (w/v), or from 6% (w/v) to 14% (w/v), or from 8% (w/v) to 12% (w/v). In certain embodiments, the concentration of protectant compounds in the pre-lyophilization formulation can be 6% (w/v), or 8% (w/v), or 10% (w/v), or 12% (w/v), or 14% (w/v), or 16% (w/v), or 18% (w/v), or 20% (w/v), or 22% (w/v), or 24% (w/v).

### Lyophilization Processes

Lyophilization processes can be carried out in any suitable vessel, such as glass vessels, or, for example, glass vials, or dual-chamber vessels, as are known in the pharmaceutical arts.

A stabilized lipid nanoparticle composition of this disclosure containing a protectant compound can be introduced into to the glass vessel. The volume of the composition added to the vessel can be from 0.1-20 mL, or from 1-10 mL.

Any lyophilization process can be used, including those known in the pharmaceutical arts. See, e.g., Remington's Pharmaceutical Sciences, 18th Ed., Mack Publishing Co., Easton, Pa. (1990).

The lyophilization process can include freezing the protectant-stabilized lipid nanoparticle composition at a temperature of from about −40° C. to about −30° C. The frozen composition can be dried form a lyophilized composition.

In some embodiments, the freezing step can ramp the temperature from ambient to the final temperature over several minutes. The temperature ramp can be about 1° C./minute.

In some embodiments, the drying step can be performed at a pressure in a range of about 0-250 mTorr, or 50-150 mTorr, at a temperature of from about −15° C. to about −38° C. The drying step can be continued at a higher temperature, up to ambient temperature, over a period of up to several days. The level of residual water in the solid lyophile can be less than about 5%, or less than 4%, or less than 3%, or less than 2%, or less than 1% (w/v).

The protectant-stabilized lipid nanoparticle compositions of embodiments of this disclosure, after lyophilization, can be reconstituted by methods known in the pharmaceutical arts. In some aspects, this disclosure provides methods for inhibiting the level of aggregated particles in a reconstituted drug product, made from a protectant-stabilized lipid nanoparticle composition of this disclosure after lyophilization. In some embodiments, the reconstituted drug product, made from a protectant-stabilized lipid nanoparticle composition of this disclosure after lyophilization, can have reduced levels of aggregate particles. In certain embodiments, the reconstituted drug product, made from a protectant-stabilized lipid nanoparticle composition of this disclosure after lyophilization, can have reduced levels of aggregate particles with a size greater than about 0.2 μm, or greater than about 0.5 μm, or greater than about 1 μm.

### Reconstituted Drug Product

The lyophile can be reconstituted in a pharmaceutically acceptable carrier. Examples of a pharmaceutically acceptable carrier include sterile water, water for injection, sterile normal saline, bacteriostatic water for injection, and a nebulizer solution. Examples of a pharmaceutically acceptable carrier include a pharmaceutically acceptable solution. Examples of a pharmaceutically acceptable solution include HEPES buffer, phosphate buffers, citrate buffers, and a buffer containing Tris(hydroxymethyl)aminomethane. Examples of a pharmaceutically acceptable solutions include pharmaceutically acceptable buffer solutions. Examples of a pharmaceutically acceptable solution include buffer solutions of maleic acid, tartaric acid, lactic acid, acetic acid, sodium bicarbonate, and glycine.

The reconstituted lyophile can be used as a drug product. The reconstituted lyophile can be further diluted with isotonic saline or other excipients to provide a predetermined concentration for administration. Examples of excipients include tonicifiers. Examples of excipients include stabilizers such as human serum albumin, bovine serum albumin, a-casein, globulins, a-lactalbumin, LDH, lysozyme, myoglobin, ovalbumin, and RNase A. Examples of excipients include buffers such as potassium acetate, sodium acetate, and sodium bicarbonate. Examples of excipients include amino acids such as glycine, alanines, arginine, betaine, leucine, lysine, glutamic acid, aspartic acid, histidine, proline, 4-hydroxyproline, sarcosine, γ-aminobutyric acid, alanopine, octopine, strombine, and trimethylamine N-oxide. Examples of excipients include non-ionic surfactants such as polysorbate 20, polysorbate 80, and poloxamer 407.

Examples of excipients include dispersing agents such as phosphotidyl choline, ethanolamine, acethyltryptophanate, polyethylene glycol, polyvinylpyrrolidone, ethylene glycol, glycerin, glycerol, propylene glycol, sorbitol, xylitol, dextran, and gelatin. Examples of excipients include antioxidants such as ascorbic acid, cysteine, thioglycerol, thioglycolic acid, thiosorbitol, and glutathione. Examples of excipients include reducing agents such as dithiothreitol, thiols, and thiophenes. Examples of excipients include chelating agents such as EDTA, EGTA, glutamic acid, and aspartic acid.

In some embodiments, the lyophile can be reconstituted using a syringe needle through a stoppered vial. The lyophile can be reconstituted with or without shaking the vial. The time for reconstitution can be from 3-30 seconds, or longer. In some embodiments, the reconstituted nucleic acid drug product can have less than 0.001% (w/v) of aggregate particles with a size greater than 0.2 μm. In certain aspects, the reconstituted nucleic acid drug product can have reduced cytokine activation.

In additional aspects, the nucleic acid drug product can be reconstituted after a storage time period of six months and retain 80% activity of the nucleic acid agents. In some embodiments, the nucleic acid drug product can be reconstituted after a storage time period of six months and the average particle size of the lipid nanoparticles can be less than 25% greater than before lyophilization. In certain embodiments, the nucleic acid drug product can be reconstituted after a storage time period of 24 months and retain 90% activity of the nucleic acid agents. In further embodiments, the nucleic acid drug product can be reconstituted after a storage time period of 24 months and the average particle size of the lipid nanoparticles can be less than 25% greater than before lyophilization.

### Methods for Modulating Coronavirus and Treating or Inhibiting Lung Diseases Such as MERS, SARS and Covid-19

Embodiments of this disclosure can provide RNAi molecules or siRNAs that can be used to down regulate or inhibit the expression of Coronavirus and/or Coronavirus proteins. In some embodiments, an RNAi molecule or siRNA of this disclosure can be used to down regulate or inhibit the expression of Coronavirus and/or Coronavirus proteins arising from Coronavirus haplotype polymorphisms that may be associated with a disease or condition such as MERS, SARS and/or Covid-19.

Monitoring or measuring of Coronavirus protein or mRNA levels can be used to characterize gene silencing, and to determine the efficacy of compounds and compositions of this disclosure.

The RNAi molecules or siRNAs of this disclosure can be used individually, or in combination with other siRNAs for modulating the expression of one or more genes. The RNAi molecules or siRNAs of this disclosure can be used individually, or in combination, or in conjunction with other known drugs for preventing or treating diseases or ameliorating symptoms of conditions or disorders associated with Coronavirus, including lung diseases such as MERS, SARS and/or Covid-19.

The RNAi molecules or siRNAs of this disclosure can be used to modulate or inhibit the expression of Coronavirus in a sequence-specific manner. The RNAi molecules or siRNAs of this disclosure can include a guide strand for which a series of contiguous nucleotides are at least partially complementary to a Coronavirus mRNA.

Treatment or inhibition of a lung disease such as MERS, SARS and/or Covid-19 may be characterized in suitable cell-based models, as well as ex vivo or in vivo animal models. Treatment or inhibition of a lung disease such as MERS, SARS and/or Covid-19 may be characterized by determining the level of Coronavirus mRNA or the level of Coronavirus protein in cells of affected tissue. Treatment or inhibition of a lung disease such as MERS, SARS and/or Covid-19 may be characterized by non-invasive medical scanning of an affected organ or tissue.

Embodiments of this disclosure may include methods for preventing, treating, inhibiting, or ameliorating the symptoms of a Coronavirus associated disease or condition in a subject in need thereof. In some embodiments, methods for preventing, treating, inhibiting or ameliorating the symptoms of a lung disease such as MERS, SARS and/or Covid-19 in a subject can include administering to the subject an RNAi molecule or siRNA of this disclosure to modulate the expression of a Coronavirus gene in the subject or organism. In some embodiments, this disclosure contemplates methods for down regulating the expression of a Coronavirus gene in a cell or organism, by contacting the cell or organism with an RNAi molecule or siRNA of this disclosure.

Embodiments of this disclosure encompass siRNA molecules of Tables 3-5 with nucleotides that are modified according to the examples above.

Some embodiments relate to a pharmaceutical composition comprising a dsRNA or siRNA and a pharmaceutically acceptable carrier. In some embodiments, the carrier comprises a lipid or liposome. Some embodiments relate to a vector comprising the dsRNA, siRNA or pharmaceutical composition. Some embodiments relate to a cell comprising the dsRNA, siRNA, pharmaceutical composition, or vector. Examples of such cells include lung and nasal cells. In some examples, the vector includes a plasmid or other construct configured to express a dsRNA, siRNA, or RNAi molecule.

Some embodiments relate to a method for preventing, treating or ameliorating one or more symptoms of MERS, SARS and/or Covid-19 in a mammal in need thereof. In some embodiments, the method includes administering to the mammal a therapeutically effective amount of a composition comprising a specific inhibitor or antagonist of Coronavirus.

In some embodiments of the method, the specific inhibitor or antagonist of Coronavirus comprises a Coronavirus protein expression inhibitor. In some embodiments, the Coronavirus protein expression inhibitor comprises a Coronavirus siRNA. In some embodiments, the Coronavirus siRNA comprises the dsRNA. In some embodiments, the Coronavirus siRNA comprises a nanoparticle. In some embodiments, the symptoms of MERS, SARS and/or Covid-19 comprise a positive test for presence of a Coronavirus (e.g., in a sample obtained by nasal pharyngeal swab) as compared to a mammal without MERS, SARS and/or Covid-19. In other embodiments, the symptoms of MERS, SARS and/or Covid-19 comprise clinical observations such as cough, shortness of breath or difficulty breathing, fever, chills, muscle pain, sore throat, and/or new loss of taste or smell.

In some embodiments, the one or more symptoms of MERS, SARS and/or Covid-19 are prevented, treated, inhibited or ameliorated within 1 days, 3 days, 5 days, or 7 days, or within a time frame defined by a range set forth by any two of the aforementioned numbers of days, upon administration of the specific inhibitor or antagonist of a Coronavirus. In some embodiments, the one or more symptoms of MERS, SARS and/or Covid-19 are prevented, treated, or ameliorated within 1 day, 2 days, 3 days, 4 days, 5 days, 6 days, 7 days, 10 days, or 14 days, or within a range defined by any two of the aforementioned numbers of days after administration of the specific inhibitor or antagonist of a Coronavirus. In some embodiments, the one or more symptoms of MERS, SARS and/or Covid-19 are prevented, treated, or ameliorated for 1 day, 2 days, 3 days, 4 days, 5 days, 6 days, 7 days, 10 days, 14 days, 21 days, 28 days, 1 month, 2 months, 3 months, 4 months, 5 months, 6 months, 7 months, 8 months, 9 months, 10 months, 11 months, 12 months, or more, or by a range of time periods defined by any two of the aforementioned time periods, after administration of the specific inhibitor or antagonist of a Coronavirus.

In some embodiments, one or more symptoms of MERS, SARS and/or Covid-19 are ameliorated by at least 25%, 50%, 75%, or 100%, or by a range defined by any two of the aforementioned percentages, upon administration of the specific inhibitor or antagonist of a Coronavirus.

### EXAMPLE PROTOCOL

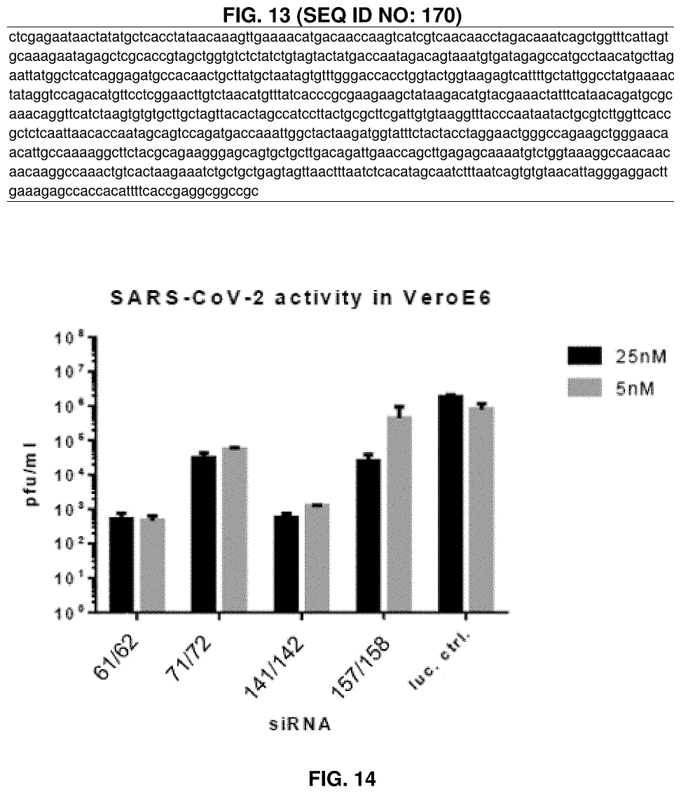

Example protocol for activity screening using a psiCHECK reporter assay. The psiCHECK2-COV reporter construct (psiCHECK2 AY535007, see FIG. 13 SEQ. ID. NO. 170) contains all siRNA targeting regions in sequential order with about 10 bp upstream and downstream of siRNA target sequence. Experimental daily schedule: Day 1, Cell seeding at 5×10³ cells/100 ul/well; Day 2, Co-transfection (cell confluency around 80%); Day 3, Cell harvest for luciferase activity measurement.

Platform: 96 wells plate

No. of wells/sample: Triplicate

[siRNA]: 5 pM, 50 pM & 500 pM

Cell line: HeLa

Incubation medium EMEM (containing 10% FBS)

Transfection agent: Lipofectamine 2000 (L2K)

Incubation condition: At 37° C. incubator under 5% CO₂

Harvest time: 24 hours after transfection

Reporter Assay Kit: Dual-Luciferase Reporter Assay (Promega, Cat #: E1960)

### Example Co-Transfection Procedure for 50 pM siRNA Concentration

1. Dilute 0.1 μL of 50 nM siRNA (final conc. 50 pM) in 2.9 μL Opti-MEM.

2. Dilute 0.1 uL of 100 ng/mL psiCHECK2 plasmid in 2.9 uL Opti-MEM.

3. Dilute 0.1 uL L2K in 3.9 μL Opti-MEM.

4. Combine the diluted siRNA and psiCHECK2 plasmid with the diluted L2K. Mix gently by tapping without pipetting and incubate for 15 min at RT.

5. During incubation aspirate culture media on dish and then replace with 90 μL EMEM.

6. Add the siRNA-plasmid-L2K complex drop by drop to a plate. This gives a final volume of 100 μL and a final siRNA concentration of 50 pM.

7. Mix gently by rocking the dish back and forth.

8. Incubate the cells overnight at 37° C. in a CO₂ incubator.

9. Measure luciferase activity using Promega's Luciferase Assay System (Promega, E4550) according to manufacturer protocol.

### Example Sequential Transfection Protocol to Determine siRNA Liposomal Formulation Knockdown Activity with psiCHECK Reporter System

Platform: 96 wells plate

No. of wells/sample: Duplicate

[siRNA] 5000 to 0.05243 nM (diluted in D-PBS, 1×) 2.5 fold serial dilutions

Incubation medium EMEM (containing 10% FBS)

Transfection agent: Lipofectamine 2000 (L2K)

Incubation condition: At 37° C. incubator under 5% CO2

Harvest time: 24 hours after transfection

### Transfection Procedure

1. Dilute COV psiCHECK plasmid in Opti-MEM.

2. Dilute L2K in Opti-MEM.

3. Combine the diluted psiCHECK plasmid with the diluted L2K. Mix gently by tapping without pipetting and incubate for 5 min at RT.

4. During incubation aspirate culture media on dish, rinse twice with PBS and then replace with 90 μL full EMEM culture medium.

5. Add 10 ul of plasmid/L2K complex drop by drop to a plate, giving the final volume of 100 μL.

6. Mix gently by rocking the dish.

7. Incubate the cells overnight at 37° C. in a CO₂ incubator.

8. Measure luciferase activity using Promega's Luciferase Assay System (Promega, E4550) according to manufacturer protocol.

Procedure: plate HeLa cells and incubate; transfect cells with reporter construct using Lipofectamine; remove medium with formulation (optional wash step); transfect cells with siRNA in formulation and incubate for 24 h; measure Luciferase activity.

### EXAMPLES

### Example 1—Lipid Nanoparticle Formulation Provides Enhanced Distribution to Lung In Vivo

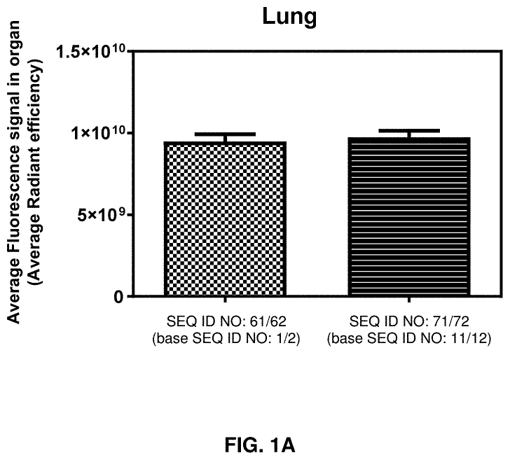

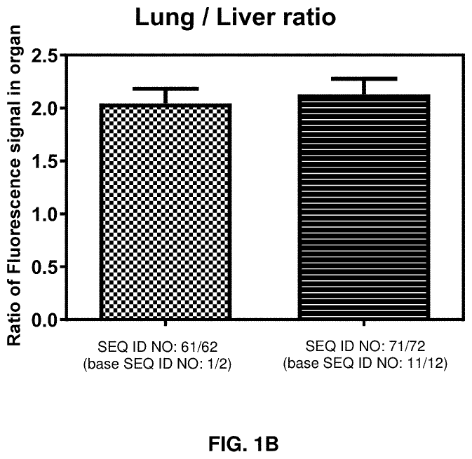

FIG. 1A shows enhanced distribution to lung in vivo mouse using embodiments of a pharmaceutical formulation that provides delivery of an API to the lung as described in International Application No. PCT/US2019/061702 and U.S. Patent Publication No. 2020/0157540, both of which are hereby incorporated herein by reference in their entireties and particularly for the purposes of describing such pharmaceutical formulations and methods for making them. The LNP pharmaceutical formulations contained an API (siRNA targeted to Coronavirus, (SEQ ID NOS: 61/62 or 71/72)), Compound A 25 mol %, cholesterol 30 mol %, DOPE 20 mol %, DOPC 20 mol %, and DSPE-mPEG-2000 5 mol %. Organs were harvested 4 hours after injection of a dose at 4 mg/kg in naïve animals, with 5 animals per group. The accumulation of siRNA in the organ was measured by fluorescence.

FIG. 1B shows the ratio of distribution of lung to liver in vivo mouse for embodiments of formulations of this invention. The administered LNP compositions of the formulations contained API (siRNA targeted to Coronavirus, (SEQ ID NOS: 61/62 or 71/72)), Compound A 25 mol %, cholesterol 30 mol %, DOPE 20 mol %, DOPC 20 mol %, and DSPE-mPEG-2000 5 mol %. The results showed that the ratio of the distribution of active agent to lung over liver in vivo mouse was 2-fold.

### Example 2—Small Scale siRNA Synthesis (SEQ ID NOS: 61-168)

Each single strand was synthesized with the corresponding NPHL solid support using Mermade 12 on 2 μmol scale. Each single strand was then cleaved from the solid support and desilylated by methylamine and TEA.3HF, respectively. Purification of the crude single strand was achieved by ion-exchange chromatography. After desalting by a size-exclusion column, a pair of complementary strands was finally annealed and lyophilized to yield the targeted duplex (usually with a yield of 1-2 mg).

### Example 3—SARS-CoV siRNAs Downregulate SARS-CoV mRNA In Vitro

As disclosed herein, a psiCHECK/siRNA co-transfection method was used to characterize the potency and dose dependency of SARS-CoV mRNA inhibition by siRNA using HeLa cell line. Thirty double-stranded RNA described in Table 3 (SEQ ID NOS: 1/2-59/60) were designed and thirty corresponding synthetic, double-stranded RNA with mUmU overhangs described in Example 2 and Table 4 (SEQ ID NOS: 61/62-119/120) were used to temporarily inhibit the expression SARS-CoV at the mRNA level. HeLa cells were obtained from ATCC (Manassas, Va., Cat #CCL-2), maintained in EMEM (Thermo Fisher Scientific Inc.), supplemented with 10% FBS, and incubated at 37 C with 5% CO2. After incubation, HeLa cells were seeded at 5×10\hat{ }3 cells per well into 96-well plates. After 24 hours of culture, cells were transfected with 10 ng/mL psiCHECK-2 or co-transfected with psiCHECK-2 and SARS-CoV-siRNA at 5 pM, 50 pM, and 500 pM of siRNA using Lipofectamine 2000 (Thermo Fisher Scientific Inc. Cat #11667027). The psiCHECK reporter construct (psiCHECK2 Ay535007, BlueHeron Inc.) contains all 30 siRNA candidate regions in sequential order with about 10 bp upstream and downstream of siRNA target sequence of the coronavirus. The psiCHECK-2 plasmid contains all the siRNAs sequences which were cloned into the 3′ UTR of Renilla luciferase reporter gene. Also included was an internal control plasmid, which contained the firefly luciferase reporter gene.

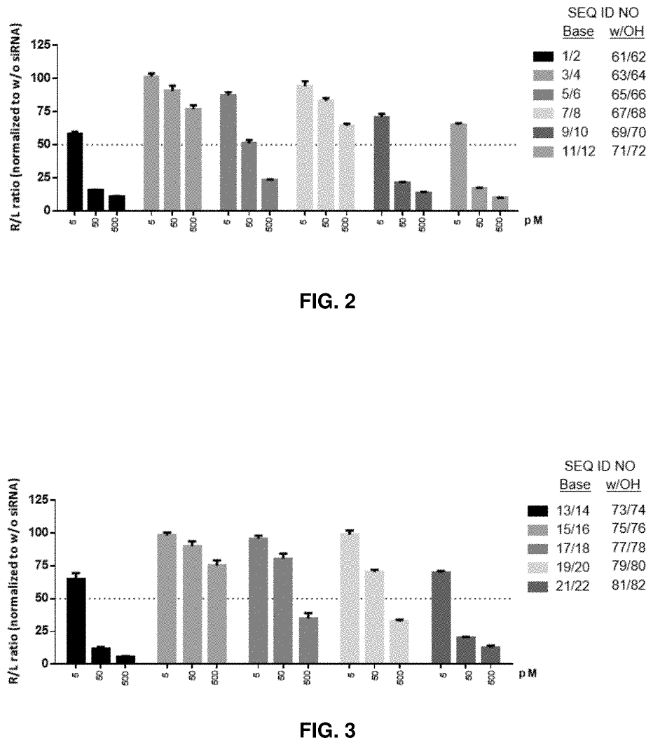

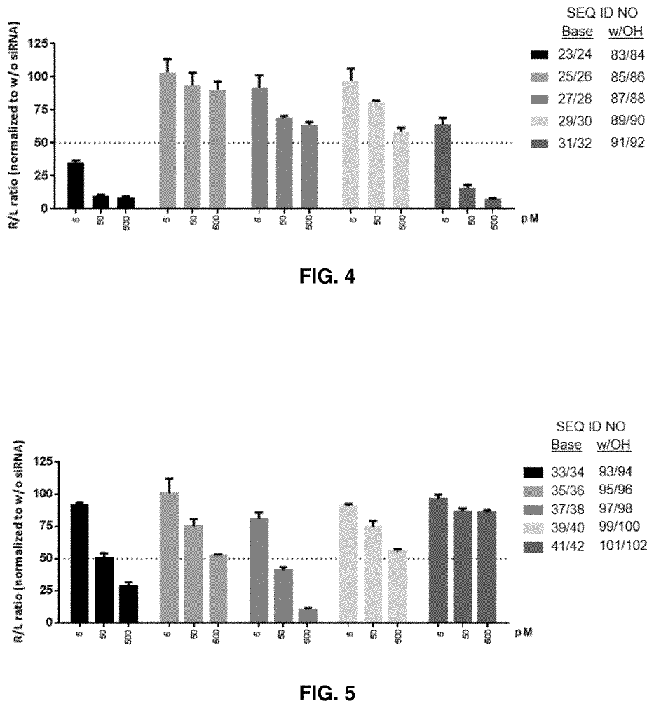

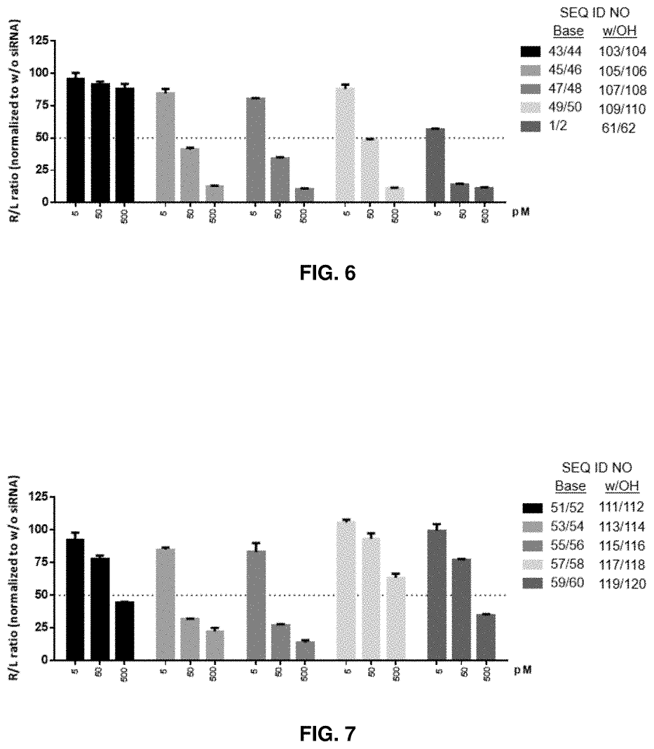

Twenty-four hours following transfection, the cells were lysed. The SARS-CoV mRNA knockdown activity of SARS-CoV siRNAs was evaluated by measuring the reduction of luciferase activity in HeLa cells through a Dual-Luciferase Reporter (DLR) Assay System (Promega Corp. Cat #E1980) according to the manufacturer's protocol. Renilla luciferase was normalized to firefly luciferase activity. From the 30 siRNA tested, six pan coronavirus siRNAs targeted all three forms of coronavirus (SARS-CoV, SARS-CoV-2, and MERS-CoV). From these six, three siRNA (SEQ ID NOS: 61/62, 69/70, and 71/72) down regulated SARS-CoV mRNA by ˜90% at 500 pM concentration (FIGS. 2-7, Table 7). SEQ ID NO: 61/62 targeted SARS-CoV-2 sequence at the RNA-dependent RNA polymerase region; SEQ ID NOS: 69/70 and 71/72 targeted 3′-to-5′ exonuclease gene. SEQ ID NOS: 73/74, 81/82, 83/84 and 91/92 were among the most potent SARS-CoV siRNAs with >90% mRNA down regulation when used at 500 pM. The results confirmed picomolar range IC50 potency of SARS-CoV siRNAs in HeLa cells.

TABLE 7
Downregulation of SARS-CoV mRNA
through SARS-CoV siRNAs

| Sequence with 3′ | | | |
|---|---|---|---|
| mU overhang | | | |
| SEQ ID NO | | abs EC50 | % at |
| Sense | Antisense | (nM) | 0.5 nM |
| 61 | 62 | 0.007 | 11.2 |
| 63 | 64 | N/A | 76.9 |
| 65 | 66 | 0.053 | 23.4 |
| 67 | 68 | N/A | 64.4 |
| 69 | 70 | 0.011 | 13.9 |
| 71 | 72 | 0.009 | 10.0 |
| 73 | 74 | 0.008 | 5.6 |
| 75 | 76 | N/A | 75.2 |
| 77 | 78 | 0.273 | 34.8 |
| 79 | 80 | 0.107 | 32.5 |
| 81 | 82 | 0.010 | 12.4 |
| 83 | 84 | 0.003 | 8.7 |
| 85 | 86 | N/A | 90.1 |
| 87 | 88 | N/A | 63.6 |
| 89 | 90 | N/A | 58.4 |
| 91 | 92 | 0.008 | 7.8 |
| 93 | 94 | 0.051 | 28.6 |
| 95 | 96 | N/A | 52.2 |
| 97 | 98 | 0.031 | 10.6 |
| 99 | 100 | N/A | 55.6 |
| 101 | 102 | N/A | 86.1 |
| 103 | 104 | N/A | 87.8 |
| 105 | 106 | 0.033 | 12.3 |
| 107 | 108 | 0.023 | 10.7 |
| 109 | 110 | 0.046 | 10.8 |
| 111 | 112 | 0.376 | 44.5 |
| 113 | 114 | 0.020 | 22.3 |
| 115 | 116 | 0.018 | 13.8 |
| 117 | 118 | N/A | 63.5 |
| 119 | 120 | 0.131 | 34.4 |

Among the siRNA tested, SEQ ID NOS: 61/62 and 71/72 were identified as very potent with single digit pM range IC50 (6 pM and 9 pM respectively).

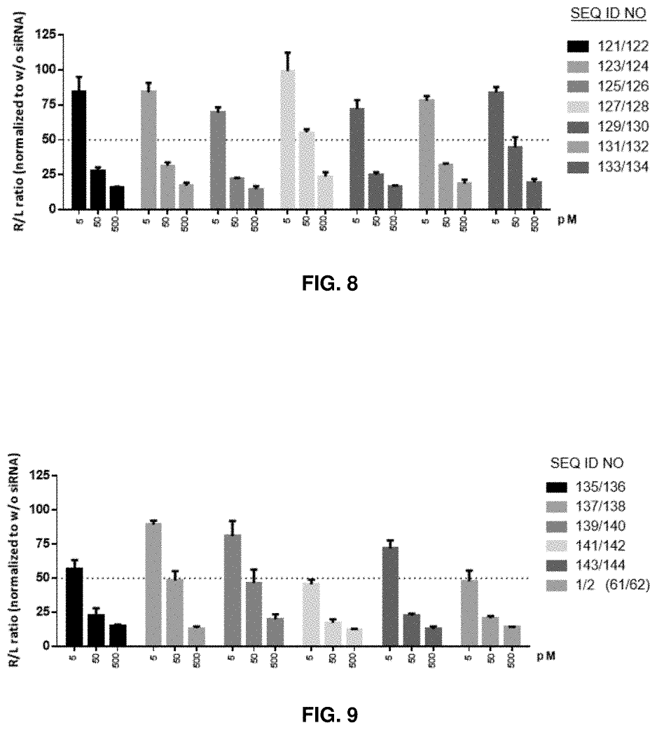

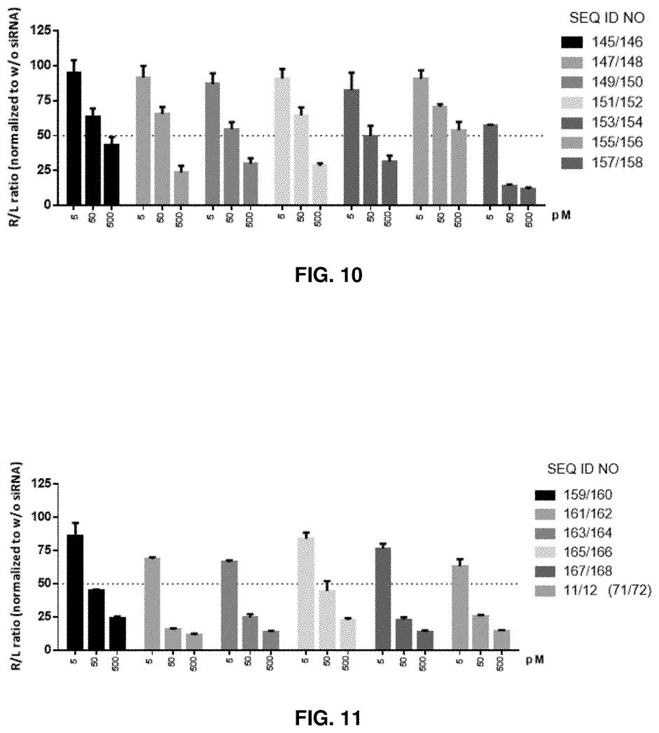

As disclosed herein, 12 modified siRNAs (SEQ ID NOS: 121/122, 123/124, 125/126, 127/128, 129/130, 131/132, 133/134, 135/136, 137/138, 139/140, 141/142 and 143/144) were designed from SEQ ID NO: 61/62, and 12 modified siRNAs (SEQ ID NOS: 145/146, 147/148, 149/150, 151/152, 153/154, 155/156, 157/158, 159/160, 161/162, 163/164, 165/166, and 167/168) were designed from SEQ ID NO 71/72. Of the modified siRNAs based on SEQ ID NO: 61/62, SEQ ID NOS 125/126, 135/136, 141/142 and 143/144 retained strong activity and potency and resulted in comparable IC50 to SEQ ID NO: 61/62 (IC50: 0.003 to 0.009 nM) (FIGS. 8 and 9, Table 8). When the modified siRNAs based on SEQ ID NO: 71/72 were tested for their activity on down-regulating the SARS-CoV mRNA, SEQ ID NOS: 157/158, 161/162, 163/164 and 167/168 maintained similar potency and demonstrated comparable IC50 to the original SEQ ID NO: 71/72 (IC50: 0.005 to 0.010 nM) (FIGS. 10 and 11, Table 9). Importantly, all tested siRNAs successfully down-regulated the mRNA of coronavirus in a dose-dependent manner. This strongly evidences the potential effectiveness of the disclosed siRNAs as a therapeutic strategy for SARS-CoV-2 pathogenesis.

TABLE 8
Downregulation of SARS-CoV mRNA through
siRNAs based on modifications of SEQ ID NO: 61/62

| Sequence | | rel | | |
|---|---|---|---|---|
| SEQ ID NO | | IC50 | abs EC50 | % at |
| Sense | Antisense | (nM) | (nM) | 0.5 nM |
| 61 | 62 | 0.003 | 0.004 | 14.3 |
| 121 | 122 | 0.015 | 0.019 | 15.7 |
| 123 | 124 | 0.016 | 0.021 | 17.2 |
| 125 | 126 | 0.008 | 0.010 | 14.7 |
| 127 | 128 | 0.043 | 0.057 | 23.8 |
| 129 | 130 | 0.009 | 0.012 | 16.4 |
| 131 | 132 | 0.013 | 0.018 | 18.7 |
| 133 | 134 | 0.026 | 0.037 | 19.5 |
| 135 | 136 | 0.005 | 0.007 | 15.0 |
| 137 | 138 | 0.041 | 0.046 | 13.3 |
| 139 | 140 | 0.030 | 0.040 | 19.9 |
| 141 | 142 | 0.003 | 0.004 | 12.4 |
| 143 | 144 | 0.009 | 0.012 | 12.9 |

TABLE 9
Downregulation of SARS-CoV mRNA through
siRNAs based on modifications of SEQ ID NO: 71/72

| Sequence | | rel | | |
|---|---|---|---|---|
| SEQ ID NO | | IC50 | abs EC50 | % at |
| Sense | Antisense | (nM) | (nM) | 0.5 nM |
| 71 | 72 | 0.007 | 0.010 | 14.6 |
| 145 | 146 | 0.033 | 0.141 | 43.4 |
| 147 | 148 | 0.126 | 0.115 | 23.6 |
| 149 | 150 | 0.033 | 0.068 | 29.8 |
| 151 | 152 | 0.090 | 0.116 | 28.4 |
| 153 | 154 | 0.020 | 0.049 | 31.4 |
| 155 | 156 | 0.036 | 0.042 | 53.8 |
| 157 | 158 | 0.005 | 0.006 | 11.6 |
| 159 | 160 | 0.023 | 0.038 | 24.3 |
| 161 | 162 | 0.007 | 0.009 | 11.3 |
| 163 | 164 | 0.008 | 0.011 | 13.7 |
| 165 | 166 | 0.023 | 0.037 | 22.7 |
| 167 | 168 | 0.010 | 0.013 | 13.9 |

### Example 4—SARS-CoV siRNAs Reduce Plaques Caused by SARS-CoV In Vitro

As disclosed herein, the siRNAs (SEQ ID NO: 61/62 and SEQ ID NO: 71/72 and their chemically modified derivatives SEQ ID NO: 141/142 and SEQ ID NO: 157/158 target all three forms of coronavirus (SARS-CoV, SARS-CoV-2, and MERS-CoV). These four siRNAs were evaluated for their effect on coronavirus replication reduction using a plaque assay on VERO-E6 cells. Plaque assays are considered the gold standard for monitoring viral infectivity; when a cell culture is contacted by a viral specimen, the plated cells forms plaques in numbers correlating with the infectivity of that virus.

VERO-E6 cells obtained from ATCC (Manassas, Va., Cat #CRL-1586) were maintained in EMEM (Thermo Fisher Scientific Inc.), supplemented with 10% FBS, and incubated at 37 C with 5% CO2. Following incubation, VERO-E6 cells were seeded at 1×10\hat{ }4 cells per well into 96-well plates and grown for 24 hours. VERO-E6 cells were then transfected with modified siRNA (SEQ ID NO: 141/142 or SEQ ID NO: 157/158) at 0.008 nM, 0.04 nM, 0.2 nM, 1 nM, 5 nM, and 25 nM of siRNA using RNAiMAX (Thermo Fisher Scientific Inc. Cat #13778075). The corresponding siRNAs from which they were derived (SEQ ID NO: 61/62 and SEQ ID NO: 71/72) were transfected at 5 nM and 25 nM. Twenty-four hours following transfection, the cells were infected with SARS-CoV-2 at multiplicity of infection (MOI) 0.5 for 24 hours at 37 C and 5% CO2 with rocking every 15 minutes. The virus was removed through washing once with PBS, following by an additional wash with Vero growth media (DMEM+10% FBS+1×Pen/Strep). Cells were resuspended in Vero growth media and incubated for 24 hours at 37 C 5% CO2. After 24 h, supernatants were removed and frozen at −80 C for quantification by plaque assay. In addition, infected cells were collected in TRIzol for RNA analysis. RNA was isolated from the infected cells followed by qRT-PCR to quantify the reduction of viral mRNA.

As disclosed herein, plaque assays were performed using the standard protocol. In brief, 10-fold serial dilutions of viral samples in DMEM (no serum) containing SARS-CoV-2 were adsorbed onto a monolayer of cells. After adsorption, a liquid overlay medium (L-OM) was applied to the cell monolayer to restrict virus growth. In addition, instead of incorporating a vital stain into the secondary overlay (S-OM), the L-OM is removed from the monolayer, fixed, and stained with crystal violet. Plates were incubated for 2 days. Cells were then fixed overnight by filling wells with 10% formaldehyde in PBS. The agarose overlay was removed and stained 10 min with 0.025% crystal violet in 2% EtOH. Then wells were rinsed with water and plaques counted. Plaques were counted from the dilution plate having 5 to 50 plaques and then pfu/ml calculated and corrected for baseline.

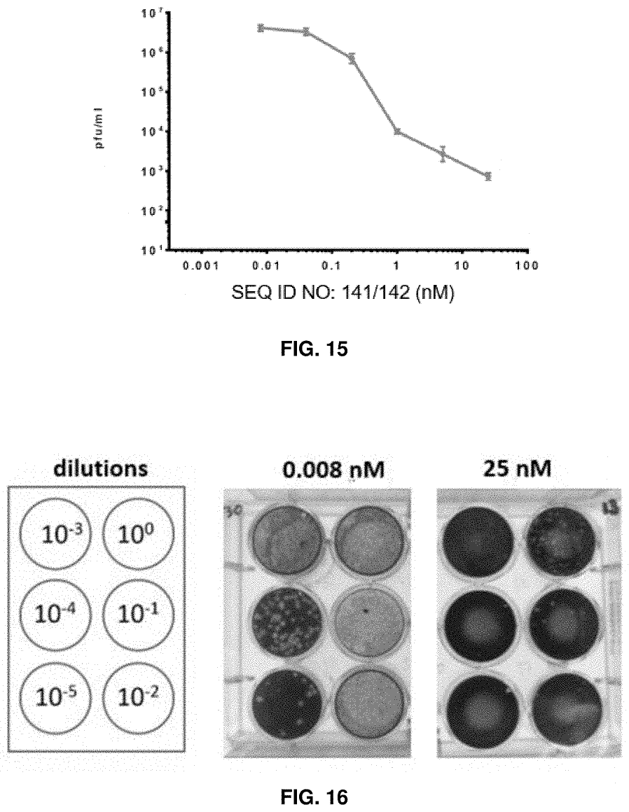

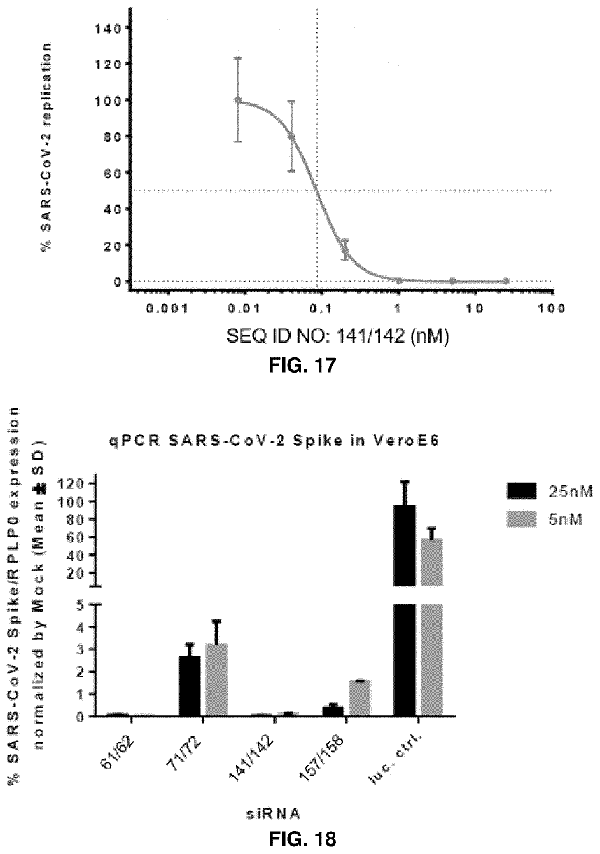

A modified siRNA (SEQ ID NO: 141/142) and its parental siRNA (SEQ ID NO: 61/62) were the most potent sequences tested at 25 and 5 nM, respectively (FIG. 14). At 24 hours, there was a >99% reduction in viral RNA at 1 nM and above present in the supernatant (indicative of released virions) of samples treated with SEQ ID NO: 141/142. A 3- to 4-log reduction of viral titer was observed, with a calculated IC50 of 86 pM (FIG. 15-17).

As disclosed herein, quantitative real time PCR (qRT-PCR) was performed to assess the changes in SARS-CoV-2 gene expression upon treatment with siRNAs using the standard protocol. In brief, cDNA was prepared from transfected cells utilizing BioRad iScript cDNA synthesis kit per the manufacturer's protocol. Expression analysis conducted with the standard ΔΔCT to determine the relative level of expression of the SARS-CoV-2 spike protein compared to the RPLP0 (60S acidic ribosomal protein P0) housekeeping gene for all samples.

Prime sequence:
(SEQ ID NO: 171)
RPLP0 F-GTGTTCGACAATGGCAGCAT;
(SEQ ID NO: 172)
RPLP0 R-GACACCCTCCAGGAAGCGA
SARS-CoV-2
(SEQ ID NO: 173)
Spike F-CCTACTAAATTAAATGATCTCTGCTTTACT
SARS-CoV-2
(SEQ ID NO: 174)
Spike R-CAAGCTATAACGCAGCCTGTA

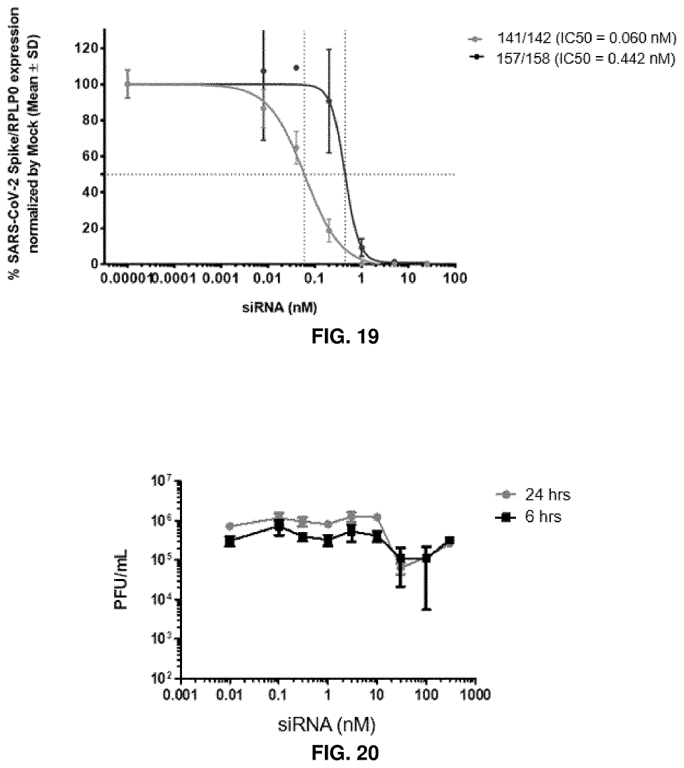

Once again, the modified derivative siRNA SEQ ID NO: 141/142 and the siRNA on which it was based (SEQ ID NO: 61/62) were the most potent sequences tested at 25 and 5 nM, respectively (FIG. 18). A >99% reduction in viral spike protein RNA was observed in samples treated with SEQ ID NO: 141/142 at 1 nM and above, with a calculated IC50 of 60 pM (FIG. 19).

Taken together these results demonstrate that SEQ ID NO: 141/142 has antiviral activity against the SARS-CoV-2 clinical isolate in vitro and is a particularly useful antiviral approach to limit SARS-CoV-2 in patients with Covid-19. KD data from reporter assay (see Table 7 for unmodified sequences and Tables 8 and 9 for data on modified versions) have been demonstrated along with infection assay data (FIGS. 14-19). All the exemplified siRNA are very potent in KD reporter assay with IC50 below 10 pM. Surprisingly, in the infection assay, SEQ ID NO: 61/62 (based on unmodified SEQ ID NO: 1/2) and its modified derivative (SEQ ID NO: 141/142) are unexpectedly superior to SEQ ID NO: 71/72 (based on unmodified SEQ ID NO: 11/12) and its modified derivative SEQ ID NO: 157/158, respectively.

### Example 5—Lipid Nanoparticle Formulation with Modified siRNA Provides Enhanced Coronavirus Inhibition

As disclosed herein, the effectiveness of SEQ ID NO: 141/142 against coronavirus infection was assessed as a component of a lipid nanoparticle (LNP) formulation. This formulation was composed of five lipids: Compound A, DOPE, DOPC, Cholesterol, and DSPE-mPEG-2000 at a molar ratio of [25:20:20:30:5]. These lipids were selected based on their low toxicity profile and ability to form a stable drug delivery system for distribution into the lungs.

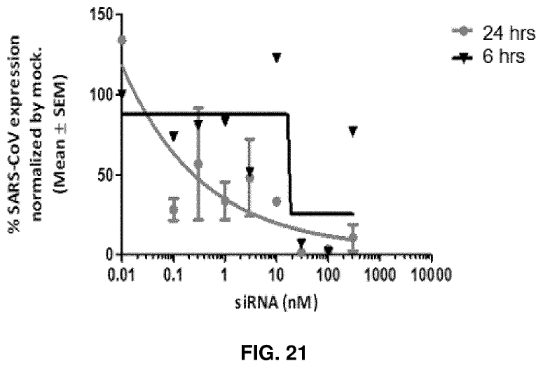

VERO-E6 cells were transfected with 0.1 nM to 300 nM LNP formulation containing the SARS-CoV siRNA, SEQ ID NO: 141/142. The cells were simultaneously incubated with 5 μg/mL PLA2 to hydrolyze DSPE-mPEG-2000. Either 6 or 24 hours following transfection, cells were infected with SARS-CoV-2 (isolate USA-WA1/2020) at MOI 0.5 for 24 hours. Supernatants were then removed and frozen at −80 C for quantification by plaque assay. RNA was isolated from the infected cells followed by qRT-PCR to quantify the reduction of viral mRNA. From the plaque assay, it was observed that 30 nM and 100 nM of the LNP formulation containing SEQ ID NO: 141/142 were most effective, with a 1.5-log reduction of viral titer was observed (FIG. 20). From qRT-PCR, a >90% reduction in viral spike protein RNA was observed in samples treated with the LNP formulation containing SEQ ID NO: 141/142 at 30 nM and above (FIG. 21). In sum, these results demonstrate that the LNP formulation containing SEQ ID NO: 141/142 has antiviral activity against the SARS-CoV-2 clinical isolate and is a useful antiviral approach for patients with Covid-19.

The term “comprising” as used herein is synonymous with “including,” “containing,” or “characterized by,” and is inclusive or open-ended and does not exclude additional, unrecited elements or method steps.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific embodiments disclosed herein, but that it cover all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A double-stranded ribonucleic acid (dsRNA) for inhibiting expression of a Coronavirus protein, comprising a sense strand and an antisense strand comprising a region of complementarity complementary to an mRNA encoding the protein, wherein the strands form a duplex region, and wherein the antisense strand is at least 15 contiguous nucleotides from any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, or has at least 90%, 91%, 92%, 93%, 94%, 95%, 96%, 97%, 98%, or 99% sequence identity to any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, or a sequence identity to any one or more of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166 or 168, which is within a range defined by any two of the aforementioned percentages.

2. (canceled)

3. (canceled)

4. (canceled)

5. The dsRNA of claim 1, wherein the sense strand comprises a nucleic acid sequence in accordance with SEQ ID NO: 1, and wherein the antisense strand comprises a nucleic acid sequence in accordance with SEQ ID NO: 2.

6. (canceled)

7. The dsRNA of claim 1, further comprising one or more single-stranded overhang(s) of one or more nucleotides.

8. (canceled)

9. (canceled)

10. (canceled)

11. (canceled)

12. (canceled)

13. (canceled)

14. The dsRNA of claim 1, wherein the sense strand comprises a nucleic acid sequence in accordance with SEQ ID NO: 61, and wherein the antisense strand comprises a nucleic acid sequence in accordance with SEQ ID NO: 62.

15. (canceled)

16. The dsRNA of claim 1, comprising at least one modified nucleotide, wherein said modified nucleotide is selected from the group of: a 2′-O-methyl modified nucleotide, a nucleotide comprising a 5′-phosphorothioate group, a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural base comprising nucleotide.

17. (canceled)

18. (canceled)

19. (canceled)

20. (canceled)

21. The dsRNA of claim 1, wherein the sense strand comprises a nucleic acid sequence in accordance with SEQ ID NO: 141, and wherein the antisense strand comprises a nucleic acid sequence in accordance with SEQ ID NO: 142.

22. (canceled)

23. (canceled)

24. A pharmaceutical composition comprising the dsRNA of claim 1 and a pharmaceutically acceptable carrier.

25. (canceled)

26. The pharmaceutical composition of claim 24, comprising:
(a) an effective amount of the dsRNA;
(b) a compound having the following Formula II: 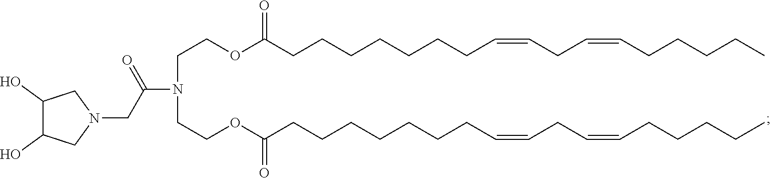 [SMILES: CCCCC/C=C\C/C=C\CCCCCCCC(=O)OCCN(CCOC(=O)CCCCCCC/C=C\C/C=C\CCCCC)C(=O)CN1CC(O)C(O)C1]
(c) a DSPE lipid comprising a polyethyleneglycol (PEG) region, a multi-branched PEG region, a methoxypolyethyleneglycol (mPEG) region, a carbonyl-methoxypolyethyleneglycol region, or a polyglycerine region;
(d) a sterol lipid; and
(e) one or more neutral lipids.

27. The pharmaceutical composition of claim 26, wherein the compound of Formula II is Compound A, and the compound of Formula A is from 15 mol % to 35 mol % of the total lipids of the composition:

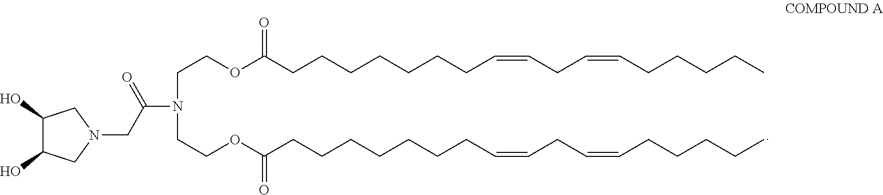 [SMILES: CCCCC/C=C\C/C=C\CCCCCCCC(=O)OCCN(CCOC(=O)CCCCCCC/C=C\C/C=C\CCCCC)C(=O)CN1C[C@@H](O)[C@@H](O)C1]

28. (canceled)

29. (canceled)

30. The pharmaceutical composition of claim 26, wherein the sterol lipid is from 25 mol % to 40 mol % of the total lipids of the composition and the sterol lipid is cholesterol.

31. (canceled)

32. (canceled)

33. The pharmaceutical composition of claim 26, wherein the DSPE lipid is from 1 mol % to 8 mol % of the total lipids of the composition, and the DSPE lipid is DSPE-mPEG-2000.

34. (canceled)

35. (canceled)

36. (canceled)

37. (canceled)

38. The pharmaceutical composition of claim 26, wherein the sum of the one or more neutral lipids is from 25 mol % to 45 mol % of the total lipids of the composition, and wherein each of the neutral lipids individually is from 5 mol % to 40% mol %, wherein the one or more neutral lipids are 1,2-dioleoyl-sn-Glycero-3-Phosphocholine (DOPC) and 1,2-dioleoyl-sn-glycero-3-phosphoethanolamine (DOPE).

39. (canceled)

40. (canceled)

41. (canceled)

42. (canceled)

43. (canceled)

44. The pharmaceutical composition of claim 26, wherein:
the compound of Formula II comprises 15 mol % to 35 mol % of the total lipids of the composition;
cholesterol, DOPC, and DOPE combined comprise 50 mol % to 85 mol % of the total lipids of the composition;
DSPE-mPEG-2000 comprises from 1 mol % to 8 mol % of the total lipids of the composition; and
wherein the compound of Formula II, cholesterol, DOPC, DOPE, and DSPE-mPEG-2000 combined comprise at least 97 mol % of the total lipids of the composition.

45. (canceled)

46. (canceled)

47. (canceled)

48. The pharmaceutical composition of claim 24, wherein the composition comprises nanoparticles having a Z-average size of 30 nm to 100 nm, and the nanoparticles encapsulate the dsRNA.

49. (canceled)

50. (canceled)

51. The pharmaceutical composition of claim 27, wherein:
the dsRNA is selected from SEQ ID NO: 61/62, and SEQ ID NO: 141/142;
the DSPE lipid comprises DSPE-mPEG-2000;
the sterol lipid comprises cholesterol; and
the neutral lipids comprises at least one of DOPC and DOPE;
Compound A comprises 20 mol % to 30 mol % of the total lipids of the composition;
cholesterol comprises 25 mol % to 35 mol % of the total lipids of the composition;
DOPC and DOPE combined comprise 30 mol % to 50 mol % of the total lipids of the composition;
DSPE-mPEG-2000 comprises from 4 mol % to 6 mol % of the total lipids of the composition; and
wherein Compound A, cholesterol, DOPC, DOPE, and DSPE-mPEG-2000 combined comprise at least 97 mol % of the total lipids of the composition.

52. A pharmaceutical solution comprising solvents ethanol and water for injection, and comprising sucrose, 2-hydroxypropyl-β-cyclodextrin, a buffer, and a suspension of a pharmaceutical composition of claim 24.

53. (canceled)

54. (canceled)

55. A pharmaceutical composition comprising a solid lyophile of the pharmaceutical solution of claim 52.

56. A vector comprising a nucleic acid sequence encoding the dsRNA of claim 1.

57. A cell comprising the dsRNA of claim 1.

58. (canceled)

59. A method for preventing, treating, reducing, or ameliorating one or more symptoms of a Coronavirus infection in a mammal in need thereof, the method comprising administering to the mammal a therapeutically effective amount of the pharmaceutical composition of claim 24; and, optionally, selecting or identifying said mammal to receive a medication for the Coronavirus infection, which can be performed by clinical and/or diagnostic evaluation.

60. (canceled)

61. (canceled)

62. (canceled)

63. (canceled)


SEQUENCE LISTING (174 sequences)

SEQ ID NO: 1 - RNA, 19 nt, Artificial Sequence
  Synthetic oligonucleotide
ucgucaacaaccuagacaa

SEQ ID NO: 2 - RNA, 19 nt, Artificial Sequence
  Synthetic oligonucleotide
uugucuagguuguugacga

SEQ ID NO: 3 - RNA, 19 nt, Artificial Sequence
  Synthetic oligonucleotide
aagaauagagcucgcaccg

SEQ ID NO: 4 - RNA, 19 nt, Artificial Sequence
  Synthetic oligonucleotide
cggugcgagcucuauucuu

SEQ ID NO: 5 - RNA, 19 nt, Artificial Sequence
  Synthetic oligonucleotide
agaauagagcucgcaccgu

SEQ ID NO: 6 - RNA, 19 nt, Artificial Sequence
  Synthetic oligonucleotide
acggugcgagcucuauucu

SEQ ID NO: 7 - RNA, 19 nt, Artificial Sequence
  Synthetic oligonucleotide
agagccaugccuaacaugc

SEQ ID NO: 8 - RNA, 19 nt, Artificial Sequence
  Synthetic oligonucleotide
gcauguuaggcauggcucu

SEQ ID NO: 9 - RNA, 19 nt, Artificial Sequence
  Synthetic oligonucleotide
aucacccgcgaagaagcua

SEQ ID NO: 10 - RNA, 19 nt, Artificial Sequence
  Synthetic oligonucleotide
uagcuucuucgcgggugau

SEQ ID NO: 11 - RNA, 19 nt, Artificial Sequence
  Synthetic oligonucleotide
ucacccgcgaagaagcuau

SEQ ID NO: 12 - RNA, 19 nt, Artificial Sequence
  Synthetic oligonucleotide
auagcuucuucgcggguga

SEQ ID NO: 13 - RNA, 19 nt, Artificial Sequence
  Synthetic oligonucleotide
ugcucaccuauaacaaagu

SEQ ID NO: 14 - RNA, 19 nt, Artificial Sequence
  Synthetic oligonucleotide
acuuuguuauaggugagca

SEQ ID NO: 15 - RNA, 19 nt, Artificial Sequence
  Synthetic oligonucleotide
uagcuggugucucuaucug

SEQ ID NO: 16 - RNA, 19 nt, Artificial Sequence
  Synthetic oligonucleotide
cagauagagacaccagcua

SEQ ID NO: 17 - RNA, 19 nt, Artificial Sequence
  Synthetic oligonucleotide
ucucuaucuguaguacuau

SEQ ID NO: 18 - RNA, 19 nt, Artificial Sequence
  Synthetic oligonucleotide
auaguacuacagauagaga

SEQ ID NO: 19 - RNA, 19 nt, Artificial Sequence
  Synthetic oligonucleotide
cucuaucuguaguacuaug

SEQ ID NO: 20 - RNA, 19 nt, Artificial Sequence
  Synthetic oligonucleotide
cauaguacuacagauagag

SEQ ID NO: 21 - RNA, 19 nt, Artificial Sequence
  Synthetic oligonucleotide
gaugccacaacugcuuaug

SEQ ID NO: 22 - RNA, 19 nt, Artificial Sequence
  Synthetic oligonucleotide
cauaagcaguuguggcauc

SEQ ID NO: 23 - RNA, 19 nt, Artificial Sequence
  Synthetic oligonucleotide
gguacugguaagagucauu

SEQ ID NO: 24 - RNA, 19 nt, Artificial Sequence
  Synthetic oligonucleotide
aaugacucuuaccaguacc

SEQ ID NO: 25 - RNA, 19 nt, Artificial Sequence
  Synthetic oligonucleotide
auagguccagacauguucc

SEQ ID NO: 26 - RNA, 19 nt, Artificial Sequence
  Synthetic oligonucleotide
ggaacaugucuggaccuau

SEQ ID NO: 27 - RNA, 19 nt, Artificial Sequence
  Synthetic oligonucleotide
auaacagaugcgcaaacag

SEQ ID NO: 28 - RNA, 19 nt, Artificial Sequence
  Synthetic oligonucleotide
cuguuugcgcaucuguuau

SEQ ID NO: 29 - RNA, 19 nt, Artificial Sequence
  Synthetic oligonucleotide
uaacagaugcgcaaacagg

SEQ ID NO: 30 - RNA, 19 nt, Artificial Sequence
  Synthetic oligonucleotide
ccuguuugcgcaucuguua

SEQ ID NO: 31 - RNA, 19 nt, Artificial Sequence
  Synthetic oligonucleotide
agaugcgcaaacagguuca

SEQ ID NO: 32 - RNA, 19 nt, Artificial Sequence
  Synthetic oligonucleotide
ugaaccuguuugcgcaucu

SEQ ID NO: 33 - RNA, 19 nt, Artificial Sequence
  Synthetic oligonucleotide
acacuagccauccuuacug

SEQ ID NO: 34 - RNA, 19 nt, Artificial Sequence
  Synthetic oligonucleotide
caguaaggauggcuagugu

SEQ ID NO: 35 - RNA, 19 nt, Artificial Sequence
  Synthetic oligonucleotide
acuagccauccuuacugcg

SEQ ID NO: 36 - RNA, 19 nt, Artificial Sequence
  Synthetic oligonucleotide
cgcaguaaggauggcuagu

SEQ ID NO: 37 - RNA, 19 nt, Artificial Sequence
  Synthetic oligonucleotide
caauaauacugcgucuugg

SEQ ID NO: 38 - RNA, 19 nt, Artificial Sequence
  Synthetic oligonucleotide
ccaagacgcaguauuauug

SEQ ID NO: 39 - RNA, 19 nt, Artificial Sequence
  Synthetic oligonucleotide
aauagcaguccagaugacc

SEQ ID NO: 40 - RNA, 19 nt, Artificial Sequence
  Synthetic oligonucleotide
ggucaucuggacugcuauu

SEQ ID NO: 41 - RNA, 19 nt, Artificial Sequence
  Synthetic oligonucleotide
uucuacuaccuaggaacug

SEQ ID NO: 42 - RNA, 19 nt, Artificial Sequence
  Synthetic oligonucleotide
caguuccuagguaguagaa

SEQ ID NO: 43 - RNA, 19 nt, Artificial Sequence
  Synthetic oligonucleotide
ugccaaaaggcuucuacgc

SEQ ID NO: 44 - RNA, 19 nt, Artificial Sequence
  Synthetic oligonucleotide
gcguagaagccuuuuggca

SEQ ID NO: 45 - RNA, 19 nt, Artificial Sequence
  Synthetic oligonucleotide
cagauugaaccagcuugag

SEQ ID NO: 46 - RNA, 19 nt, Artificial Sequence
  Synthetic oligonucleotide
cucaagcugguucaaucug

SEQ ID NO: 47 - RNA, 19 nt, Artificial Sequence
  Synthetic oligonucleotide
agauugaaccagcuugaga

SEQ ID NO: 48 - RNA, 19 nt, Artificial Sequence
  Synthetic oligonucleotide
ucucaagcugguucaaucu

SEQ ID NO: 49 - RNA, 19 nt, Artificial Sequence
  Synthetic oligonucleotide
cugguaaaggccaacaaca

SEQ ID NO: 50 - RNA, 19 nt, Artificial Sequence
  Synthetic oligonucleotide
uguuguuggccuuuaccag

SEQ ID NO: 51 - RNA, 19 nt, Artificial Sequence
  Synthetic oligonucleotide
gguaaaggccaacaacaac

SEQ ID NO: 52 - RNA, 19 nt, Artificial Sequence
  Synthetic oligonucleotide
guuguuguuggccuuuacc

SEQ ID NO: 53 - RNA, 19 nt, Artificial Sequence
  Synthetic oligonucleotide
ggccaaacugucacuaaga

SEQ ID NO: 54 - RNA, 19 nt, Artificial Sequence
  Synthetic oligonucleotide
ucuuagugacaguuuggcc

SEQ ID NO: 55 - RNA, 19 nt, Artificial Sequence
  Synthetic oligonucleotide
gccaaacugucacuaagaa

SEQ ID NO: 56 - RNA, 19 nt, Artificial Sequence
  Synthetic oligonucleotide
uucuuagugacaguuuggc

SEQ ID NO: 57 - RNA, 19 nt, Artificial Sequence
  Synthetic oligonucleotide
uuaaucucacauagcaauc

SEQ ID NO: 58 - RNA, 19 nt, Artificial Sequence
  Synthetic oligonucleotide
gauugcuaugugagauuaa

SEQ ID NO: 59 - RNA, 19 nt, Artificial Sequence
  Synthetic oligonucleotide
gacuugaaagagccaccac

SEQ ID NO: 60 - RNA, 19 nt, Artificial Sequence
  Synthetic oligonucleotide
gugguggcucuuucaaguc

SEQ ID NO: 61 - RNA, 21 nt, Artificial Sequence
  Synthetic oligonucleotide
  modified_base (20)...(21): 2′-OMe-nucleotide
ucgucaacaaccuagacaauu

SEQ ID NO: 62 - RNA, 21 nt, Artificial Sequence
  Synthetic oligonucleotide
  modified_base (20)...(21): 2′-OMe-nucleotide
uugucuagguuguugacgauu

SEQ ID NO: 63 - RNA, 21 nt, Artificial Sequence
  Synthetic oligonucleotide
  modified_base (20)...(21): 2′-OMe-nucleotide
aagaauagagcucgcaccguu

SEQ ID NO: 64 - RNA, 21 nt, Artificial Sequence
  Synthetic oligonucleotide
  modified_base (20)...(21): 2′-OMe-nucleotide
cggugcgagcucuauucuuuu

SEQ ID NO: 65 - RNA, 21 nt, Artificial Sequence
  Synthetic oligonucleotide
  modified_base (20)...(21): 2′-OMe-nucleotide
agaauagagcucgcaccguuu

SEQ ID NO: 66 - RNA, 21 nt, Artificial Sequence
  Synthetic oligonucleotide
  modified_base (20)...(21): 2′-OMe-nucleotide
acggugcgagcucuauucuuu

SEQ ID NO: 67 - RNA, 21 nt, Artificial Sequence
  Synthetic oligonucleotide
  modified_base (20)...(21): 2′-OMe-nucleotide
agagccaugccuaacaugcuu

SEQ ID NO: 68 - RNA, 21 nt, Artificial Sequence
  Synthetic oligonucleotide
  modified_base (20)...(21): 2′-OMe-nucleotide
gcauguuaggcauggcucuuu

SEQ ID NO: 69 - RNA, 21 nt, Artificial Sequence
  Synthetic oligonucleotide
  modified_base (20)...(21): 2′-OMe-nucleotide
aucacccgcgaagaagcuauu

SEQ ID NO: 70 - RNA, 21 nt, Artificial Sequence
  Synthetic oligonucleotide
  modified_base (20)...(21): 2′-OMe-nucleotide
uagcuucuucgcgggugauuu

SEQ ID NO: 71 - RNA, 21 nt, Artificial Sequence
  Synthetic oligonucleotide
  modified_base (20)...(21): 2′-OMe-nucleotide
ucacccgcgaagaagcuauuu

SEQ ID NO: 72 - RNA, 21 nt, Artificial Sequence
  Synthetic oligonucleotide
  modified_base (20)...(21): 2′-OMe-nucleotide
auagcuucuucgcgggugauu

SEQ ID NO: 73 - RNA, 21 nt, Artificial Sequence
  Synthetic oligonucleotide
  modified_base (20)...(21): 2′-OMe-nucleotide
ugcucaccuauaacaaaguuu

SEQ ID NO: 74 - RNA, 21 nt, Artificial Sequence
  Synthetic oligonucleotide
  modified_base (20)...(21): 2′-OMe-nucleotide
acuuuguuauaggugagcauu

SEQ ID NO: 75 - RNA, 21 nt, Artificial Sequence
  Synthetic oligonucleotide
  modified_base (20)...(21): 2′-OMe-nucleotide
uagcuggugucucuaucuguu

SEQ ID NO: 76 - RNA, 21 nt, Artificial Sequence
  Synthetic oligonucleotide
  modified_base (20)...(21): 2′-OMe-nucleotide
cagauagagacaccagcuauu

SEQ ID NO: 77 - RNA, 21 nt, Artificial Sequence
  Synthetic oligonucleotide
  modified_base (20)...(21): 2′-OMe-nucleotide
ucucuaucuguaguacuauuu

SEQ ID NO: 78 - RNA, 21 nt, Artificial Sequence
  Synthetic oligonucleotide
  modified_base (20)...(21): 2′-OMe-nucleotide
auaguacuacagauagagauu

SEQ ID NO: 79 - RNA, 21 nt, Artificial Sequence
  Synthetic oligonucleotide
  modified_base (20)...(21): 2′-OMe-nucleotide
cucuaucuguaguacuauguu

SEQ ID NO: 80 - RNA, 21 nt, Artificial Sequence
  Synthetic oligonucleotide
  modified_base (20)...(21): 2′-OMe-nucleotide
cauaguacuacagauagaguu

SEQ ID NO: 81 - RNA, 21 nt, Artificial Sequence
  Synthetic oligonucleotide
  modified_base (20)...(21): 2′-OMe-nucleotide
gaugccacaacugcuuauguu

SEQ ID NO: 82 - RNA, 21 nt, Artificial Sequence
  Synthetic oligonucleotide
  modified_base (20)...(21): 2′-OMe-nucleotide
cauaagcaguuguggcaucuu

SEQ ID NO: 83 - RNA, 21 nt, Artificial Sequence
  Synthetic oligonucleotide
  modified_base (20)...(21): 2′-OMe-nucleotide
gguacugguaagagucauuuu

SEQ ID NO: 84 - RNA, 21 nt, Artificial Sequence
  Synthetic oligonucleotide
  modified_base (20)...(21): 2′-OMe-nucleotide
aaugacucuuaccaguaccuu

SEQ ID NO: 85 - RNA, 21 nt, Artificial Sequence
  Synthetic oligonucleotide
  modified_base (20)...(21): 2′-OMe-nucleotide
auagguccagacauguuccuu

SEQ ID NO: 86 - RNA, 21 nt, Artificial Sequence
  Synthetic oligonucleotide
  modified_base (20)...(21): 2′-OMe-nucleotide
ggaacaugucuggaccuauuu

SEQ ID NO: 87 - RNA, 21 nt, Artificial Sequence
  Synthetic oligonucleotide
  modified_base (20)...(21): 2′-OMe-nucleotide
auaacagaugcgcaaacaguu

SEQ ID NO: 88 - RNA, 21 nt, Artificial Sequence
  Synthetic oligonucleotide
  modified_base (20)...(21): 2′-OMe-nucleotide
cuguuugcgcaucuguuauuu

SEQ ID NO: 89 - RNA, 21 nt, Artificial Sequence
  Synthetic oligonucleotide
  modified_base (20)...(21): 2′-OMe-nucleotide
uaacagaugcgcaaacagguu

SEQ ID NO: 90 - RNA, 21 nt, Artificial Sequence
  Synthetic oligonucleotide
  modified_base (20)...(21): 2′-OMe-nucleotide
ccuguuugcgcaucuguuauu

SEQ ID NO: 91 - RNA, 21 nt, Artificial Sequence
  Synthetic oligonucleotide
  modified_base (20)...(21): 2′-OMe-nucleotide
agaugcgcaaacagguucauu

SEQ ID NO: 92 - RNA, 21 nt, Artificial Sequence
  Synthetic oligonucleotide
  modified_base (20)...(21): 2′-OMe-nucleotide
ugaaccuguuugcgcaucuuu

SEQ ID NO: 93 - RNA, 21 nt, Artificial Sequence
  Synthetic oligonucleotide
  modified_base (20)...(21): 2′-OMe-nucleotide
acacuagccauccuuacuguu

SEQ ID NO: 94 - RNA, 21 nt, Artificial Sequence
  Synthetic oligonucleotide
  modified_base (20)...(21): 2′-OMe-nucleotide
caguaaggauggcuaguguuu

SEQ ID NO: 95 - RNA, 21 nt, Artificial Sequence
  Synthetic oligonucleotide
  modified_base (20)...(21): 2′-OMe-nucleotide
acuagccauccuuacugcguu

SEQ ID NO: 96 - RNA, 21 nt, Artificial Sequence
  Synthetic oligonucleotide
  modified_base (20)...(21): 2′-OMe-nucleotide
cgcaguaaggauggcuaguuu

SEQ ID NO: 97 - RNA, 21 nt, Artificial Sequence
  Synthetic oligonucleotide
  modified_base (20)...(21): 2′-OMe-nucleotide
caauaauacugcgucuugguu

SEQ ID NO: 98 - RNA, 21 nt, Artificial Sequence
  Synthetic oligonucleotide
  modified_base (20)...(21): 2′-OMe-nucleotide
ccaagacgcaguauuauuguu

SEQ ID NO: 99 - RNA, 21 nt, Artificial Sequence
  Synthetic oligonucleotide
  modified_base (20)...(21): 2′-OMe-nucleotide
aauagcaguccagaugaccuu

SEQ ID NO: 100 - RNA, 21 nt, Artificial Sequence
  Synthetic oligonucleotide
  modified_base (20)...(21): 2′-OMe-nucleotide
ggucaucuggacugcuauuuu

SEQ ID NO: 101 - RNA, 21 nt, Artificial Sequence
  Synthetic oligonucleotide
  modified_base (20)...(21): 2′-OMe-nucleotide
uucuacuaccuaggaacuguu

SEQ ID NO: 102 - RNA, 21 nt, Artificial Sequence
  Synthetic oligonucleotide
  modified_base (20)...(21): 2′-OMe-nucleotide
caguuccuagguaguagaauu

SEQ ID NO: 103 - RNA, 21 nt, Artificial Sequence
  Synthetic oligonucleotide
  modified_base (20)...(21): 2′-OMe-nucleotide
ugccaaaaggcuucuacgcuu

SEQ ID NO: 104 - RNA, 21 nt, Artificial Sequence
  Synthetic oligonucleotide
  modified_base (20)...(21): 2′-OMe-nucleotide
gcguagaagccuuuuggcauu

SEQ ID NO: 105 - RNA, 21 nt, Artificial Sequence
  Synthetic oligonucleotide
  modified_base (20)...(21): 2′-OMe-nucleotide
cagauugaaccagcuugaguu

SEQ ID NO: 106 - RNA, 21 nt, Artificial Sequence
  Synthetic oligonucleotide
  modified_base (20)...(21): 2′-OMe-nucleotide
cucaagcugguucaaucuguu

SEQ ID NO: 107 - RNA, 21 nt, Artificial Sequence
  Synthetic oligonucleotide
  modified_base (20)...(21): 2′-OMe-nucleotide
agauugaaccagcuugagauu

SEQ ID NO: 108 - RNA, 21 nt, Artificial Sequence
  Synthetic oligonucleotide
  modified_base (20)...(21): 2′-OMe-nucleotide
ucucaagcugguucaaucuuu

SEQ ID NO: 109 - RNA, 21 nt, Artificial Sequence
  Synthetic oligonucleotide
  modified_base (20)...(21): 2′-OMe-nucleotide
cugguaaaggccaacaacauu

SEQ ID NO: 110 - RNA, 21 nt, Artificial Sequence
  Synthetic oligonucleotide
  modified_base (20)...(21): 2′-OMe-nucleotide
uguuguuggccuuuaccaguu

SEQ ID NO: 111 - RNA, 21 nt, Artificial Sequence
  Synthetic oligonucleotide
  modified_base (20)...(21): 2′-OMe-nucleotide
gguaaaggccaacaacaacuu

SEQ ID NO: 112 - RNA, 21 nt, Artificial Sequence
  Synthetic oligonucleotide
  modified_base (20)...(210): 2′-OMe-nucleotide
guuguuguuggccuuuaccuu

SEQ ID NO: 113 - RNA, 21 nt, Artificial Sequence
  Synthetic oligonucleotide
  modified_base (20)...(21): 2′-OMe-nucleotide
ggccaaacugucacuaagauu

SEQ ID NO: 114 - RNA, 21 nt, Artificial Sequence
  Synthetic oligonucleotide
  modified_base (20)...(21): 2′-OMe-nucleotide
ucuuagugacaguuuggccuu

SEQ ID NO: 115 - RNA, 21 nt, Artificial Sequence
  Synthetic oligonucleotide
  modified_base (20)...(21): 2′-OMe-nucleotide
gccaaacugucacuaagaauu

SEQ ID NO: 116 - RNA, 21 nt, Artificial Sequence
  Synthetic oligonucleotide
  modified_base (20)...(21): 2′-OMe-nucleotide
uucuuagugacaguuuggcuu

SEQ ID NO: 117 - RNA, 21 nt, Artificial Sequence
  Synthetic oligonucleotide
  modified_base (20)...(21): 2′-OMe-nucleotide
uuaaucucacauagcaaucuu

SEQ ID NO: 118 - RNA, 21 nt, Artificial Sequence
  Synthetic oligonucleotide
  modified_base (20)...(21): 2′-OMe-nucleotide
gauugcuaugugagauuaauu

SEQ ID NO: 119 - RNA, 21 nt, Artificial Sequence
  Synthetic oligonucleotide
  modified_base (20)...(21): 2′-OMe-nucleotide
gacuugaaagagccaccacuu

SEQ ID NO: 120 - RNA, 21 nt, Artificial Sequence
  Synthetic oligonucleotide
  modified_base (20)...(21): 2′-OMe-nucleotide
gugguggcucuuucaagucuu

SEQ ID NO: 121 - RNA, 21 nt, Artificial Sequence
  Synthetic oligonucleotide
  modified_base 13: 2′-OMe-nucleotide
  modified_base 17: 2′-OMe-nucleotide
  modified_base (20)...(21): 2′-OMe-nucleotide
ucgucaacaaccuagacaauu

SEQ ID NO: 122 - RNA, 21 nt, Artificial Sequence
  Synthetic oligonucleotide
  modified_base 6: 2′-OMe-nucleotide
  modified_base (20)...(21): 2′-OMe-nucleotide
uugucuagguuguugacgauu

SEQ ID NO: 123 - RNA, 21 nt, Artificial Sequence
  Synthetic oligonucleotide
  modified_base 2: 2′-OMe-nucleotide
  modified_base (12)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 124 - RNA, 21 nt, Artificial Sequence
  Synthetic oligonucleotide
  modified_base (1)...(2): 2′-OMe-nucleotide
  modified_base 4: 2′-OMe-nucleotide
  modified_base 6: 2′-OMe-nucleotide
  modified_base (20)...(21): 2′-OMe-nucleotide
uugucuagguuguugacgauu

SEQ ID NO: 125 - RNA, 21 nt, Artificial Sequence
  Synthetic oligonucleotide
  modified_base 5: 2′-OMe-nucleotide
  modified_base 8: 2′-OMe-nucleotide
  modified_base 13: 2′-OMe-nucleotide
  modified_base 17: 2′-OMe-nucleotide
  mRNA (20)...(21): 2′-OMe-nucleotide
ucgucaacaaccuagacaauu

SEQ ID NO: 126 - RNA, 21 nt, Artificial Sequence
  Synthetic oligonucleotide
  modified_base 6: 2′-OMe-nucleotide
  modified_base (20)...(21): 2′-OMe-nucleotide
uugucuagguuguugacgauu

SEQ ID NO: 127 - RNA, 21 nt, Artificial Sequence
  Synthetic oligonucleotide
  modified_base 5: 2′-OMe-nucleotide
  modified_base 8: 2′-OMe-nucleotide
  modified_base 13: 2′-OMe-nucleotide
  modified_base 17: 2′-OMe-nucleotide
  modified_base (20)...(21): 2′-OMe-nucleotide
ucgucaacaaccuagacaauu

SEQ ID NO: 128 - RNA, 21 nt, Artificial Sequence
  Synthetic oligonucleotide
  modified_base (1)...(2): 2′-OMe-nucleotide
  modified_base 4: 2′-OMe-nucleotide
  modified_base 6: 2′-OMe-nucleotide
  modified_base (20)...(21): 2′-OMe-nucleotide
uugucuagguuguugacgauu

SEQ ID NO: 129 - RNA, 21 nt, Artificial Sequence
  Synthetic oligonucleotide
  modified_base (1)...(2): 2′-OMe-nucleotide
  modified_base (4)...(5): 2′-OMe-nucleotide
  modified_base 8: 2′-OMe-nucleotide
  modified_base (11)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 130 - RNA, 21 nt, Artificial Sequence
  Synthetic oligonucleotide
  modified_base 6: 2′-OMe-nucleotide
  modified_base (20)...(21): 2′-OMe-nucleotide
uugucuagguuguugacgauu

SEQ ID NO: 131 - RNA, 21 nt, Artificial Sequence
  Synthetic oligonucleotide
  modified_base (1)...(2): 2′-OMe-nucleotide
  modified_base (4)...(5): 2′-OMe-nucleotide
  modified_base 8: 2′-OMe-nucleotide
  modified_base (11)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 132 - RNA, 21 nt, Artificial Sequence
  Synthetic oligonucleotide
  modified_base (1)...(2): 2′-OMe-nucleotide
  modified_base 4: 2′-OMe-nucleotide
  modified_base 6: 2′-OMe-nucleotide
  modified_base (20)...(21): 2′-OMe-nucleotide
uugucuagguuguugacgauu

SEQ ID NO: 133 - RNA, 21 nt, Artificial Sequence
  Synthetic oligonucleotide
  modified_base 5: 2′-OMe-nucleotide
  modified_base 8: 2′-OMe-nucleotide
  modified_base 13: 2′-OMe-nucleotide
  modified_base 17: 2′-OMe-nucleotide
  modified_base (20)...(21): 2′-OMe-nucleotide
ucgucaacaaccuagacaauu

SEQ ID NO: 134 - RNA, 22 nt, Artificial Sequence
  Synthetic oligonucleotide
  modified_base 3: 2′-deoxy-nucleotide
  modified_base 5: 2′-deoxy-nucleotide
  modified_base 6: 2′-OMe-nucleotide
  modified_base 7: 2′-deoxy-nucleotide
  modified_base (20)...(21): 2′-OMe-nucleotide
uugucuagguuguugacgauuu

SEQ ID NO: 135 - RNA, 21 nt, Artificial Sequence
  Synthetic oligonucleotide
  modified_base 5: 2′-OMe-nucleotide
  modified_base 8: 2′-OMe-nucleotide
  modified_base 13: 2′-OMe-nucleotide
  modified_base 17: 2′-OMe-nucleotide
  modified_base (20)...(21): 2′-OMe-nucleotide
ucgucaacaaccuagacaauu

SEQ ID NO: 136 - RNA, 21 nt, Artificial Sequence
  Synthetic oligonucleotide
  modified_base 4: 2′-deoxy-nucleotide
  modified_base 6: 2′-OMe-nucleotide
  modified_base 8: 2′-deoxy-nucleotide
  modified_base (20)...(21): 2′-OMe-nucleotide
uugucuagguuguugacgauu

SEQ ID NO: 137 - RNA, 21 nt, Artificial Sequence
  Synthetic oligonucleotide
  modified_base 5: 2′-OMe-nucleotide
  modified_base 8: 2′-OMe-nucleotide
  modified_base 13: 2′-OMe-nucleotide
  modified_base 17: 2′-OMe-nucleotide
  modified_base (20)...(21): 2′-OMe-nucleotide
ucgucaacaaccuagacaauu

SEQ ID NO: 138 - RNA, 21 nt, Artificial Sequence
  Synthetic oligonucleotide
  modified_base (1)...(2): 2′-OMe-nucleotide
  modified_base 3: 2′-deoxy-nucleotide
  modified_base 4: 2′-OMe-nucleotide
  modified_base 5: 2′-deoxy-nucleotide
  modified_base 6: 2′-OMe-nucleotide
  modified_base (7)...(7): 2′-deoxy-nucleotide
  modified_base (20)...(21): 2′-OMe-nucleotide
uugucuagguuguugacgauu

SEQ ID NO: 139 - RNA, 21 nt, Artificial Sequence
  Synthetic oligonucleotide
  modified_base 2: 2′-OMe-nucleotide
  modified_base (12)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 140 - RNA, 21 nt, Artificial Sequence
  Synthetic oligonucleotide
  modified_base 3: 2′-deoxy-nucleotide
  modified_base 5: 2′-deoxy-nucleotide
  modified_base 6: 2′-OMe-nucleotide
  modified_base 7: 2′-deoxy-nucleotide
  modified_base (20)...(21): 2′-OMe-nucleotide
uugucuagguuguugacgauu

SEQ ID NO: 141 - RNA, 21 nt, Artificial Sequence
  Synthetic oligonucleotide
  modified_base 2: 2′-OMe-nucleotide
  modified_base (12)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 142 - RNA, 21 nt, Artificial Sequence
  Synthetic oligonucleotide
  modified_base 4: 2′-deoxy-nucleotide
  modified_base 6: 2′-OMe-nucleotide
  modified_base 8: 2′-deoxy-nucleotide
  modified_base (20)...(21): 2′-OMe-nucleotide
uugucuagguuguugacgauu

SEQ ID NO: 143 - RNA, 21 nt, Artificial Sequence
  Synthetic oligonucleotide
  modified_base 2: 2′-OMe-nucleotide
  modified_base (12)...(13): 2′-OMe-nucleotide
  modified_base 17: 2′-OMe-nucleotide
  modified_base (20)...(21): 2′-OMe-nucleotide
ucgucaacaaccuagacaauu

SEQ ID NO: 144 - RNA, 21 nt, Artificial Sequence
  Synthetic oligonucleotide
  modified_base (1)...(2): 2′-OMe-nucleotide
  modified_base 3: 2′-deoxy-nucleotide
  modified_base 4: 2′-OMe-nucleotide
  modified_base 5: 2′-deoxy-nucleotide
  modified_base 6: 2′-OMe-nucleotide
  modified_base (7)...(7): 2′-deoxy-nucleotide
  modified_base (20)...(21): 2′-OMe-nucleotide
uugucuagguuguugacgauu

SEQ ID NO: 145 - RNA, 21 nt, Artificial Sequence
  Synthetic oligonucleotide
  modified_base 17: 2′-OMe-nucleotide
  modified_base (20)...(21): 2′-OMe-nucleotide
ucacccgcgaagaagcuauuu

SEQ ID NO: 146 - RNA, 21 nt, Artificial Sequence
  Synthetic oligonucleotide
  modified_base 2: 2′-OMe-nucleotide
  modified_base (20)...(21): 2′-OMe-nucleotide
auagcuucuucgcgggugauu

SEQ ID NO: 147 - RNA, 21 nt, Artificial Sequence
  Synthetic oligonucleotide
  modified_base (14)...(17): 2′-OMe-nucleotide
  modified_base (20)...(21): 2′-OMe-nucleotide
ucacccgcgaagaagcuauuu

SEQ ID NO: 148 - RNA, 21 nt, Artificial Sequence
  Synthetic oligonucleotide
  modified_base 2: 2′-OMe-nucleotide
  modified_base 10: 2′-OMe-nucleotide
  modified_base 15: 2′-OMe-nucleotide
  modified_base (20)...(21): 2′-OMe-nucleotide
auagcuucuucgcgggugauu

SEQ ID NO: 149 - RNA, 21 nt, Artificial Sequence
  Synthetic oligonucleotide
  modified_base 2: 2′-OMe-nucleotide
  modified_base 17: 2′-OMe-nucleotide
  modified_base (20)...(21): 2′-OMe-nucleotide
ucacccgcgaagaagcuauuu

SEQ ID NO: 150 - RNA, 21 nt, Artificial Sequence
  Synthetic oligonucleotide
  modified_base 2: 2′-OMe-nucleotide
  modified_base (20)...(21): 2′-OMe-nucleotide
auagcuucuucgcgggugauu

SEQ ID NO: 151 - RNA, 21 nt, Artificial Sequence
  Synthetic oligonucleotide
  modified_base 2: 2′-OMe-nucleotide
  modified_base 17: 2′-OMe-nucleotide
  modified_base (20)...(21): 2′-OMe-nucleotide
ucacccgcgaagaagcuauuu

SEQ ID NO: 152 - RNA, 21 nt, Artificial Sequence
  Synthetic oligonucleotide
  modified_base 2: 2′-OMe-nucleotide
  modified_base 10: 2′-OMe-nucleotide
  modified_base 15: 2′-OMe-nucleotide
  modified_base (20)...(21): 2′-OMe-nucleotide
auagcuucuucgcgggugauu

SEQ ID NO: 153 - RNA, 21 nt, Artificial Sequence
  Synthetic oligonucleotide
  modified_base (1)...(2): 2′-OMe-nucleotide
  modified_base (4)...(6): 2′-OMe-nucleotide
  modified_base 8: 2′-OMe-nucleotide
  modified_base (16)...(17): 2′-OMe-nucleotide
  modified_base (19)...(21): 2′-OMe-nucleotide
ucacccgcgaagaagcuauuu

SEQ ID NO: 154 - RNA, 21 nt, Artificial Sequence
  Synthetic oligonucleotide
  modified_base 2: 2′-OMe-nucleotide
  modified_base (20)...(21): 2′-OMe-nucleotide
auagcuucuucgcgggugauu

SEQ ID NO: 155 - RNA, 21 nt, Artificial Sequence
  Synthetic oligonucleotide
  modified_base (1)...(2): 2′-OMe-nucleotide
  modified_base (4)...(6): 2′-OMe-nucleotide
  modified_base 8: 2′-OMe-nucleotide
  modified_base (16)...(17): 2′-OMe-nucleotide
  modified_base (19)...(21): 2′-OMe-nucleotide
ucacccgcgaagaagcuauuu

SEQ ID NO: 156 - RNA, 21 nt, Artificial Sequence
  Synthetic oligonucleotide
  modified_base 2: 2′-OMe-nucleotide
  modified_base 10: 2′-OMe-nucleotide
  modified_base 15: 2′-OMe-nucleotide
  modified_base (20)...(21): 2′-OMe-nucleotide
auagcuucuucgcgggugauu

SEQ ID NO: 157 - RNA, 21 nt, Artificial Sequence
  Synthetic oligonucleotide
  modified_base 2: 2′-OMe-nucleotide
  modified_base 17: 2′-OMe-nucleotide
  modified_base (20)...(21): 2′-OMe-nucleotide
ucacccgcgaagaagcuauuu

SEQ ID NO: 158 - RNA, 21 nt, Artificial Sequence
  Synthetic oligonucleotide
  modified_base 2: 2′-OMe-nucleotide
  modified_base 3: 2′-deoxy-nucleotide
  modified_base 5: 2′-deoxy-nucleotide
  modified_base 7: 2′-deoxy-nucleotide
  modified_base (20)...(21): 2′-OMe-nucleotide
auagcuucuucgcgggugauu

SEQ ID NO: 159 - RNA, 21 nt, Artificial Sequence
  Synthetic oligonucleotide
  modified_base 2: 2′-OMe-nucleotide
  modified_base 17: 2′-OMe-nucleotide
  modified_base (20)...(21): 2′-OMe-nucleotide
ucacccgcgaagaagcuauuu

SEQ ID NO: 160 - RNA, 21 nt, Artificial Sequence
  Synthetic oligonucleotide
  modified_base 2: 2′-OMe-nucleotide
  modified_base 4: 2′-deoxy-nucleotide
  modified_base 6: 2′-deoxy-nucleotide
  modified_base 8: 2′-deoxy-nucleotide
  modified_base (20)...(21): 2′-OMe-nucleotide
auagcuucuucgcgggugauu

SEQ ID NO: 161 - RNA, 21 nt, Artificial Sequence
  Synthetic oligonucleotide
  modified_base 2: 2′-OMe-nucleotide
  modified_base 17: 2′-OMe-nucleotide
  modified_base (20)...(21): 2′-OMe-nucleotide
ucacccgcgaagaagcuauuu

SEQ ID NO: 162 - RNA, 21 nt, Artificial Sequence
  Synthetic oligonucleotide
  modified_base 2: 2′-OMe-nucleotide
  modified_base 3: 2′-deoxy-nucleotide
  modified_base 5: 2′-deoxy-nucleotide
  modified_base 7: 2′-deoxy-nucleotide
  modified_base 10: 2′-OMe-nucleotide
  modified_base (15)...(15): 2′-OMe-nucleotide
  modified_base (20)...(21): 2′-OMe-nucleotide
auagcuucuucgcgggugauu

SEQ ID NO: 163 - RNA, 21 nt, Artificial Sequence
  Synthetic oligonucleotide
  modified_base (14)...(17): 2′-OMe-nucleotide
  modified_base (20)...(21): 2′-OMe-nucleotide
ucacccgcgaagaagcuauuu

SEQ ID NO: 164 - RNA, 21 nt, Artificial Sequence
  Synthetic oligonucleotide
  modified_base 2: 2′-OMe-nucleotide
  modified_base 3: 2′-deoxy-nucleotide
  modified_base 5: 2′-deoxy-nucleotide
  modified_base 7: 2′-deoxy-nucleotide
  modified_base (20)...(21): 2′-OMe-nucleotide
auagcuucuucgcgggugauu

SEQ ID NO: 165 - RNA, 21 nt, Artificial Sequence
  Synthetic oligonucleotide
  modified_base (14)...(17): 2′-OMe-nucleotide
  modified_base (20)...(21): 2′-OMe-nucleotide
ucacccgcgaagaagcuauuu

SEQ ID NO: 166 - RNA, 21 nt, Artificial Sequence
  Synthetic oligonucleotide
  modified_base 2: 2′-OMe-nucleotide
  modified_base 4: 2′-deoxy-nucleotide
  modified_base 6: 2′-deoxy-nucleotide
  modified_base 8: 2′-deoxy-nucleotide
  modified_base (20)...(21): 2′-OMe-nucleotide
auagcuucuucgcgggugauu

SEQ ID NO: 167 - RNA, 21 nt, Artificial Sequence
  Synthetic oligonucleotide
  modified_base (14)...(17): 2′-OMe-nucleotide
  modified_base (20)...(21): 2′-OMe-nucleotide
ucacccgcgaagaagcuauuu

SEQ ID NO: 168 - RNA, 21 nt, Artificial Sequence
  Synthetic oligonucleotide
  modified_base 2: 2′-OMe-nucleotide
  modified_base 3: 2′-deoxy-nucleotide
  modified_base 5: 2′-deoxy-nucleotide
  modified_base 7: 2′-deoxy-nucleotide
  modified_base 10: 2′-OMe-nucleotide
  modified_base (15)...(15): 2′-OMe-nucleotide
  modified_base (20)...(21): 2′-OMe-nucleotide
auagcuucuucgcgggugauu

SEQ ID NO: 169 - DNA, 29903 bp, SARS-CoV-2 virus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 170 - DNA, 805 bp, Artificial Sequence
  Synthetic Construct
ctcgagaataactatatgctcacctataacaaagttgaaaacatgacaaccaagtcatcgtcaacaacctagacaaatcagctggtttcattagtgcaaagaatagagctcgcaccgtagctggtgtctctatctgtagtactatgaccaatagacagtaaatgtgatagagccatgcctaacatgcttagaattatggctcatcaggagatgccacaactgcttatgctaatagtgtttgggaccacctggtactggtaagagtcattttgctattggcctatgaaaactataggtccagacatgttcctcggaacttgtctaacatgtttatcacccgcgaagaagctataagacatgtacgaaactatttcataacagatgcgcaaacaggttcatctaagtgtgtgcttgctagttacactagccatccttactgcgcttcgattgtgtaaggtttacccaataatactgcgtcttggttcaccgctctcaattaacaccaatagcagtccagatgaccaaattggctactaagatggtatttctactacctaggaactgggccagaagctgggaacaacattgccaaaaggcttctacgcagaagggagcagtgctgcttgacagattgaaccagcttgagagcaaaatgtctggtaaaggccaacaacaacaaggccaaactgtcactaagaaatctgctgctgagtagttaactttaatctcacatagcaatctttaatcagtgtgtaacattagggaggacttgaaagagccaccacattttcaccgaggcggccgc

SEQ ID NO: 171 - DNA, 20 bp, Artificial Sequence
  Synthetic Construct
gtgttcgacaatggcagcat

SEQ ID NO: 172 - DNA, 19 bp, Artificial Sequence
  Synthetic Construct
gacaccctccaggaagcga

SEQ ID NO: 173 - DNA, 30 bp, Artificial Sequence
  Synthetic Construct
cctactaaattaaatgatctctgctttact

SEQ ID NO: 174 - DNA, 21 bp, Artificial Sequence
  Synthetic Construct
caagctataacgcagcctgta